(19) United States
(12) Patent Application Publication
(10) Pub. No.: US 2025/0122570 A1
(43) Pub. Date: Apr. 17, 2025

(54) EPIGENOME BIOMARKERS FOR IDENTIFYING ALZHEIMER'S DISEASE

(71) Applicant: Salk Institute for Biological Studies, La Jolla, CA (US)

(72) Inventors: Joseph R. Ecker, Carlsbad, CA (US); Bang-An Wang, La Jolla, CA (US); Wei Tian, San Diego, CA (US); Jeffrey R. Jones, La Jolla, CA (US); Fred H. Gage, La Jolla, CA (US)

(73) Assignee: Salk Institute for Biological Studies, La Jolla, CA (US)

(21) Appl. No.: 18/913,252

(22) Filed: Oct. 11, 2024

Related U.S. Application Data
(60) Provisional application No. 63/543,905, filed on Oct. 12, 2023.

(51) Int. Cl. C12Q 1/6883; C12N 5/077; C12N 5/0793; G01N 33/50

(52) U.S. Cl. CPC C12Q 1/6883; C12N 5/0619; C12N 5/0656; G01N 33/5058; C12Q 2600/136; C12Q 2600/154

(57) ABSTRACT

Methods are provided for identifying Alzheimer's disease cells or subjects, based on the methylation status of multiple methylation markers in genomic DNA. Also provided are methods for identifying therapeutic agents for treating Alzheimer's disease by monitoring changes in the methylation status of multiple methylation markers.

### CROSS REFERENCE TO RELATED APPLICATIONS

This application claims the benefit of U.S. Provisional Application No. 63/543,905, filed Oct. 12, 2023, which is herein incorporated by reference in its entirety.

### FIELD

Methods are provided for identifying Alzheimer's disease (AD) cells or subjects, based on the methylation status of multiple methylation markers in genomic DNA. Also provided are methods for identifying therapeutic agents for treating AD by monitoring changes in the methylation status of multiple methylation markers.

### BACKGROUND

The brain is the most complex organ in the human body containing billions of neuronal and non-neuronal cells with extensive diversity in gene expression, anatomy and functions. High throughput epigenomic sequencing is a powerful tool to elucidate the gene regulatory programs underlying such cellular complexity, which is critical for understanding normal and dysfunctional brain states. Considered the fifth base of DNA, methylated cytosines (5mCs) are the most common modified bases in mammalian genomes, providing an important epigenetic mechanism for the regulation of gene expression. Most 5mCs in vertebrate genomes occur at cytosine-guanine dinucleotides (CpGs). In vertebrate neuronal systems, however, 5mCs are also abundantly detected in non-CG (or CH, H=A, C, or T) contexts. Both CG- and CH-methylation (mCG and mCH, respectively) are highly dynamic during brain development and show remarkable cell type specificity. mCG and mCH are both essential for gene regulation and brain functions. In addition to DNA methylation, the expression of genes also requires proper spatial organization of the chromatin (3D chromatin conformation), usually represented as chromosome compartments, chromatin domains and DNA loops. Such spatial organization facilitates the interaction between gene promoters and their regulatory elements, providing additional critical layers of regulatory mechanisms. DNA methylation and chromatin conformation interplay and coordinate in regulating gene expression and these processes are highly correlated.

Studying human age-dependent disorders is a long-standing challenge, especially for inaccessible tissues like the human brain. Sporadic late-onset Alzheimer's disease (LOAD) accounts for 95% of all AD cases. Unlike the early-onset familial AD that is linked to genetic mutations in specific genes, such as those found in APP, PSEN1 and PSEN2 genes, LOAD is thought to be caused by a complex combination of multiple genes and environmental factors, largely aligning to several age-related co-morbidities. Elucidating the complex genetic background interactions and epigenetic regulation that likely contribute to LOAD is critical to developing targeted therapies. DNA methylation, the most studied epigenetic system in mammals, has been confirmed to play a crucial role in multiple human diseases such as cancer, imprinting and repeat-instability disorders. Intriguingly, aberrant DNA methylation is observed in normal aging processes, highlighting the link between proper epigenetic regulation and age-dependent cellular functions.

### SUMMARY

Provided herein are methods of identifying a subject as having or at risk of developing Alzheimer's disease (AD), such as late-onset AD (LOAD). In some aspects, the method includes obtaining sequence reads of a methylation sequencing assay covering genomic segments of a biological sample from the subject, wherein the genomic segments contain one or more of the genomic positions listed in Table 1 and/or Table 2; and identifying the subject as having or at risk of developing AD if at least one of the genomic positions has a different methylation status compared to a normal control, or identifying the subject as not having or at risk of developing AD if none of the genomic positions has a different methylation status compared to a normal control. In some aspects, the method further includes administering a therapeutically effective amount of an AD therapy to the subject if the subject is identified as having or at risk of developing AD. In some examples, the AD therapy includes administration of a cholinesterase inhibitor (e.g., galantamine, rivastigmine, or donepezil), administration of an immunotherapy (e.g., a monoclonal antibody targeting beta-amyloid, such as lecanemab or donanemab), administration of an N-methyl-D-aspartate (NMDA) antagonist (e.g., memantine), or administration of brexpiprazole.

Also provided herein are methods of identifying a therapeutic agent for the treatment of Alzheimer's disease (AD). In some aspects, the method includes (i) incubating, in vitro, fibroblast cells or induced neuronal (iN) cells originating from a subject with AD under tissue culture conditions; (ii) contacting the fibroblast cells or iN cells with a test agent; (iii) performing a methylation sequencing assay on genomic DNA isolated from the cells following contact with the test agent to identify a methylation status of one or more of the genomic positions listed in Table 1 and/or Table 2; and (v) identifying the test agent as a therapeutic agent for the treatment of AD if at least one of the genomic positions has a different methylation status compared to control cells not contacted with the test agent; or identifying the test agent as not a therapeutic agent for the treatment of AD if the genomic positions do not have a different methylation status compared to control cells not contacted with the test agent.

The foregoing and other features of this disclosure will become more apparent from the following detailed description of several aspects which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B: Overview of single-nucleus multi-omics to dissect epigenome erosion of LOAD in in vivo entorhinal cortex and in vitro iNs. FIG. 1A: Schematic illustration of generating single-nucleus multi-omics datasets on postmortem entorhinal cortex and fibroblast-derived iNs. FIG. 1B: Design to conduct AD versus CTRL differential analysis and in vivo versus in vitro comparison.

FIGS. 2A-2E: Epigenomic profiling of AD entorhinal cortex with snm3C-seq. FIG. 2A: Iterative clustering and annotation of human brain nuclei. Clustering and visualization by t-distributed stochastic neighbor embedding (t-SNE) were based on mCG and mCH levels at 100 kb bins. Major cell types were annotated and colored based on the integration analysis of methylome datasets from the Human Brain atlas published previously (Tian et al., bioRxiv, 2022.11.30.218285, 2022). FIG. 2B: t-SNE visualization on the clustering colored by groups and individuals. FIG. 2C: t-SNE visualization of gene body mCH levels for cell type marker genes, such as SATB2 (excitatory neurons), SLC6A1 (inhibitory neurons), MEF2C (all neurons), CUX2 (layer 2 and 3 neurons), SULF1 (oligodendrocytes), and RELN (astrocytes and oligodendrocytes). FIG. 2D: The number of cell types, individual fractions and hypomethylated aDMRs. aDMRs bar plot was colored by genome features of the aDMRs located. For example, ASC_AD represents the aDMRs identified in astrocytes and hypomethylated in AD compared to CTRL. FIG. 2D: Heatmap showing the average methylation level at aDMRs in pseudo bulk of major cell types across individuals. The columns were pseudo bulk samples grouped and colored by major cell types, groups (AD or CTRL) and individuals. The rows are hypomethylated aDMRs grouped and colored by finding cell types and hypomethylated groups.

FIGS. 3A-3H: aDMR-enriched hotspots and interaction between aDMRs with repressive promoters of differentially expressed genes (DEGs). FIG. 3A: The distribution of 1,795 aDMR-enriched hotspots across the genomes, chr1-5 shown as examples. 5 kb-bin hotspots were flanked to 1M for visualization and colored by the cell types where hotspots were identified. For each chromosome, the top copy shows the cell type specific hotspots, the lower copy shows the shared hotspots found in at least two brain cell types. FIG. 3B: Genome feature enrichments, Y-axis present log 2 value of fold changes across the genome features (X-axis) between the aDMRs hotspots compared to the genome 5 kb bin background. The star marked the p-value<0.01 in fisher's exact test. FIGS. 3C-3D: Heatmap showing the log 2 value of fold changes of ChromHMM states enrichment for the aDMRs (FIG. 3C) and the promoter of aDMRs linked DEGs in corresponding cell types (FIG. 3D). The star marked p-value <0.01 in Fisher's exact test. FIG. 3E: ChromHMM states enrichment analysis of the selected states (Repressive: EnhBiv, ReprPC, TssBiv; Active: TssA and EnhAl) on the promoter of aDMRs linked DEGs in excitatory neurons over the number of aDMRs linked. FIG. 3F: Venn diagrams showing the overlapping of aDMRs linked DEGs, DEGs harboring promoters in repressive states (TssBiv and ReprPC), and DEGs from published snRNA-seq datasets (Morabito et al., Nature Genetics 53, 1143-1155, 2021). FIG. 3G: Scatter plot of the average log 2 RNA expression fold changes (AD/CTRL) of DEGs as X-axis and average methylation difference (AD-CTRL) of aDMRs linked to corresponding DEGs in excitatory and inhibitory neurons. The size and color represent the number of linked aDMRs and whether the promoter of the DEG is on repressive chromHMM states (TssBiv and ReprPC). FIG. 3H: KEGG pathway enrichment analysis for aDMRs linked DEGs in excitatory neurons. X-axis shows the enrichment significance as −log (adjusted p-value), and Y-axis represents pathways. The size and color represent the number of related genes and combined score, respectively.

FIGS. 4A-4J: Chromosomal epigenome erosion in AD entorhinal cortex. FIG. 4A: Chromatin contact map of AD and CTRL microglia at chromosome 5. FIG. 4B: Frequency of contacts against genomic distance in each single cell of microglial type, Z-score normalized within each cell (column). The y-axis is binned at log 2 scale. Each cell in the x-axis is grouped and colored by individuals and CTRL or AD. FIG. 4C: The log 2 short/long contacts ratio of major types across individuals. Centerline denotes the median, box limits denote the first and third quartiles, and whiskers denote 1.5× the interquartile range. FIG. 4D: Chromosome-wide Pearson's correlation matrix of microglia cells from AD and CTRL. Chr5 as an example. Color bar ranges from −0.16 to 0.21. FIG. 4E: Saddle plots (method) of microglial cells from AD and CTRL shown in (FIG. 4B), colored by contact frequency enrichment showing the interaction of A/B compartment in cis. FIG. 4F: The compartment strength (AA+BB)/(AB+BA) across major cell types between AD (Red) and CTRL individuals (Blue). FIG. 4G: Chromatin conformation around the gene TEME59 shows A to B compartment switching and decreased loop interactions in AD inhibitory neurons. Upper panel, chromatin contact map of AD and CTRL inhibitory neurons with differential loops between AD and CTRL marked in cyan box. Lower panel, A(Red) and B(Blue) compartments track across individuals. FIG. 4H: The total loop interaction numbers of major types across individuals. FIGS. 4I-4J: Illustration of 3D chromatin conformation in normal brain cell types (FIG. 4I) and AD (FIG. 4J).

FIGS. 5A-5F: Single nucleus methylome and transcriptome sequencing (snmCT-seq) characterizing distinct cell states in direct converted neurons from fibroblasts. FIG. 5A: Two-dimensional uniform manifold approximation and projection (UMAP) visualization of snmCT-seq data for fibroblasts and direct-converted iNs. Each dot represents a nucleus colored by annotated cell states. FIG. 5B: Nucleus clustering colored by cell states in AD and CTRL. FIG. 5C: UMAP of nucleus colored by logarithmized counts per million (CPM) of marker gene vimentin (VIM) (fibroblasts), microtubule associated protein 2 (MAP2) (iNs) and two proneuronal factors achaete-scute family bHLH transcription factor 1 (ASCL1) and neurogenin 2 (NGN2). FIG. 5D: The top RNA marker genes in each specific cell state in AD and CTRL. FIG. 5E: Venn diagrams of shared fibroblast->iN conversional DMRs between AD and CTRL. Hypomethylated fibroblast->iN DMRs presenting the DMRs loss of methylation from fibroblasts to iNs and hypermethylated means gain of methylation during iN induction. FIG. 5F: Heatmap showing the average methylation fraction at conversional DMRs in pseudo bulk of fibroblasts and iN cell states across individuals. The columns are pseudo bulk samples grouped and colored by group (AD or CTRL), cell states (fibroblasts and iNs) and individuals. The rows are hypomethylated conversional DMRs grouped and colored by group (AD or CTRL) and cell states (fibroblasts and iNs).

FIGS. 6A-6G: Joint analysis of DNA methylome and RNA identifies DEGs and aDMRs in human fibroblasts and fibroblast-induced neurons. FIGS. 6A-6B: DEGs between AD versus CTRL in fibroblast (FIG. 6A) and iN (FIG. 6B) states, colored by up-(red) or down-regulated (blue) DEGs in AD samples. FIG. 6C: The top up- or down-regulated DEGs in fibroblasts and iNs. FIG. 6D: UpSet plots summarized up- or down-regulated DEGs in fibroblasts and iNs. The bottom left horizontal bar graph shows the total number of DEGs. The top bar graph presents unique or overlapping DEGs. FIG. 6E: KEGG pathway enrichment analysis for up-regulated DEGs in fibroblasts and iNs. X-axis shows the enrichment significance as −log(adjusted p-value), and Y-axis represents pathways. The size and color represent the number of related genes and combined score, respectively. FIG. 6F: Venn diagrams showing the overlapping of aDMRs in both fibroblasts and iNs. For example, hypomethylated aDMRs present the aDMRs hypomethylated in AD whereas hyper aDMRs means hypermethylated in AD samples. FIG. 6G: Heatmap showing the average methylation level at aDMRs in pseudo bulk of fibroblast and iN cell states across individuals. The columns are pseudo bulk samples grouped and colored by cell states (fibroblasts and iNs), groups (AD or CTRL) and individuals. The rows are hypomethylated aDMRs grouped and colored by cell states (fibroblasts and iNs) and group (AD or CTRL).

FIGS. 7A-7G: A methylome-based predictive model captures AD-specific DNA methylation signatures in in vitro fibroblast/iNs and in vivo entorhinal cortex brain cell types. FIG. 7A: Venn diagrams of shared aDMRs between in vitro iN cellular models and in vivo primary entorhinal cortex. FIG. 7B: Heatmap showing the average methylation level at shared aDMRs (1,095 hypomethylated and 1,179 hypermethylated in AD) in pseudo bulk of major cell types including in vivo brain cell types and in vitro fibroblast/iNs across individuals. FIG. 7C: Heatmap showing the average methylation level in isogenic individuals have both in vitro and in vivo cell types at shared aDMRs, and random selected aDMRs identified only from in vitro or in vivo systems (aDMRs number: 2,274 shared, 2,207 random sampling from 160,879 in vitro aDMRs, 1,798 random sampling from 206,239 in vivo aDMRs). FIG. 7D: Design of methylome predictors across in vitro fibroblast/iN and entorhinal cortex tissues. FIG. 7E: The bar plot shows the accuracy of AD predictors used in validation for cross-individuals. FIG. 7F: Heatmap presents the normalized mC level at the selected features of AD predictors across the individuals, groups and cell types. The prediction results were marked as correct in gray and wrong in white.

FIG. 7G: The Pearson coefficient correlation shows the similarity of the pseudo bulk methylation pattern at selected features across individuals, groups and cell types.

FIGS. 8A-8E: QC metrics of snm3C-seq in entorhinal cortex and cell type-specific aDMRs. FIG. 8A: DNA modality QC metrics per cell grouped by individuals on distribution of overall mapping rate, total reads number and global mCG level. FIG. 8B: Chromatin conformation modality QC metrics per cell grouped by individuals on distribution of total cis-contact reads, total trans-contact reads and cis-contact ratio. FIG. 8C: t-SNE visualization of global methylation level at CG, CH and CCC context. FIG. 8D: Examples of cell type-specific aDMRs. FIG. 8E: Scatter plot shows the significant enrichment of motifs (E, −log(p-value) >15) at hypomethylated aDMRs identified in corresponding brain cell types, with the size of the dot indicating the −log(p-value).

FIGS. 9A-9C: DEGs linked by loop interaction with aDMRs. FIG. 9A: 1,795 hotspots of aDMRs across the total autosomes. 5 kb-bin hotspots are flanked to 1M only for visualization and colored by cell types where hotspots are identified from. The left chromosome copy shows the cell type specific hotspots, the right copy shows the hotspots found in at least two brain cell types. FIG. 9B: Scatter plot of the average log 2 RNA expression fold changes (AD/CTRL) of DEGs as X-axis and average methylation difference (AD-CTRL) of DEGs linked aDMRs in glia cell types (astrocytes (ASC), microglial cells (MGC), oligodendrocytes (ODC) and OPC). The size and color represent the number of linked aDMRs and whether the promoter of the gene is on repressive chromHMM states (TssBiv and ReprPC). FIG. 9C: KEGG pathway enrichment analysis for aDMRs linked DEGs in ASC, MGC, ODC and OPC. X-axis shows the enrichment significance as −log (adjusted p-value), and Y-axis represents pathways. The size and color represent the number of related genes and combined score, respectively.

FIGS. 10A-10E: Chromosomal epigenome erosion occurs in multiple cell types. FIG. 10A: Chromatin contact map of excitatory neurons (Ex), inhibitory neurons (Inh), astrocytes (ASC) and oligodendrocytes (ODC) from AD and CTRL across chromosomes, chr5 as an example. FIG. 10B: Frequency of contacts against genomic distance in each single cell of Ex, Inh, ASC and ODC cell types, Z-score normalized within each cell (column). The y-axis is binned at log 2 scale. Each cell in the x-axis is grouped and colored by individuals and CTRL or AD. FIG. 10C: The log 2 ratio between AD and CTRL of frequency of contacts grouped by genomic distance and the difference of raw compartment scores at the two anchors of a contact across cell types. FIG. 10D: Contact scores between the intra-compartment, inter-compartment regions and the ratio between inter- and intra-compartment interactions. FIG. 10E: Contacts correlation around domain boundaries of major types across individuals.

FIGS. 11A-11J: Fibroblast iNs retain aging-related methylation signatures compared to iPSCs derived iNs. FIG. 11A: Schematic representation of comparing iPSC-differentiated iNs and fibroblast directly converted iNs via inducible expression of the same proneuronal factors NGN2-2A-ASCL1 (N2A). FIGS. 11B-11D: Clustering and annotation of iNs generated via two methods based on methylation levels of 100 kb bins. Cells were colored by samples (FIG. 11B), starter cell sources (FIG. 11C), and collection days (FIG. 11D). FIGS. 11E-11F: Global methylation levels of mCH (FIG. 11E) and mCG (FIG. 11F) on 2D UMAP clustering. (FIGS. 11G-11H) Violin plots showing the average levels of mCG (FIG. 11G) and mCH (FIG. 11H) across donor and cell types. FIG. 11I. The genome coverage consists of partially methylated domains (PMDs) across cell types. FIG. 11J: DNA methylation (DNAm) age prediction of fibroblast/iNs and iPSC/iNs by multi-tissue age estimator (Horvath, Genome Biol. 14, 2013) compared to chronological age.

FIGS. 12A-12I: snmCT-seq quality control (QC) metrics and cell state annotation. FIG. 12A: DNA modality QC metrics per cell grouped by individuals on distribution of total mapping rate, total reads number and genome coverages. FIG. 12B: RNA modality QC metrics per cell grouped by individuals on distribution of total RNA reads, gene detected, and mitochondria read ratio. FIG. 12C: UMAP embedding before and after batch correction of mCG and mCH level at 100 kb bins. Cells were colored by individuals, groups and collection day separately. FIG. 12D: UMAP embedding before and after batch correction of RNA expression. Cells were colored by individuals, groups and collection day separately. FIGS. 12E-12F: Cell number distribution of collection day (FIG. 12E) and cell states on Day 21 (FIG. 12F) across individuals. FIG. 12G: Genomic feature fraction of conversional DMRs grouped by hypermethylated or hypomethylated between fibroblasts versus iNs in AD and CTRL groups. FIGS. 12H-12I: Scatter plot shows the enrichment of transcription factor (TF) motifs (FIG. 12H) at hypomethylated conversional DMRs in iNs compared to fibroblasts in AD and CTRL, with the size of the dot indicating the −log(p-value). The corresponding RNA expression of TF candidates is shown in FIG. 12I, the size of dot presents the fraction of expressed cells whereas colors show the mean expression (log (CPM+1)) of cell populations.

FIGS. 13A-13E: Motif enrichment analysis of aDMRs and their linked DEGs in in vitro cellular models. FIGS. 13A-13B: Scatter plot shows the motif enrichment analysis of the hypomethylated aDMRs in AD and CTRL in fibroblasts/iNs states, with the size of the dot indicating the −log(p-value). The corresponding RNA expression of TF candidates is shown in FIG. 13B, the size of dot presents the fraction of expressed cells whereas colors show the mean expression (log (CPM+1)) of cell populations. FIG. 13C: Venn diagrams showing the overlapping of shared DEGs identified in fibroblast with DEGs linked with aDMRs by GREAT algorithm (McLean et al., Nat. Biotechnol.28, 2010). FIG. 13D: RNA expression scatter of DEGs in fibroblasts (upper panel) and iN (lower panel) with linked aDMRs by GREAT algorithm. X axis and Y axis present log 2 value of normalized gene expression as CPM (counts per million) in AD and CTRL samples. The size and color represent the number of linked aDMRs and average methylation changes, respectively. Top DEG names are labeled beside the dot. FIG. 13E: The distribution of DEGs number in fibroblast and iN over the minimal number of aDMRs associated by GREAT algorithm, colored by the Pearson correlation coefficient between log 2 fold changes of RNA expression (AD/CTRL) and average methylation difference (AD-CTRL) of associated aDMRs by GREAT algorithm.

FIGS. 14A-14C: Examples of machine learning (ML) selected features in AD predictors. FIG. 14A: Top 3 motif enrichment of shared aDMRs. FIG. 14B: Datasets used for training and testing of AD predictors. FIG. 14C: De novo selective AD predictors by machine learning are overlapped with shared aDMRs across cell types. Upper panel, chromatin conformation around the gene BCL6 shows the decreased loop contacts in AD microglia. Middle panels, the selected features in the AD predictor are located at LINC01991, TPRG1 and P3H2 intronic regions, overlapped with shared aDMRs across cell types. Lower panel, browser view of methylation level surrounds the AD predictors in AD and CTRL.

### DETAILED DESCRIPTION

### I. Introduction

To characterize genome-wide AD-specific methylation signatures from in vivo brain cell types, single-nucleus methyl-3C sequencing (sn-m3C-seq) was performed to jointly profile chromatin conformation and methylome from the same cell. This approach enabled the definition of the cell type taxonomy in AD patients and identified differentially methylated regions between AD and control (aDMRs) within and across brain cell types and revealing erosion of the epigenome in single brain cells of AD patients based on cell type-specific 3D genome structure alterations.

In addition, to assess whether the epigenetic signatures found from in vivo human brain tissues can be detected in cellular models, induced neurons (iNs) were directly converted from dermal fibroblasts of AD patients and snmCT-seq datasets capturing transcriptome and methylome of fibroblasts and iNs were generated. The distinct cell states of in vitro cellular iN models were defined and epigenetic signatures of AD from age-retaining iNs were characterized. A comparative analysis between in vitro cellular models and in vivo primary brain tissues identified conserved and robust methylation signatures.

A reliable set of machine learning model selected CpG sites showed extremely high accuracy of AD prediction across in vitro and in vivo cell types.

### II. Abbreviations

AD Alzheimer's disease
aDMR AD differentially methylated region
ASC astrocytes
ASCL1 achaete-scute family bHLH transcription factor 1
cDMR conversion-related differentially methylated region
CpG cytosine-guanine dinucleotide
CPM counts per million
CRE cis-regulatory element
CTRL control
CUX2 cut like homeobox 2
DEG differentially expressed gene
DNAm DNA methylation
iN cell induced neuronal cell
INP intermediate neuronal progenitor
iPSC induced pluripotent stem cell
LOAD late onset Alzheimer's disease
m5C methylated cytosine
MAP2 microtubule associated protein 2
mCG methylated cytosine-guanine
mCH methylated cytosine-adenine/cytosine/thymine
MEF2C myocyte enhancer factor 2C
MET mesenchymal to epithelial transition
MGC microglial cell
ML machine learning
NGN2 neurogenin 2
NGS next generation sequencing
NMDA N-methyl-D-aspartate
ODC oligodendrocytes
OPC oligodendrocyte progenitor cell
PMD partially methylated domain
QC quality control
RELN reelin
SATB2 SATB homeobox 2
SLC6A1 solute carrier family 6 member 1
SULF1 sulfatase 1
t-SNE t-distributed stochastic neighbor embedding
TAD topologically associating domain
TF transcription factor
UMAP uniform manifold approximation and projection
VIM vimentin

### III. Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context indicates otherwise. For example, the term “a cell” includes single or plural cells and can be considered equivalent to the phrase “at least one cell.” As used herein, the term “comprises” means “includes.” Unless otherwise indicated “about” indicates within five percent. It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. To facilitate review of the various embodiments, the following explanations of terms are provided:

Administration: The introduction of a composition (such as one containing an agent that prevents or treats a brain disorder) into a subject by a chosen route. Administration can be local or systemic. For example, if the route is intravenous, the composition is administered by introducing the composition into a vein of the subject. Similarly, if the route is intramuscular, the composition is administered by introducing the composition into a muscle of the subject. If the chosen route is oral, the composition is administered by ingesting the composition.

Exemplary routes of administration of use in the methods disclosed herein include, but are not limited to, oral, injection (such as subcutaneous, intramuscular, intradermal, intraperitoneal, intraosseous, and intravenous), sublingual, rectal, transdermal (for example, topical), intranasal, vaginal, and inhalation routes. Administration can also be local, such as to the brain of a subject.

An active agent, as used herein, is a drug, medicament, pharmaceutical, therapeutic agent, nutraceutical, or other compound that may be administered to the lungs. The active agent may be a “small molecule,” generally having a molecular weight of about 2000 daltons or less. The active agent may also be a “biological active agent.” Biological active agents include proteins, antibodies, antibody fragments, peptides, oligonucleotides, vaccines, and various derivatives of such materials.

Alzheimer's disease (AD): People with AD experience memory loss and cognitive difficulties. Most people with AD have late-onset Alzheimer's disease (LOAD), in which symptoms become apparent in their mid-60s. Early-onset Alzheimer's disease occurs between a person's 30s to mid-60s and represents less than 10 percent of all people with Alzheimer's.

AD therapy: A treatment administered to a patient diagnosed as having or at risk of developing AD that prevents, inhibits, or relieves AD in the patient. An AD therapy includes, for example, administration of a large- or small-molecule drug, a gene therapy, or a physical or mental therapy to the patient to treat AD. In some aspects, the therapy includes administration of antibodies/immunotherapies (e.g., a monoclonal antibody targeting beta-amyloid, such as lecanemab donanemab, or aducanumab), cholinesterase inhibitors (such as donepezil, rivastigmine, and galantamine), brexpiprazole, and/or NMDA antagonists (such as memantine).

Biological sample: A biological sample contains genomic DNA, RNA (including mRNA), protein, or combinations thereof, which can be obtained from a subject, such as a human. Examples include, but are not limited to, sputum, saliva, mucus, nasal wash, peripheral blood, tissue (such as brain tissue), cells, urine, tissue biopsy (such as skin biopsy), fine needle aspirate, surgical specimen, feces, cerebral spinal fluid (CSF), synovial fluid, bronchoalveolar lavage (BAL) fluid, nasopharyngeal samples, oropharyngeal samples, and autopsy material. In some aspects, biological samples are cells directly obtained from a subject, such as brain cells and fibroblasts; in other aspects, biological samples are cells derived from cells directly obtained from a subject, such as induced neuronal cells.

Bisulfite treatment: The treatment of DNA with bisulfite or a salt thereof, such as sodium bisulfite (NaHSO₃). Bisulfite reacts readily with the 5,6-double bond of cytosine, but poorly with methylated cytosine. Cytosine reacts with the bisulfite ion to form a sulfonated cytosine reaction intermediate which is susceptible to deamination, giving rise to a sulfonated uracil. The sulfonate group can be removed under alkaline conditions, resulting in the formation of uracil. Uracil is recognized as a thymine by polymerases and amplification will result in an adenine-thymine base pair instead of a cytosine-guanine base pair.

Control: A sample or standard used for comparison with an experimental sample. In some aspects, the control is a sample obtained from a healthy subject (such as a subject without AD and other cognitive diseases); in other aspects, the control is a sample obtained from an AD subject. In some aspects, the control subject (healthy or AD) is age-matched to the subject providing the experimental sample, which means the control subject is around the same age (±5 years old) as the subject providing the experimental sample. In some aspects, the control is a historical control or standard reference value or range of values (such as a previously tested control sample, such as the methylation status of a target nucleic acid or particular CpG site in a subject without AD and other cognitive diseases, or the methylation status of a target nucleic acid or particular CpG site in an AD subject). As used herein, a normal control is a sample or standard from or based on a subject without AD and other cognitive diseases; an AD control is a sample or standard from or based on a subject diagnosed with AD. In some examples, the controls are age-matched.

CpG Site: A di-nucleotide DNA sequence comprising a cytosine followed by a guanine in the 5′ to 3′ direction. The cytosine nucleotides of CpG sites in genomic DNA are the target of intracellular methyltransferases and can have a methylation status of methylated or not methylated. Reference to “methylated CpG site” or similar language refers to a CpG site in genomic DNA having a 5-methylcytosine (5mC) nucleotide.

Fibroblast: A type of cell that contributes to the formation of connective tissue, a fibrous cellular material that supports and connects other tissues or organs in the body.

Genome/genomic: All of the genetic material in the chromosomes of an organism. DNA derived from the genetic material in the chromosomes of a particular organism is genomic DNA.

Genomic segment: A contiguous sequence of genomic DNA no more than 2000 bases in length. Genomic position refers to the position of a nucleotide within the genomic segment.

Induced neuronal (iN) cell: Neurons derived from somatic cells by reprogramming somatic cells to neurons. In some aspects, iNs are derived from fibroblasts. In some aspects, iNs are directly converted from fibroblasts without the fibroblasts going through a stem cell intermediate phase. This term is used interchangeably with induced neurons.

Methylation: The addition of a methyl group (—CH₃) to cytosine nucleotides of CG or CH (H=A, C, or T) sites in DNA. DNA methylation, the addition of a methyl group onto a nucleotide, is a post-replicative covalent modification of DNA that is catalyzed by a DNA methyltransferase enzyme. In biological systems, DNA methylation can serve as a mechanism for changing the structure of DNA without altering its coding function or its sequence.

Methylation percentage: The percentage of methylated cytosine detected at a CpG site among a plurality of sequence reads covering the site obtained from a methylation sequencing assay. For example, if 100 sequence reads are obtained, and 90 sequence reads show a methylated cytosine at a CpG site, then the methylation percentage for that site is 90%. When comparing two methylation percentages, a first percentage is said to be different from a second percentage by at least X %, if compared to the second percentage, the first percentage is increased or decreased by at least X %. For example, if the second percentage is 50% and the first percentage is anywhere between 0-45% or 55-100%, then the first percentage differs from the second by at least 5%.

Methylation sequencing assay: A sequencing assay that detects the methylation status of one or more CpG and/or CH sites in DNA. A non-limiting example of a methylation sequencing assay is a sequencing assay performed on bisulfite-treated and amplified genomic DNA. Many approaches leverage the high quality and sensitivity of next-generation sequencing (NGS) for methylation analysis. Most methods rely on bisulfite conversion of DNA to detect unmethylated cytosines. Bisulfite conversion changes unmethylated cytosines to uracil during library preparation. Converted bases are identified (after PCR) as thymine in the sequencing data, and read counts are used to determine the % methylated cytosines. Bisulfite conversion sequencing can be done with targeted methods such as amplicon methyl-seq or target enrichment, or with whole-genome bisulfite sequencing. Additionally, alternative chemistries like OxBS and TAB-Seq can be used with NGS for identification of hydroxymethylation (5-hMc) in conjunction with methylation (5-mc) analysis.

Methylation status: The status of methylation (methylated or not methylated) of a cytosine nucleotide within a genomic sequence. In some aspects, the cytosine nucleotide is part of a CpG site.

Methylation marker/signature: A cytosine nucleotide (in a CpG or CH) in a genome, that has a different methylation status according to the presence or absence of a disease, such as AD. Methylation typically occurs in a CpG containing nucleic acid. The CpG containing nucleic acid may be present in, e.g., a CpG island, a CpG doublet, a promoter, an intron, or an exon of gene. In some aspects, cytosines within a region plus or minus 200, 150, 100, 90, 80, 70, 60, 50, 45, 40, 35, 30, 25, 20, 15, 10, or 5 bases of a methylation marker (such as those listed in Tables 1 and 2) in a genome can also be used to distinguish AD and non-AD subjects.

Methylome: The information of DNA methylation of all cytosines in a genome.

Sequence Read: A sequence (e.g., of about 300 bp) of contiguous base pairs of a nucleic acid molecule. The sequence read may be represented symbolically by the base pair sequence (in ATCG) of the sample portion. In some aspects, the sequence read includes methylation information of cytosines. A sequence read may be stored in a memory device and processed as appropriate to determine whether it matches a reference sequence or meets other criteria. A sequence read may be obtained directly from a sequencing apparatus or indirectly from stored sequence information concerning a sample.

Subject: A living multi-cellular vertebrate organism, a category that includes human and non-human mammals.

Test agent: A potential active agent for having a therapeutic effect on a disease, such as AD.

Therapeutically effective amount: A quantity of a compound to achieve a desired effect in a subject being treated. For instance, this can be the amount necessary to treat or prevent a brain disease, such as AD, particularly LOAD. When administered to a subject, a dosage will generally be used that will achieve target tissue concentrations that have been shown to achieve an in vitro effect. A therapeutically effective amount may vary depending upon one or more of: the subject and disease condition being treated, the weight and age of the subject, the severity of the brain disease, the manner of administration and the like, which can readily be determined by one of ordinary skill in the art. The beneficial therapeutic effect can include enablement of diagnostic determinations; amelioration of the brain disease symptoms, improvement of brain function, reducing or preventing the onset of brain disease symptoms. In one aspect, an “effective amount” is an amount sufficient to reduce symptoms of a brain disease, for example by at least 10%, at least 20%, at least 50%, at least 70%, at least 90%, at least 95%, or even 100% (as compared to no administration of the therapeutic agent), or that delays onset or progression.

Treating or treatment: With respect to disease, either term includes (1) preventing the disease, e.g., causing the clinical symptoms of the disease not to develop in an subject that may be exposed to or predisposed to the disease but does not yet experience or display symptoms of the disease, (2) inhibiting the disease, e.g., arresting the development of the disease or its clinical symptoms, or (3) relieving the disease, e.g., causing regression of the disease or its clinical symptoms.

Tissue culture conditions: Standard tissue culture conditions appropriate for the types of cells being cultured.

### IV. Identifying Subjects Having or at Risk of Developing Alzheimer's Disease

Provided herein are methods of identifying a subject as having or at risk of developing Alzheimer's disease (AD). In some aspects, the method includes obtaining sequence reads of a methylation sequencing assay covering genomic segments of a biological sample from the subject, wherein the genomic segments contain one or more of the genomic positions listed in Table 1 and/or Table 2; and identifying the subject as having or at risk of developing AD if at least one of the genomic positions has a different methylation status compared to a normal control, or identifying the subject as not having or at risk of developing AD if none of the genomic positions has a different methylation status compared to a normal control.

In some aspects, the one or more of the genomic positions listed in Table 1 and/or Table 2 are at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, about 190, at least 200, at least 225, at least 250, at least 275 or at least 300 of the genomic positions listed in Table 1 and/or Table 2.

In some examples, the one or more genomic positions are the 300 genomic positions listed in Table 2, or a subset of at least 20, at least 40, at least 60, at least 80, at least 100, at least 120, at least 140, at least 160, at least 180, at least 200, at least 220, at least 260, or at least 280 thereof. In particular examples, the one or more genomic positions are the genomic positions ranked 1-20, 1-40, 1-60, 1-80, 1-100, 1-120, 1-140, 1-160, 1-180, 1-200, 1-220, 1-240, 1-260 or 1-280 in Table 2.

In some examples, the one or more genomic positions are selected from those listed in Table 3, which includes chr3: 107351515-107351516; chr1: 169668153-169668154; chr9: 114150865-114150866; chr10: 77298787-77298788; chr1: 218669424-218669425; chr18: 7393790-7393791; chr16: 85241293-85241294; chr2: 78006878-78006879; chr19: 49468339-49468340; chr2: 171599550-171599551; chr2: 38079793-38079794; chr13: 29714764-29714765; chr2: 223827178-223827179; chr13: 29697391-29697392; chr2: 223823348-223823349; chr13: 66231576-66231577; chr4: 112701336-112701337; chr2: 54341024-54341025; chr2: 223389403-223389404; and chr2: 54323871-54323872. In specific examples, the one or more genomic positions consist of the 20 genomic positions listed in Table 3.

In some aspects, the one or more genomic segments are up to 300 bases upstream (also referred to as “minus” or “5′”) or up to 300 bases downstream (also referred to as “plus” or “3′”) of the genomic positions listed in Table 1, Table 2 and/or Table 3, such as about 50 to about 300 bases upstream or downstream of the listed genomic positions. In some examples, the genomic segments are up to about 275 bases, 250 bases, 225 bases, 200 bases, 175 bases, 150 bases, 125 bases, 100 bases, 75 bases, 50 bases, or 25 bases upstream or up to about 275 bases, 250 bases, 225 bases, 200 bases, 175 bases, 150 bases, 125 bases, 100 bases, 75 bases, 50 bases, or 25 bases downstream of the genomic positions listed in Table 1, Table 2 and/or Table 3.

In some aspects, the method includes obtaining methylome data for the entire biological sample, such as the complete methylome data (the DNA methylation status of all cytosines in a genome) for a single cell or a plurality of cells.

In some aspects, the biological sample is a single cell. In some examples, the single cell is a single fibroblast cell. In other examples, the single cell is a single induced neuronal (iN) cell. In particular examples, the iN cell is directly converted from a fibroblast cell without going through a stem cell intermediate phase.

In some aspects, the biological sample includes a plurality of cells. In some examples, the plurality of cells is a plurality of fibroblast cells. In other examples, the plurality of cells is a plurality of iN cells. In particular examples, the iN cells are directly converted from fibroblast cells without going through a stem cell intermediate phase.

In some aspects, the method further includes obtaining the biological sample from the subject. In some examples, the biological sample is obtained by skin biopsy. In particular examples, a fibroblast cell or fibroblast cells are obtained from the skin biopsy and is/are converted into an iN cell or iN cells.

In some aspects, the methylation sequencing assay is a bisulfite sequencing assay. Other methylation sequencing methods can be utilized, such as a method described in section VI.

In some aspects, the AD is late-onset AD (LOAD).

In some aspects, the method further includes administering a therapeutically effective amount of an AD therapy to the subject if the subject is identified as having or at risk of developing AD. In some examples, the AD therapy includes administration of a cholinesterase inhibitor (e.g., galantamine, rivastigmine, or donepezil), administration of an immunotherapy (e.g., a monoclonal antibody targeting beta-amyloid, such as lecanemab or donanemab), administration of an N-methyl-D-aspartate (NMDA) antagonist (e.g., memantine), administration of brexpiprazole, or any combination thereof.

In some aspects, the method further includes calculating a methylation fraction for each of the genomic positions. In some examples, the genomic position of the subject has a different methylation status compared to the normal control if the methylation fraction of the subject is different from the normal control by at least 5%, 10%, 15%, 20%, 25%, 30%, 35%, 40%, 45%, 50%, 55%, 60%, 65%, 70%, 75%, or 80%.

### V. Identifying Therapeutic Agents for the Treatment of Alzheimer's Disease

Also provided herein are methods of identifying a therapeutic agent for the treatment of Alzheimer's disease (AD). In some aspects, the method includes (i) incubating, in vitro, fibroblast cells or induced neuronal (iN) cells originating from a subject with AD under tissue culture conditions; (ii) contacting the fibroblast cells or iN cells with a test agent; (iii) performing a methylation sequencing assay on genomic DNA isolated from the cells following contact with the test agent to identify a methylation status of one or more of the genomic positions listed in Table 1 and/or Table 2; and (v) identifying the test agent as a therapeutic agent for the treatment of AD if at least one of the genomic positions has a different methylation status compared to control cells not contacted with the test agent; or identifying the test agent as not a therapeutic agent for the treatment of AD if the genomic positions do not have a different methylation status compared to control cells not contacted with the test agent.

In some aspects, the one or more of the genomic positions listed in Table 1 and/or Table 2 are at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, about 190, at least 200, at least 225, at least 250, at least 275 or at least 300 of the genomic positions listed in Table 1 and/or Table 2.

In some examples, the one or more genomic positions are the 300 genomic positions listed in Table 2, or a subset of at least 20, at least 40, at least 60, at least 80, at least 100, at least 120, at least 140, at least 160, at least 180, at least 200, at least 220, at least 260, or at least 280 thereof. In particular examples, the one or more genomic positions are the genomic positions ranked 1-20, 1-40, 1-60, 1-80, 1-100, 1-120, 1-140, 1-160, 1-180, 1-200, 1-220, 1-240, 1-260 or 1-280 in Table 2.

In some examples, the one or more genomic positions are selected from those listed in Table 3, which includes chr3: 107351515-107351516; chr1: 169668153-169668154; chr9: 114150865-114150866; chr10: 77298787-77298788; chr1: 218669424-218669425; chr18: 7393790-7393791; chr16: 85241293-85241294; chr2: 78006878-78006879; chr19: 49468339-49468340; chr2: 171599550-171599551; chr2: 38079793-38079794; chr13: 29714764-29714765; chr2: 223827178-223827179; chr13: 29697391-29697392; chr2: 223823348-223823349; chr13: 66231576-66231577; chr4: 112701336-112701337; chr2: 54341024-54341025; chr2: 223389403-223389404; and chr2: 54323871-54323872. In specific examples, the one or more genomic positions consist of the 20 genomic positions listed in Table 3.

In some aspects, the one or more genomic segments are up to 300 bases upstream (also referred to as “minus” or “5′”) or up to 300 bases downstream (also referred to as “plus” or “3′”) of the genomic positions listed in Table 1, Table 2 and/or Table 3, such as about 50 to about 300 bases upstream or downstream of the listed genomic positions. In some examples, the genomic segments are up to about 275 bases, 250 bases, 225 bases, 200 bases, 175 bases, 150 bases, 125 bases, 100 bases, 75 bases, 50 bases, or 25 bases upstream or up to about 275 bases, 250 bases, 225 bases, 200 bases, 175 bases, 150 bases, 125 bases, 100 bases, 75 bases, 50 bases, or 25 bases downstream of the genomic positions listed in Table 1, Table 2 and/or Table 3.

In some aspects, the method includes obtaining methylome data for the entire biological sample, such as the complete methylome data (the DNA methylation status of all cytosines in a genome) for a single fibroblast or iN cell or a plurality of fibroblast or iN cells.

In some aspects, the fibroblast cells are obtained from a skin biopsy from a subject with AD.

In some aspects, the iN cells are directly converted from fibroblast cells obtained from a subject with AD without going through a stem cell intermediate phase.

In some aspects, the methylation sequencing assay is a bisulfite sequencing assay. Other methylation sequencing methods can be utilized, such as a method described in section VI.

In some aspects, the AD is late-onset AD (LOAD).

In some aspects, the method further includes calculating a methylation fraction for each of the genomic positions. In some examples, the genomic position of the subject has a different methylation status compared to the normal control if the methylation fraction of the subject is different from the normal control by at least 5%, 10%, 15%, 20%, 25%, 30%, 35%, 40%, 45%, 50%, 55%, 60%, 65%, 70%, 75%, or 80%.

### VI. Measuring Methylation Status

The methylation status at one or more genomic positions that are associated with AD (DNA methylation markers for AD), such as those disclosed herein, are assayed. DNA methylation markers for AD that can be evaluated are provided in Tables 1-3. In one example, an Illumina™ DNA methylation array is used. In another example, a PCR protocol using relevant primers is utilized. Determining the methylation status of a particular DNA methylation marker can include determining whether a particular region in the genome is methylated or not.

DNA methylation status can be determined using any suitable assay. In one example, a molecular break light assay for DNA adenine methyltransferase activity is used. This assay is based on the specificity of the restriction enzyme DpnI for fully methylated (adenine methylation) GATC sites in an oligonucleotide labeled with a fluorophore and quencher. The adenine methyltransferase methylates the oligonucleotide making it a substrate for DpnI. Cutting of the oligonucleotide by DpnI gives rise to a fluorescence increase, thus indicating that the position is methylated. In one example, methylation-specific polymerase chain reaction (PCR) is used. This method is based on a chemical reaction of sodium bisulfite with DNA that converts unmethylated cytosines of CpG dinucleotides to uracil or UpG, followed by traditional PCR. However, methylated cytosines are not converted in this process, and thus primers are designed to overlap the CpG site of interest, which allows one to determine methylation status as methylated or unmethylated. In one example, whole genome bisulfite sequencing, also known as BS-Seq, is used. This is a genome-wide analysis of DNA methylation based on the sodium bisulfite conversion of genomic DNA, which is then sequenced. The sequences obtained are then re-aligned to the reference genome to determine methylation states of CpG dinucleotides based on mismatches resulting from the conversion of unmethylated cytosines into uracil. In one example, the HpaII tiny fragment Enrichment by Ligation-mediated PCR (HELP) assay is used, which is based on restriction enzymes' differential ability to recognize and cleave methylated and unmethylated CpG DNA sites. In one example, methyl sensitive southern blotting is used, which uses Southern blotting techniques to probe gene-specific differences in methylation using restriction digests. This method can be used to evaluate local methylation near the binding site for the probe. In one example, ChIP-on-chip assay is used. This method uses commercially prepared antibodies to bind to DNA methylation-associated proteins like MeCP2. In one example, restriction landmark genomic scanning is used, which is based upon restriction enzymes' differential recognition of methylated and unmethylated CpG sites. In one example, methylated DNA immunoprecipitation (MeDIP) is used. Immunoprecipitation is used to isolate methylated DNA fragments for input into DNA detection methods such as DNA microarrays (MeDIP-chip) or DNA sequencing (MeDIP-seq). In one example, pyrosequencing of bisulfite treated DNA is used. In this method, an amplicon is generated by a normal forward primer but a biotinylated reverse primer to PCR the target methylation marker. A pyrosequencer then analyzes the sample by denaturing the DNA and adding one nucleotide at a time to the mix according to a sequence given by the user. If there is a mismatch, it is recorded and the percentage of DNA for which the mismatch is present is noted. This provides a percentage methylation per CpG island.

In some examples, the genomic DNA to be analyzed is used directly, e.g., hybridized to a complimentary sequence (e.g., a synthetic polynucleotide sequence) that is attached to a solid support (e.g., one disposed within a microarray). In some examples, the genomic DNA to be analyzed is amplified by a PCR process. For example, prior to or concurrent with hybridization to an array, the sample may be amplified by a variety of mechanisms, such as those that employ PCR. The sample may be amplified on the array.

### VII. Methylation Markers for Alzheimer's Disease

Table 1 lists the chromosomal positions identified as methylation sites for AD prediction. Smaller subsets of the methylation sites are listed in Tables 2 and 3. Table 2 provides a ranked list of 300 of the methylation sites. Table 3 includes 20 of the identified methylation sites. All of the genome coordinates of the markers in Tables 1, 2 and 3 represent CG dinucleotide sites (CpGs) based on human genome assembly GRCh38 (also known as hg38).

TABLE 1
Chromosomal positions of methylation sites correlated with AD

| | | |
|---|---|---|
| chr1: 915809-915810 | chr15: 58474236-58474237 | chr3: 190046403-190046404 |
| chr1: 21380089-21380090 | chr15: 67901611-67901612 | chr3: 192505947-192505948 |
| chr1: 22225255-22225256 | chr15: 67922017-67922018 | chr3: 192868127-192868128 |
| chr1: 22460164-22460165 | chr15: 67928739-67928740 | chr3: 192873527-192873528 |
| chr1: 22526647-22526648 | chr15: 69236063-69236064 | chr3: 194517661-194517662 |
| chr1: 25000294-25000295 | chr15: 70372498-70372499 | chr4: 2451949-2451950 |
| chr1: 26303390-26303391 | chr15: 72856034-72856035 | chr4: 2466024-2466025 |
| chr1: 26572634-26572635 | chr15: 72934907-72934908 | chr4: 2811118-2811119 |
| chr1: 34142939-34142940 | chr15: 80253710-80253711 | chr4: 3759619-3759620 |
| chr1: 37642428-37642429 | chr15: 80277176-80277177 | chr4: 4281948-4281949 |
| chr1: 38585010-38585011 | chr15: 82227042-82227043 | chr4: 4284517-4284518 |
| chr1: 39155880-39155881 | chr15: 84743786-84743787 | chr4: 4286119-4286120 |
| chr1: 41384201-41384202 | chr15: 86862172-86862173 | chr4: 7422413-7422414 |
| chr1: 47442109-47442110 | chr15: 91594542-91594543 | chr4: 21511949-21511950 |
| chr1: 49348753-49348754 | chr15: 91944750-91944751 | chr4: 21781604-21781605 |
| chr1: 53069805-53069806 | chr15: 94817835-94817836 | chr4: 26052795-26052796 |
| chr1: 53598264-53598265 | chr15: 100504533-100504534 | chr4: 42346087-42346088 |
| chr1: 53640709-53640710 | chr16: 1129161-1129162 | chr4: 43994250-43994251 |
| chr1: 53642438-53642439 | chr16: 1257295-1257296 | chr4: 44016182-44016183 |
| chr1: 54393238-54393239 | chr16: 11519017-11519018 | chr4: 61426568-61426569 |
| chr1: 54768847-54768848 | chr16: 12541696-12541697 | chr4: 61430085-61430086 |
| chr1: 54775150-54775151 | chr16: 12558625-12558626 | chr4: 74338400-74338401 |
| chr1: 62337389-62337390 | chr16: 12572421-12572422 | chr4: 78332903-78332904 |
| chr1: 68275770-68275771 | chr16: 17928367-17928368 | chr4: 81715868-81715869 |
| chr1: 79768860-79768861 | chr16: 20668884-20668885 | chr4: 84622305-84622306 |
| chr1: 79779556-79779557 | chr16: 25785755-25785756 | chr4: 88076723-88076724 |
| chr1: 79796616-79796617 | chr16: 30826275-30826276 | chr4: 88216166-88216167 |
| chr1: 79834373-79834374 | chr16: 56970976-56970977 | chr4: 88218680-88218681 |
| chr1: 79919480-79919481 | chr16: 61942889-61942890 | chr4: 88244342-88244343 |
| chr1: 79935157-79935158 | chr16: 78048783-78048784 | chr4: 101155825-101155826 |
| chr1: 79960624-79960625 | chr16: 78048848-78048849 | chr4: 101171837-101171838 |
| chr1: 95836889-95836890 | chr16: 78049572-78049573 | chr4: 101181888-101181889 |
| chr1: 103529898-103529899 | chr16: 81844042-81844043 | chr4: 101185199-101185200 |
| chr1: 112035116-112035117 | chr16: 84777147-84777148 | chr4: 109229536-109229537 |
| chr1: 112039744-112039745 | chr16: 84853958-84853959 | chr4: 112701336-112701337 |
| chr1: 112410114-112410115 | chr16: 85241293-85241294 | chr4: 117419337-117419338 |
| chr1: 112615666-112615667 | chr16: 85241649-85241650 | chr4: 117420472-117420473 |
| chr1: 117786033-117786034 | chr16: 87353981-87353982 | chr4: 120440798-120440799 |
| chr1: 118007821-118007822 | chr16: 88040234-88040235 | chr4: 120597504-120597505 |
| chr1: 118011374-118011375 | chr16: 88158339-88158340 | chr4: 125060744-125060745 |
| chr1: 118608933-118608934 | chr16: 88654269-88654270 | chr4: 128981100-128981101 |
| chr1: 118839603-118839604 | chr17: 744945-744946 | chr4: 129022481-129022482 |
| chr1: 118865664-118865665 | chr17: 749696-749697 | chr4: 129134196-129134197 |
| chr1: 119095484-119095485 | chr17: 3546515-3546516 | chr4: 129331940-129331941 |
| chr1: 119183000-119183001 | chr17: 5941938-5941939 | chr4: 129338021-129338022 |
| chr1: 147032287-147032288 | chr17: 5956533-5956534 | chr4: 129496402-129496403 |
| chr1: 147723335-147723336 | chr17: 9056363-9056364 | chr4: 138306908-138306909 |
| chr1: 156519750-156519751 | chr17: 11335771-11335772 | chr4: 140129688-140129689 |
| chr1: 156535269-156535270 | chr17: 13243728-13243729 | chr4: 141309452-141309453 |
| chr1: 163782298-163782299 | chr17: 18167446-18167447 | chr4: 161118385-161118386 |
| chr1: 164209339-164209340 | chr17: 29410803-29410804 | chr4: 165007102-165007103 |
| chr1: 168588906-168588907 | chr17: 29426599-29426600 | chr4: 172859547-172859548 |
| chr1: 168593667-168593668 | chr17: 32958992-32958993 | chr4: 172879114-172879115 |
| chr1: 169022201-169022202 | chr17: 32964779-32964780 | chr4: 184021965-184021966 |
| chr1: 169668153-169668154 | chr17: 33212412-33212413 | chr4: 184882579-184882580 |
| chr1: 171891978-171891979 | chr17: 66695786-66695787 | chr4: 187572796-187572797 |
| chr1: 188138811-188138812 | chr17: 67361575-67361576 | chr4: 188849107-188849108 |
| chr1: 188259784-188259785 | chr17: 76392900-76392901 | chr4: 188852114-188852115 |
| chr1: 188266557-188266558 | chr17: 78274971-78274972 | chr4: 188862889-188862890 |
| chr1: 188422259-188422260 | chr17: 78293563-78293564 | chr5: 718776-718777 |
| chr1: 188544584-188544585 | chr17: 79448950-79448951 | chr5: 1184728-1184729 |
| chr1: 192557734-192557735 | chr17: 79817698-79817699 | chr5: 3240157-3240158 |
| chr1: 193522116-193522117 | chr17: 80680543-80680544 | chr5: 4923693-4923694 |
| chr1: 194583312-194583313 | chr17: 80726019-80726020 | chr5: 8974289-8974290 |
| chr1: 200826104-200826105 | chr17: 82085473-82085474 | chr5: 9014700-9014701 |
| chr1: 203029690-203029691 | chr17: 82117862-82117863 | chr5: 17731641-17731642 |
| chr1: 207710761-207710762 | chr17: 82121044-82121045 | chr5: 43373115-43373116 |
| chr1: 207715415-207715416 | chr17: 82121081-82121082 | chr5: 55864175-55864176 |
| chr1: 207716022-207716023 | chr17: 82128282-82128283 | chr5: 59406233-59406234 |
| chr1: 207728860-207728861 | chr17: 82149856-82149857 | chr5: 67606414-67606415 |
| chr1: 208380840-208380841 | chr17: 82152874-82152875 | chr5: 67669656-67669657 |
| chr1: 210433024-210433025 | chr17: 82153081-82153082 | chr5: 73129834-73129835 |
| chr1: 210438875-210438876 | chr17: 82154757-82154758 | chr5: 73159595-73159596 |
| chr1: 218669424-218669425 | chr17: 82157570-82157571 | chr5: 76701012-76701013 |
| chr1: 221269232-221269233 | chr17: 82158926-82158927 | chr5: 78023707-78023708 |
| chr1: 222581923-222581924 | chr17: 82159344-82159345 | chr5: 84785906-84785907 |
| chr1: 225705699-225705700 | chr17: 82178542-82178543 | chr5: 93844837-93844838 |
| chr1: 225706797-225706798 | chr17: 82179297-82179298 | chr5: 99358997-99358998 |
| chr1: 229252064-229252065 | chr17: 82190470-82190471 | chr5: 115985426-115985427 |
| chr1: 229264900-229264901 | chr17: 82193225-82193226 | chr5: 118207894-118207895 |
| chr1: 229334434-229334435 | chr17: 82196808-82196809 | chr5: 121633993-121633994 |
| chr1: 230996850-230996851 | chr17: 82199920-82199921 | chr5: 149710929-149710930 |
| chr1: 231601954-231601955 | chr17: 82204400-82204401 | chr5: 172560034-172560035 |
| chr1: 234258268-234258269 | chr17: 82204505-82204506 | chr5: 173459301-173459302 |
| chr1: 247090597-247090598 | chr18: 3781414-3781415 | chr5: 173528180-173528181 |
| chr10: 2387625-2387626 | chr18: 6943264-6943265 | chr5: 173654386-173654387 |
| chr10: 7587240-7587241 | chr18: 7393790-7393791 | chr5: 178322641-178322642 |
| chr10: 11791282-11791283 | chr18: 7408415-7408416 | chr5: 180431369-180431370 |
| chr10: 11792647-11792648 | chr18: 10280328-10280329 | chr5: 180437175-180437176 |
| chr10: 14779734-14779735 | chr18: 13566625-13566626 | chr5: 180437206-180437207 |
| chr10: 15304449-15304450 | chr18: 28226714-28226715 | chr6: 866372-866373 |
| chr10: 15715629-15715630 | chr18: 31286317-31286318 | chr6: 934777-934778 |
| chr10: 36676311-36676312 | chr18: 48776605-48776606 | chr6: 1030922-1030923 |
| chr10: 36717685-36717686 | chr18: 54166542-54166543 | chr6: 1035009-1035010 |
| chr10: 43115110-43115111 | chr18: 59486892-59486893 | chr6: 8068015-8068016 |
| chr10: 43177327-43177328 | chr18: 62899736-62899737 | chr6: 10149072-10149073 |
| chr10: 43180273-43180274 | chr18: 63407154-63407155 | chr6: 11618884-11618885 |
| chr10: 43251994-43251995 | chr18: 73749757-73749758 | chr6: 17157980-17157981 |
| chr10: 43253841-43253842 | chr18: 75187119-75187120 | chr6: 20644262-20644263 |
| chr10: 47997139-47997140 | chr18: 77148499-77148500 | chr6: 25515898-25515899 |
| chr10: 67758163-67758164 | chr18: 78913287-78913288 | chr6: 26085789-26085790 |
| chr10: 67896279-67896280 | chr18: 78915875-78915876 | chr6: 36400391-36400392 |
| chr10: 67896570-67896571 | chr19: 6701501-6701502 | chr6: 36620363-36620364 |
| chr10: 67902149-67902150 | chr19: 6732970-6732971 | chr6: 59463587-59463588 |
| chr10: 71382697-71382698 | chr19: 10245308-10245309 | chr6: 68865910-68865911 |
| chr10: 71389058-71389059 | chr19: 16302584-16302585 | chr6: 70190919-70190920 |
| chr10: 71741104-71741105 | chr19: 18059573-18059574 | chr6: 71996180-71996181 |
| chr10: 74306802-74306803 | chr19: 21056349-21056350 | chr6: 72006991-72006992 |
| chr10: 76383179-76383180 | chr19: 22839022-22839023 | chr6: 72013022-72013023 |
| chr10: 76699065-76699066 | chr19: 23326569-23326570 | chr6: 72230810-72230811 |
| chr10: 76789353-76789354 | chr19: 23484796-23484797 | chr6: 72311799-72311800 |
| chr10: 77298787-77298788 | chr19: 23568016-23568017 | chr6: 72345568-72345569 |
| chr10: 77347376-77347377 | chr19: 28779881-28779882 | chr6: 74215097-74215098 |
| chr10: 78062439-78062440 | chr19: 28817223-28817224 | chr6: 74218797-74218798 |
| chr10: 78738299-78738300 | chr19: 39135160-39135161 | chr6: 74219671-74219672 |
| chr10: 80450884-80450885 | chr19: 44367127-44367128 | chr6: 74243294-74243295 |
| chr10: 86255416-86255417 | chr19: 45352885-45352886 | chr6: 74243922-74243923 |
| chr10: 95619469-95619470 | chr19: 45364779-45364780 | chr6: 74263040-74263041 |
| chr10: 95630438-95630439 | chr19: 47269511-47269512 | chr6: 75581622-75581623 |
| chr10: 95632508-95632509 | chr19: 49468339-49468340 | chr6: 83486335-83486336 |
| chr10: 95752208-95752209 | chr19: 50351979-50351980 | chr6: 86332656-86332657 |
| chr10: 96643789-96643790 | chr19: 53188889-53188890 | chr6: 99535308-99535309 |
| chr10: 103819548-103819549 | chr19: 53200369-53200370 | chr6: 102401461-102401462 |
| chr10: 104510648-104510649 | chr19: 53207345-53207346 | chr6: 107192690-107192691 |
| chr10: 117779783-117779784 | chr19: 53210432-53210433 | chr6: 109371281-109371282 |
| chr10: 120945393-120945394 | chr2: 4883406-4883407 | chr6: 109399146-109399147 |
| chr10: 120960870-120960871 | chr2: 4884289-4884290 | chr6: 122199313-122199314 |
| chr10: 122983069-122983070 | chr2: 4925507-4925508 | chr6: 122234096-122234097 |
| chr10: 124012802-124012803 | chr2: 8178513-8178514 | chr6: 127492318-127492319 |
| chr10: 127595478-127595479 | chr2: 8236098-8236099 | chr6: 129735373-129735374 |
| chr10: 129202497-129202498 | chr2: 8241775-8241776 | chr6: 132216266-132216267 |
| chr10: 130027881-130027882 | chr2: 14200165-14200166 | chr6: 138923549-138923550 |
| chr11: 386177-386178 | chr2: 22669765-22669766 | chr6: 141484432-141484433 |
| chr11: 2328525-2328526 | chr2: 22744154-22744155 | chr6: 141685202-141685203 |
| chr11: 2563672-2563673 | chr2: 28995030-28995031 | chr6: 150945580-150945581 |
| chr11: 8313666-8313667 | chr2: 31202019-31202020 | chr6: 153405270-153405271 |
| chr11: 11282937-11282938 | chr2: 36058984-36058985 | chr6: 155229871-155229872 |
| chr11: 15187845-15187846 | chr2: 38079793-38079794 | chr6: 155236186-155236187 |
| chr11: 15693660-15693661 | chr2: 47729542-47729543 | chr6: 155260770-155260771 |
| chr11: 15696125-15696126 | chr2: 54323871-54323872 | chr6: 155269950-155269951 |
| chr11: 15717313-15717314 | chr2: 54326971-54326972 | chr6: 155292983-155292984 |
| chr11: 16958605-16958606 | chr2: 54341024-54341025 | chr6: 155296058-155296059 |
| chr11: 18024439-18024440 | chr2: 54345454-54345455 | chr6: 155303464-155303465 |
| chr11: 26144626-26144627 | chr2: 54350426-54350427 | chr6: 155306095-155306096 |
| chr11: 26590801-26590802 | chr2: 54356928-54356929 | chr6: 155429084-155429085 |
| chr11: 31905142-31905143 | chr2: 54433405-54433406 | chr6: 157890467-157890468 |
| chr11: 32823364-32823365 | chr2: 67600945-67600946 | chr6: 157917686-157917687 |
| chr11: 32853379-32853380 | chr2: 71124356-71124357 | chr6: 158577538-158577539 |
| chr11: 35502109-35502110 | chr2: 75712808-75712809 | chr6: 160713907-160713908 |
| chr11: 37555332-37555333 | chr2: 78006878-78006879 | chr6: 162466914-162466915 |
| chr11: 41830927-41830928 | chr2: 85416026-85416027 | chr6: 163938847-163938848 |
| chr11: 41877561-41877562 | chr2: 86074095-86074096 | chr6: 168628960-168628961 |
| chr11: 41882415-41882416 | chr2: 109316230-109316231 | chr7: 553383-553384 |
| chr11: 45815905-45815906 | chr2: 128403049-128403050 | chr7: 2538820-2538821 |
| chr11: 62327422-62327423 | chr2: 129460845-129460846 | chr7: 2551426-2551427 |
| chr11: 63116208-63116209 | chr2: 129561335-129561336 | chr7: 3036418-3036419 |
| chr11: 68255563-68255564 | chr2: 129786025-129786026 | chr7: 3208458-3208459 |
| chr11: 68257081-68257082 | chr2: 129822326-129822327 | chr7: 5393040-5393041 |
| chr11: 68302626-68302627 | chr2: 129835012-129835013 | chr7: 6477808-6477809 |
| chr11: 74556104-74556105 | chr2: 133440272-133440273 | chr7: 10735749-10735750 |
| chr11: 75515874-75515875 | chr2: 133723645-133723646 | chr7: 11249173-11249174 |
| chr11: 78892995-78892996 | chr2: 133761999-133762000 | chr7: 13171175-13171176 |
| chr11: 84508689-84508690 | chr2: 134480403-134480404 | chr7: 13183056-13183057 |
| chr11: 85086743-85086744 | chr2: 137108562-137108563 | chr7: 15389613-15389614 |
| chr11: 86736623-86736624 | chr2: 138267413-138267414 | chr7: 31815954-31815955 |
| chr11: 86736700-86736701 | chr2: 138274270-138274271 | chr7: 32029916-32029917 |
| chr11: 93509677-93509678 | chr2: 145665075-145665076 | chr7: 32082687-32082688 |
| chr11: 99531263-99531264 | chr2: 150629135-150629136 | chr7: 36266627-36266628 |
| chr11: 101363528-101363529 | chr2: 152971636-152971637 | chr7: 37221181-37221182 |
| chr11: 101387512-101387513 | chr2: 153690409-153690410 | chr7: 41710488-41710489 |
| chr11: 101423242-101423243 | chr2: 159368072-159368073 | chr7: 51400114-51400115 |
| chr11: 101440627-101440628 | chr2: 159371622-159371623 | chr7: 52817696-52817697 |
| chr11: 115689465-115689466 | chr2: 169302477-169302478 | chr7: 67889567-67889568 |
| chr11: 116892317-116892318 | chr2: 171572218-171572219 | chr7: 78874428-78874429 |
| chr11: 121335779-121335780 | chr2: 171596578-171596579 | chr7: 80101245-80101246 |
| chr11: 123226161-123226162 | chr2: 171596992-171596993 | chr7: 80323438-80323439 |
| chr11: 123226952-123226953 | chr2: 171599550-171599551 | chr7: 81979081-81979082 |
| chr11: 124972887-124972888 | chr2: 171605560-171605561 | chr7: 84024267-84024268 |
| chr11: 125923793-125923794 | chr2: 214307181-214307182 | chr7: 88883726-88883727 |
| chr11: 126412001-126412002 | chr2: 223389403-223389404 | chr7: 91926042-91926043 |
| chr11: 127465734-127465735 | chr2: 223823348-223823349 | chr7: 101948757-101948758 |
| chr11: 127560360-127560361 | chr2: 223827178-223827179 | chr7: 106830546-106830547 |
| chr12: 269605-269606 | chr2: 230910128-230910129 | chr7: 106876690-106876691 |
| chr12: 8257246-8257247 | chr2: 237468368-237468369 | chr7: 122898233-122898234 |
| chr12: 9875529-9875530 | chr2: 237517240-237517241 | chr7: 129579198-129579199 |
| chr12: 14227148-14227149 | chr2: 237523647-237523648 | chr7: 129663261-129663262 |
| chr12: 16818992-16818993 | chr2: 238827723-238827724 | chr7: 129795796-129795797 |
| chr12: 19957659-19957660 | chr2: 238841897-238841898 | chr7: 130302526-130302527 |
| chr12: 25246038-25246039 | chr2: 240169036-240169037 | chr7: 130337894-130337895 |
| chr12: 25345225-25345226 | chr2: 240538169-240538170 | chr7: 134633964-134633965 |
| chr12: 25826890-25826891 | chr2: 240538178-240538179 | chr7: 139066869-139066870 |
| chr12: 27813113-27813114 | chr20: 10783358-10783359 | chr7: 143491643-143491644 |
| chr12: 29524007-29524008 | chr20: 15947360-15947361 | chr7: 145144093-145144094 |
| chr12: 29539679-29539680 | chr20: 18904363-18904364 | chr7: 145144304-145144305 |
| chr12: 29539738-29539739 | chr20: 18916250-18916251 | chr7: 145147385-145147386 |
| chr12: 31009680-31009681 | chr20: 21628230-21628231 | chr7: 146640291-146640292 |
| chr12: 32294550-32294551 | chr20: 22766420-22766421 | chr7: 150659009-150659010 |
| chr12: 32295299-32295300 | chr20: 22774356-22774357 | chr7: 153447310-153447311 |
| chr12: 32316592-32316593 | chr20: 22774694-22774695 | chr8: 3693797-3693798 |
| chr12: 33858934-33858935 | chr20: 23053918-23053919 | chr8: 6012061-6012062 |
| chr12: 45696597-45696598 | chr20: 23093210-23093211 | chr8: 10817989-10817990 |
| chr12: 46214834-46214835 | chr20: 23384456-23384457 | chr8: 15628888-15628889 |
| chr12: 46220858-46220859 | chr20: 24744094-24744095 | chr8: 16650817-16650818 |
| chr12: 46283677-46283678 | chr20: 24764537-24764538 | chr8: 20315924-20315925 |
| chr12: 46333986-46333987 | chr20: 50704241-50704242 | chr8: 20364394-20364395 |
| chr12: 46436992-46436993 | chr20: 51615760-51615761 | chr8: 29090387-29090388 |
| chr12: 52215339-52215340 | chr20: 51621376-51621377 | chr8: 31159020-31159021 |
| chr12: 52224265-52224266 | chr20: 51625175-51625176 | chr8: 38671268-38671269 |
| chr12: 73525936-73525937 | chr20: 51649503-51649504 | chr8: 40691773-40691774 |
| chr12: 77390688-77390689 | chr20: 51655315-51655316 | chr8: 40979073-40979074 |
| chr12: 77405104-77405105 | chr20: 51694428-51694429 | chr8: 41772886-41772887 |
| chr12: 86014547-86014548 | chr20: 53668291-53668292 | chr8: 49453825-49453826 |
| chr12: 96688660-96688661 | chr20: 53877808-53877809 | chr8: 55164230-55164231 |
| chr12: 96775182-96775183 | chr20: 57464821-57464822 | chr8: 63107273-63107274 |
| chr12: 96841834-96841835 | chr20: 61663369-61663370 | chr8: 70176495-70176496 |
| chr12: 96901126-96901127 | chr20: 61665867-61665868 | chr8: 72971368-72971369 |
| chr12: 98429242-98429243 | chr20: 62896919-62896920 | chr8: 81163165-81163166 |
| chr12: 102712923-102712924 | chr20: 62934159-62934160 | chr8: 89034067-89034068 |
| chr12: 105711044-105711045 | chr21: 19392721-19392722 | chr8: 95870285-95870286 |
| chr12: 120768690-120768691 | chr21: 26103786-26103787 | chr8: 96534294-96534295 |
| chr12: 123499393-123499394 | chr21: 26735659-26735660 | chr8: 101671885-101671886 |
| chr12: 129147634-129147635 | chr21: 32054987-32054988 | chr8: 103112224-103112225 |
| chr12: 130464545-130464546 | chr21: 40353698-40353699 | chr8: 105607963-105607964 |
| chr12: 130736803-130736804 | chr21: 42136782-42136783 | chr8: 111321594-111321595 |
| chr13: 18503669-18503670 | chr21: 42137625-42137626 | chr8: 111415482-111415483 |
| chr13: 21224306-21224307 | chr21: 46003474-46003475 | chr8: 113099988-113099989 |
| chr13: 29621913-29621914 | chr21: 46003722-46003723 | chr8: 113655159-113655160 |
| chr13: 29695343-29695344 | chr22: 20167774-20167775 | chr8: 115923585-115923586 |
| chr13: 29697391-29697392 | chr22: 26408249-26408250 | chr8: 118652242-118652243 |
| chr13: 29714764-29714765 | chr22: 35574506-35574507 | chr8: 120365541-120365542 |
| chr13: 29783165-29783166 | chr22: 39908577-39908578 | chr8: 128045716-128045717 |
| chr13: 29842500-29842501 | chr22: 39910189-39910190 | chr8: 128096450-128096451 |
| chr13: 42560688-42560689 | chr22: 42628404-42628405 | chr8: 129419413-129419414 |
| chr13: 43134602-43134603 | chr22: 44172197-44172198 | chr8: 130596461-130596462 |
| chr13: 45542244-45542245 | chr22: 45611743-45611744 | chr8: 134338064-134338065 |
| chr13: 49412660-49412661 | chr22: 46486568-46486569 | chr8: 144840067-144840068 |
| chr13: 50751057-50751058 | chr22: 46486735-46486736 | chr8: 144840646-144840647 |
| chr13: 50756289-50756290 | chr22: 49666536-49666537 | chr8: 144847374-144847375 |
| chr13: 62100286-62100287 | chr3: 3439293-3439294 | chr9: 7711546-7711547 |
| chr13: 66062024-66062025 | chr3: 3455554-3455555 | chr9: 7744667-7744668 |
| chr13: 66062246-66062247 | chr3: 6412886-6412887 | chr9: 7790321-7790322 |
| chr13: 66231576-66231577 | chr3: 9451631-9451632 | chr9: 16996036-16996037 |
| chr13: 66244073-66244074 | chr3: 9506275-9506276 | chr9: 20569814-20569815 |
| chr13: 69258311-69258312 | chr3: 9516928-9516929 | chr9: 25102077-25102078 |
| chr13: 69393052-69393053 | chr3: 11620845-11620846 | chr9: 27287366-27287367 |
| chr13: 80991136-80991137 | chr3: 27793807-27793808 | chr9: 28171824-28171825 |
| chr13: 84326823-84326824 | chr3: 29688518-29688519 | chr9: 28985199-28985200 |
| chr13: 88970486-88970487 | chr3: 30188551-30188552 | chr9: 33324244-33324245 |
| chr13: 89250772-89250773 | chr3: 40916200-40916201 | chr9: 35656878-35656879 |
| chr13: 98803100-98803101 | chr3: 41549751-41549752 | chr9: 38390940-38390941 |
| chr13: 99877721-99877722 | chr3: 42793712-42793713 | chr9: 71281515-71281516 |
| chr13: 100952437-100952438 | chr3: 49983003-49983004 | chr9: 83329689-83329690 |
| chr13: 101168575-101168576 | chr3: 51101415-51101416 | chr9: 87399628-87399629 |
| chr13: 104116360-104116361 | chr3: 51415308-51415309 | chr9: 95888739-95888740 |
| chr13: 104117227-104117228 | chr3: 55760366-55760367 | chr9: 99800585-99800586 |
| chr13: 107071933-107071934 | chr3: 57605640-57605641 | chr9: 99809014-99809015 |
| chr13: 107091003-107091004 | chr3: 58572328-58572329 | chr9: 101744825-101744826 |
| chr13: 107115270-107115271 | chr3: 73654989-73654990 | chr9: 103186774-103186775 |
| chr13: 107126142-107126143 | chr3: 74111274-74111275 | chr9: 106728119-106728120 |
| chr14: 30313957-30313958 | chr3: 75869717-75869718 | chr9: 107715477-107715478 |
| chr14: 30777652-30777653 | chr3: 80713420-80713421 | chr9: 107716609-107716610 |
| chr14: 30796613-30796614 | chr3: 103740541-103740542 | chr9: 112093972-112093973 |
| chr14: 38103975-38103976 | chr3: 103756204-103756205 | chr9: 112110432-112110433 |
| chr14: 38104321-38104322 | chr3: 107351515-107351516 | chr9: 114150865-114150866 |
| chr14: 38106703-38106704 | chr3: 114174495-114174496 | chr9: 115147014-115147015 |
| chr14: 38107126-38107127 | chr3: 117675063-117675064 | chr9: 115147063-115147064 |
| chr14: 39790496-39790497 | chr3: 135481931-135481932 | chr9: 128808785-128808786 |
| chr14: 39925237-39925238 | chr3: 140650686-140650687 | chr9: 130377517-130377518 |
| chr14: 41505096-41505097 | chr3: 149109309-149109310 | chr9: 130385105-130385106 |
| chr14: 52368906-52368907 | chr3: 153329695-153329696 | chr9: 134711335-134711336 |
| chr14: 52737402-52737403 | chr3: 157331535-157331536 | chr9: 136640786-136640787 |
| chr14: 52747973-52747974 | chr3: 159905770-159905771 | chrX: 5473613-5473614 |
| chr14: 100772361-100772362 | chr3: 169209665-169209666 | chrX: 13404029-13404030 |
| chr14: 100774192-100774193 | chr3: 176606667-176606668 | chrX: 23565826-23565827 |
| chr14: 104254216-104254217 | chr3: 177712960-177712961 | chrX: 74349641-74349642 |
| chr14: 104269891-104269892 | chr3: 178950326-178950327 | chrX: 74381544-74381545 |
| chr15: 22654506-22654507 | chr3: 181353477-181353478 | chrX: 97762509-97762510 |
| chr15: 26002303-26002304 | chr3: 183194494-183194495 | chrX: 97782571-97782572 |
| chr15: 26433461-26433462 | chr3: 183209474-183209475 | chrX: 117093087-117093088 |
| chr15: 26599095-26599096 | chr3: 183828336-183828337 | chrX: 119743876-119743877 |
| chr15: 26622138-26622139 | chr3: 187600439-187600440 | chrX: 127584044-127584045 |
| chr15: 26746855-26746856 | chr3: 187918377-187918378 | chrX: 129679718-129679719 |
| chr15: 30950995-30950996 | chr3: 187965257-187965258 | chrX: 136773142-136773143 |
| chr15: 36329345-36329346 | chr3: 187978939-187978940 | chrX: 147737736-147737737 |
| chr15: 45236942-45236943 | chr3: 189163604-189163605 | chrX: 151725896-151725897 |
| chr15: 50312619-50312620 | chr3: 189182694-189182695 | |
| chr15: 50348387-50348388 | chr3: 190035048-190035049 | |

TABLE 2
CpG methylation sites correlated
with AD and their rank

| Rank | CpG site |
|---|---|
| 1 | chr19: 49468339-49468340 |
| 2 | chr13: 29697391-29697392 |
| 3 | chr2: 171599550-171599551 |
| 4 | chr2: 223823348-223823349 |
| 5 | chr2: 38079793-38079794 |
| 6 | chr2: 223389403-223389404 |
| 7 | chr18: 7393790-7393791 |
| 8 | chr1: 169668153-169668154 |
| 9 | chr13: 29714764-29714765 |
| 10 | chr2: 54341024-54341025 |
| 11 | chr2: 54323871-54323872 |
| 12 | chr2: 223827178-223827179 |
| 13 | chr3: 107351515-107351516 |
| 14 | chr1: 218669424-218669425 |
| 15 | chr4: 112701336-112701337 |
| 16 | chr16: 85241293-85241294 |
| 17 | chr2: 78006878-78006879 |
| 18 | chr13: 66231576-66231577 |
| 19 | chr10: 77298787-77298788 |
| 20 | chr9: 114150865-114150866 |
| 21 | chr10: 77347376-77347377 |
| 22 | chr6: 155229871-155229872 |
| 23 | chr3: 177712960-177712961 |
| 24 | chr6: 74219671-74219672 |
| 25 | chr2: 129786025-129786026 |
| 26 | chr16: 1257295-1257296 |
| 27 | chr6: 155303464-155303465 |
| 28 | chr14: 30313957-30313958 |
| 29 | chr15: 58474236-58474237 |
| 30 | chr6: 99535308-99535309 |
| 31 | chr5: 73159595-73159596 |
| 32 | chr17: 32964779-32964780 |
| 33 | chr9: 28171824-28171825 |
| 34 | chr13: 100952437-100952438 |
| 35 | chr6: 74243294-74243295 |
| 36 | chr1: 68275770-68275771 |
| 37 | chr5: 172560034-172560035 |
| 38 | chr20: 62896919-62896920 |
| 39 | chr7: 106830546-106830547 |
| 40 | chr11: 126412001-126412002 |
| 41 | chr6: 155296058-155296059 |
| 42 | chr2: 67600945-67600946 |
| 43 | chr12: 19957659-19957660 |
| 44 | chr18: 10280328-10280329 |
| 45 | chr17: 78274971-78274972 |
| 46 | chr13: 66062024-66062025 |
| 47 | chr10: 43251994-43251995 |
| 48 | chr7: 41710488-41710489 |
| 49 | chr6: 74263040-74263041 |
| 50 | chr13: 21224306-21224307 |
| 51 | chr11: 15187845-15187846 |
| 52 | chr7: 143491643-143491644 |
| 53 | chr2: 54356928-54356929 |
| 54 | chr13: 66244073-66244074 |
| 55 | chr4: 21511949-21511950 |
| 56 | chr1: 79919480-79919481 |
| 57 | chr8: 55164230-55164231 |
| 58 | chr1: 194583312-194583313 |
| 59 | chr9: 136640786-136640787 |
| 60 | chr3: 30188551-30188552 |
| 61 | chr17: 82193225-82193226 |
| 62 | chr10: 76383179-76383180 |
| 63 | chr6: 11618884-11618885 |
| 64 | chr1: 221269232-221269233 |
| 65 | chr16: 11519017-11519018 |
| 66 | chr10: 47997139-47997140 |
| 67 | chr2: 134480403-134480404 |
| 68 | chr3: 9451631-9451632 |
| 69 | chr13: 42560688-42560689 |
| 70 | chr9: 71281515-71281516 |
| 71 | chr10: 67758163-67758164 |
| 72 | chr3: 192505947-192505948 |
| 73 | chr17: 76392900-76392901 |
| 74 | chr7: 80101245-80101246 |
| 75 | chr5: 180431369-180431370 |
| 76 | chr12: 46436992-46436993 |
| 77 | chr1: 168588906-168588907 |
| 78 | chr9: 103186774-103186775 |
| 79 | chr8: 72971368-72971369 |
| 80 | chr20: 23053918-23053919 |
| 81 | chrX: 74381544-74381545 |
| 82 | chr1: 103529898-103529899 |
| 83 | chr3: 42793712-42793713 |
| 84 | chr14: 39925237-39925238 |
| 85 | chr15: 100504533-100504534 |
| 86 | chr18: 59486892-59486893 |
| 87 | chr6: 25515898-25515899 |
| 88 | chr11: 125923793-125923794 |
| 89 | chr6: 74218797-74218798 |
| 90 | chr17: 82204400-82204401 |
| 91 | chr11: 101423242-101423243 |
| 92 | chr6: 155429084-155429085 |
| 93 | chr7: 3036418-3036419 |
| 94 | chr13: 66062246-66062247 |
| 95 | chrX: 97762509-97762510 |
| 96 | chr11: 121335779-121335780 |
| 97 | chr1: 207728860-207728861 |
| 98 | chr5: 78023707-78023708 |
| 99 | chr6: 36400391-36400392 |
| 100 | chr8: 31159020-31159021 |
| 101 | chr1: 95836889-95836890 |
| 102 | chr4: 101185199-101185200 |
| 103 | chr6: 10149072-10149073 |
| 104 | chr17: 66695786-66695787 |
| 105 | chr4: 187572796-187572797 |
| 106 | chr16: 87353981-87353982 |
| 107 | chr11: 101387512-101387513 |
| 108 | chr9: 38390940-38390941 |
| 109 | chr4: 172879114-172879115 |
| 110 | chr7: 36266627-36266628 |
| 111 | chr4: 138306908-138306909 |
| 112 | chr10: 130027881-130027882 |
| 113 | chr17: 82152874-82152875 |
| 114 | chr7: 32029916-32029917 |
| 115 | chr6: 74215097-74215098 |
| 116 | chr4: 101171837-101171838 |
| 117 | chr8: 81163165-81163166 |
| 118 | chr19: 21056349-21056350 |
| 119 | chr17: 82199920-82199921 |
| 120 | chrX: 136773142-136773143 |
| 121 | chr7: 51400114-51400115 |
| 122 | chr1: 229252064-229252065 |
| 123 | chr4: 4284517-4284518 |
| 124 | chr3: 190046403-190046404 |
| 125 | chr6: 155292983-155292984 |
| 126 | chr2: 129822326-129822327 |
| 127 | chr7: 553383-553384 |
| 128 | chr21: 42136782-42136783 |
| 129 | chr1: 203029690-203029691 |
| 130 | chr22: 42628404-42628405 |
| 131 | chr2: 237468368-237468369 |
| 132 | chr11: 123226952-123226953 |
| 133 | chr6: 86332656-86332657 |
| 134 | chr14: 52737402-52737403 |
| 135 | chr17: 82196808-82196809 |
| 136 | chr7: 81979081-81979082 |
| 137 | chr1: 207715415-207715416 |
| 138 | chr20: 61663369-61663370 |
| 139 | chr8: 128045716-128045717 |
| 140 | chr1: 26572634-26572635 |
| 141 | chr15: 84743786-84743787 |
| 142 | chr1: 79768860-79768861 |
| 143 | chr13: 45542244-45542245 |
| 144 | chr1: 210438875-210438876 |
| 145 | chr6: 162466914-162466915 |
| 146 | chr3: 3455554-3455555 |
| 147 | chr6: 107192690-107192691 |
| 148 | chr7: 130302526-130302527 |
| 149 | chr4: 4281948-4281949 |
| 150 | chr5: 173459301-173459302 |
| 151 | chr2: 237523647-237523648 |
| 152 | chr2: 133761999-133762000 |
| 153 | chr6: 59463587-59463588 |
| 154 | chr2: 159371622-159371623 |
| 155 | chr11: 26144626-26144627 |
| 156 | chr6: 129735373-129735374 |
| 157 | chrX: 74349641-74349642 |
| 158 | chr9: 20569814-20569815 |
| 159 | chr2: 171596578-171596579 |
| 160 | chr17: 11335771-11335772 |
| 161 | chr20: 22774694-22774695 |
| 162 | chr21: 46003722-46003723 |
| 163 | chr11: 116892317-116892318 |
| 164 | chr10: 95632508-95632509 |
| 165 | chr4: 172859547-172859548 |
| 166 | chr16: 25785755-25785756 |
| 167 | chr17: 82117862-82117863 |
| 168 | chr18: 78915875-78915876 |
| 169 | chr19: 39135160-39135161 |
| 170 | chr12: 129147634-129147635 |
| 171 | chr17: 78293563-78293564 |
| 172 | chr10: 43180273-43180274 |
| 173 | chr7: 6477808-6477809 |
| 174 | chr4: 88076723-88076724 |
| 175 | chr1: 171891978-171891979 |
| 176 | chr2: 171572218-171572219 |
| 177 | chr9: 112110432-112110433 |
| 178 | chr14: 100774192-100774193 |
| 179 | chr10: 120945393-120945394 |
| 180 | chr1: 169022201-169022202 |
| 181 | chr12: 25345225-25345226 |
| 182 | chr18: 6943264-6943265 |
| 183 | chr2: 47729542-47729543 |
| 184 | chr6: 150945580-150945581 |
| 185 | chr1: 188259784-188259785 |
| 186 | chr17: 82204505-82204506 |
| 187 | chr18: 13566625-13566626 |
| 188 | chr2: 28995030-28995031 |
| 189 | chr15: 50312619-50312620 |
| 190 | chr3: 135481931-135481932 |
| 191 | chr6: 141484432-141484433 |
| 192 | chr13: 107115270-107115271 |
| 193 | chr1: 163782298-163782299 |
| 194 | chr11: 15693660-15693661 |
| 195 | chr20: 18904363-18904364 |
| 196 | chr1: 25000294-25000295 |
| 197 | chr5: 17731641-17731642 |
| 198 | chr17: 82159344-82159345 |
| 199 | chr11: 99531263-99531264 |
| 200 | chr15: 69236063-69236064 |
| 20 | chr15: 86862172-86862173 |
| 202 | chr4: 61426568-61426569 |
| 203 | chr18: 62899736-62899737 |
| 204 | chr15: 67901611-67901612 |
| 205 | chr13: 107126142-107126143 |
| 206 | chr2: 85416026-85416027 |
| 207 | chr3: 9506275-9506276 |
| 208 | chr8: 16650817-16650818 |
| 209 | chr19: 23484796-23484797 |
| 210 | chr2: 75712808-75712809 |
| 211 | chr1: 119183000-119183001 |
| 212 | chr5: 84785906-84785907 |
| 213 | chr20: 18916250-18916251 |
| 214 | chr6: 157890467-157890468 |
| 215 | chr17: 82121081-82121082 |
| 216 | chr12: 46333986-46333987 |
| 217 | chr18: 78913287-78913288 |
| 218 | chr17: 5956533-5956534 |
| 219 | chr3: 51415308-51415309 |
| 220 | chr9: 7711546-7711547 |
| 221 | chr19: 45352885-45352886 |
| 222 | chr16: 84853958-84853959 |
| 223 | chr10: 43115110-43115111 |
| 224 | chr17: 82178542-82178543 |
| 225 | chr9: 33324244-33324245 |
| 226 | chr1: 112039744-112039745 |
| 227 | chr19: 22839022-22839023 |
| 228 | chr11: 123226161-123226162 |
| 229 | chr17: 32958992-32958993 |
| 230 | chr16: 78049572-78049573 |
| 231 | chr2: 240538169-240538170 |
| 232 | chr20: 50704241-50704242 |
| 233 | chr21: 40353698-40353699 |
| 234 | chr3: 183194494-183194495 |
| 235 | chr12: 46214834-46214835 |
| 236 | chr15: 26433461-26433462 |
| 237 | chr2: 129460845-129460846 |
| 238 | chr19: 53188889-53188890 |
| 239 | chr9: 107716609-107716610 |
| 240 | chr19: 28779881-28779882 |
| 241 | chr8: 134338064-134338065 |
| 242 | chr5: 43373115-43373116 |
| 243 | chr2: 153690409-153690410 |
| 244 | chr11: 41882415-41882416 |
| 245 | chr12: 29539738-29539739 |
| 246 | chr14: 30777652-30777653 |
| 247 | chr11: 18024439-18024440 |
| 248 | chr11: 86736700-86736701 |
| 249 | chr20: 51615760-51615761 |
| 250 | chr20: 24764537-24764538 |
| 251 | chr3: 149109309-149109310 |
| 252 | chr1: 26303390-26303391 |
| 253 | chr7: 32082687-32082688 |
| 254 | chr4: 26052795-26052796 |
| 255 | chr20: 51655315-51655316 |
| 256 | chr22: 46486568-46486569 |
| 257 | chr4: 3759619-3759620 |
| 258 | chr21: 32054987-32054988 |
| 259 | chr7: 129579198-129579199 |
| 260 | chr13: 62100286-62100287 |
| 261 | chr6: 155260770-155260771 |
| 262 | chrX: 129679718-129679719 |
| 263 | chr17: 82179297-82179298 |
| 264 | chr4: 84622305-84622306 |
| 265 | chr12: 25246038-25246039 |
| 266 | chr16: 12572421-12572422 |
| 267 | chr7: 13183056-13183057 |
| 268 | chr20: 53668291-53668292 |
| 269 | chr2: 238827723-238827724 |
| 270 | chr4: 88216166-88216167 |
| 271 | chr16: 17928367-17928368 |
| 272 | chr7: 2551426-2551427 |
| 273 | chr15: 22654506-22654507 |
| 274 | chr2: 109316230-109316231 |
| 275 | chr6: 122199313-122199314 |
| 276 | chr2: 71124356-71124357 |
| 277 | chr6: 17157980-17157981 |
| 278 | chr9: 107715477-107715478 |
| 279 | chr2: 31202019-31202020 |
| 280 | chr2: 171605560-171605561 |
| 281 | chr10: 71389058-71389059 |
| 282 | chr16: 56970976-56970977 |
| 283 | chr2: 129835012-129835013 |
| 284 | chr10: 2387625-2387626 |
| 285 | chr17: 82153081-82153082 |
| 286 | chr2: 145665075-145665076 |
| 287 | chr17: 82157570-82157571 |
| 288 | chr22: 39908577-39908578 |
| 289 | chr2: 150629135-150629136 |
| 290 | chr4: 140129688-140129689 |
| 291 | chr4: 188852114-188852115 |
| 292 | chr12: 46220858-46220859 |
| 293 | chr19: 53207345-53207346 |
| 294 | chr1: 147723335-147723336 |
| 295 | chr5: 173654386-173654387 |
| 296 | chr1: 147032287-147032288 |
| 297 | chr7: 134633964-134633965 |
| 298 | chr12: 120768690-120768691 |
| 299 | chr11: 32853379-32853380 |
| 300 | chr3: 140650686-140650687 |

TABLE 3
Twenty chromosomal positions of
methylation sites correlated with AD

| | |
|---|---|
| chr3: 107351515-107351516 | chr1: 169668153-169668154 |
| chr9: 114150865-114150866 | chr10: 77298787-77298788 |
| chr1: 218669424-218669425 | chr18: 7393790-7393791 |
| chr16: 85241293-85241294 | chr2: 78006878-78006879 |
| chr19: 49468339-49468340 | chr2: 171599550-171599551 |
| chr2: 38079793-38079794 | chr13: 29714764-29714765 |
| chr2: 223827178-223827179 | chr13: 29697391-29697392 |
| chr2: 223823348-223823349 | chr13: 66231576-66231577 |
| chr4: 112701336-112701337 | chr2: 54341024-54341025 |
| chr2: 223389403-223389404 | chr2: 54323871-54323872 |

### EXAMPLES

The following examples are provided to illustrate particular features of certain aspects of the disclosure, but the scope of the claims should not be limited to those features exemplified.

Studying human age-dependent disorders is a long-standing challenge, especially for inaccessible tissues like the human brain. Sporadic late-onset Alzheimer's disease (LOAD) accounts for 95% of all AD cases (Querfurth et al., N. Engl. J. Med. 362, 329-344, 2010). Unlike the early-onset familial AD that is linked to genetic mutations in a specific gene, such as those found in APP, PSEN1 and PSEN2 genes, LOAD is thought to be caused by a complex combination of multiple genes and environmental factors, largely aligning to several age-related co-morbidities. Elucidating the complex genetic background interactions and epigenetic regulation that likely contribute to LOAD is critical to developing targeted therapies (Sen et al., Cell 166, 822-839, 2016). DNA methylation, the most studied epigenetic system in mammals, has been confirmed to play a crucial role in multiple human diseases such as cancer, imprinting and repeat-instability disorders (Robertson et al., Nat. Rev. Genet. 6, 597-310, 2005). Intriguingly, aberrant DNA methylation is observed in normal aging processes, highlighting the link between proper epigenetic regulation and age-dependent cellular functions.

To characterize genome-wide LOAD-specific methylation signatures from in vivo brain cell types, aligning previous work with current brain cell type atlas efforts led by BRAIN Initiative Cell Census Network (BICCN) (Ecker et al., Neuron 96, 542-557, 2022; BRAIN Initiative Cell Census Network, Nature 598, 86-102, 2021; Tian et al., bioRxiv 2022.11.30.518285. 10.1101/2022.11.30.518285, 2022), single-nucleus methyl-3C sequencing (sn-m3C-seq) was performed to jointly profile chromatin conformation and methylome from the same cell (Lee et al., Nat. Methods 16, 999-1006; Luo et al., Cell Genom 2.10.1016/j.xgen.2022.100107, 2022). This approach enabled the definition of the cell type taxonomy in AD patients and identified differentially methylated regions between AD and control (aDMRs) within and across brain cell types and revealing erosion of the epigenome in single brain cells of LOAD patients based on cell type-specific 3D genome structure alterations. These findings in human AD patients are consistent with the observations on loss of epigenetic information in aged mice (Yang et al., Cell 186, 305-326.e27, 2023) and more recently in an AD mouse model and human aged cerebellar granule neurons (Dileep et al., Cell 186, 4404-4421.e20, 2023; Tan et al., Science 381, 1112-1119).

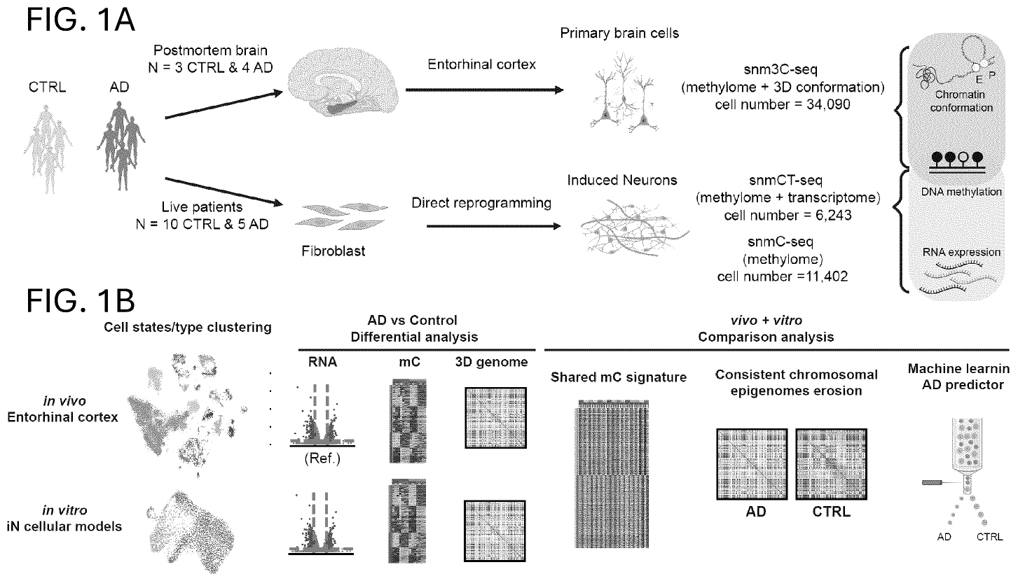

In addition, to assess whether the epigenetic signatures found from in vivo human brain tissues can be detected in cellular models, induced neurons (iNs) were directly converted from dermal fibroblasts of LOAD patients and generated snmCT-seq datasets capturing transcriptome and methylome of fibroblasts and iNs. The distinct cell states of in vitro cellular iN models was defined and characterized epigenetic signatures of AD from age-retaining iNs. A comparative analysis between in vitro cellular models and in vivo primary brain tissues identified conserved and robust methylation signatures. A reliable set of machine learning model selected CpG sites showed very high accuracy of AD prediction across in vitro and in vivo cell types. In summary, a comprehensive dataset dissecting the underlying molecular alterations involved in epigenetic regulation and 3D chromatin conformations of in vivo primary brain tissues and in vitro cellular iN models was generated (FIGS. 1A-1B).

### Example 1

### Materials and Methods

### Ethics Statement and IRB Clinical Information of AHA-Allen Cohort

Based on the clinical criteria published by the Consortium to Establish a Registry for Alzheimer's Disease (CERAD), National Institutes of Health (NIH) standards, and Braak staging, subjects in AHA-Allen cohorts were recruited by Shiley-Marcos UCSD Alzheimer's Disease Center. Dermal human fibroblasts and postmortem entorhinal cortex were collected with informed consent and strict adherence to legal and ethical guidelines from patients of the Shiley-Marcos UCSD Alzheimer's Disease Center.

Human iPSC Lines and Generation of iNs

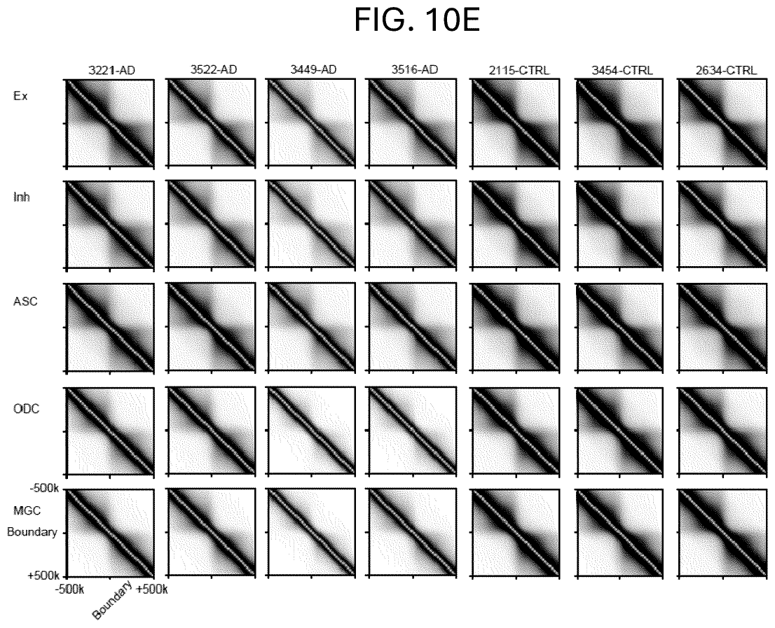

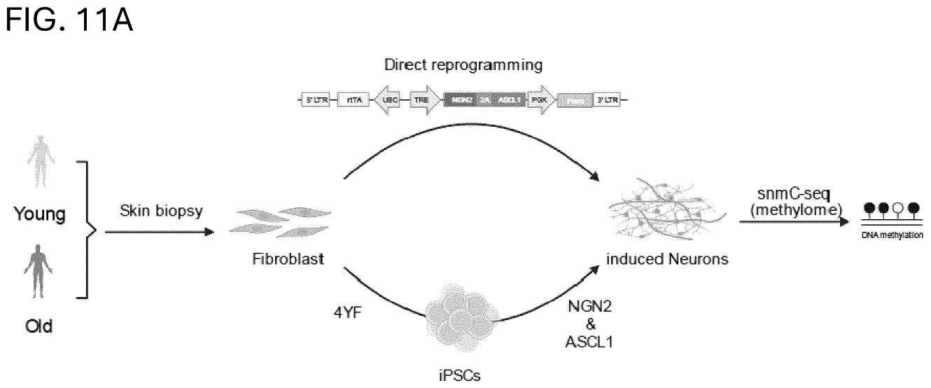

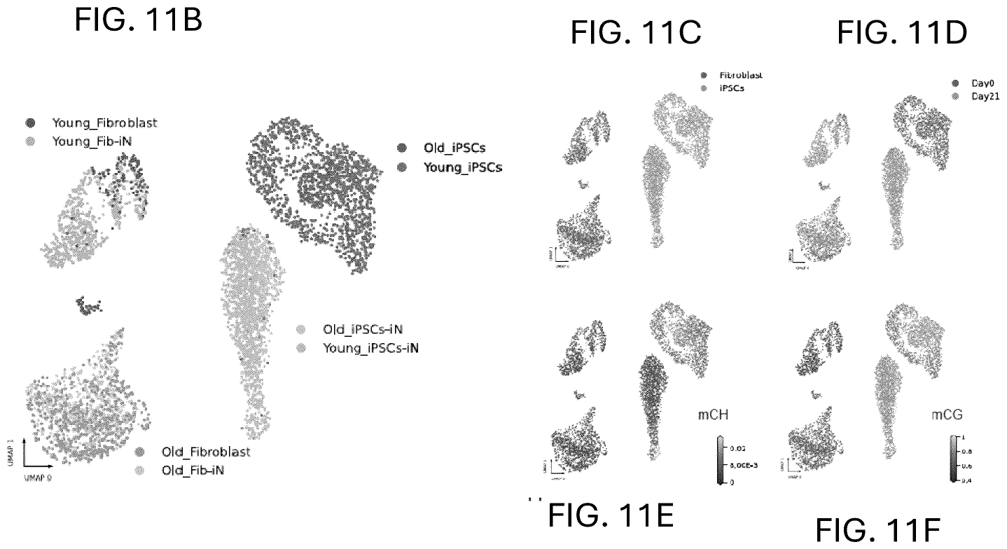

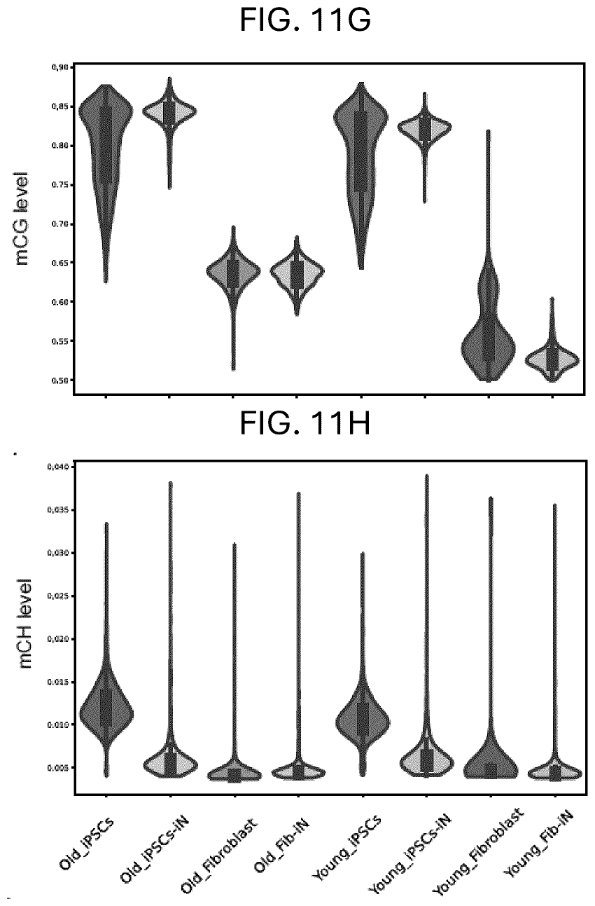

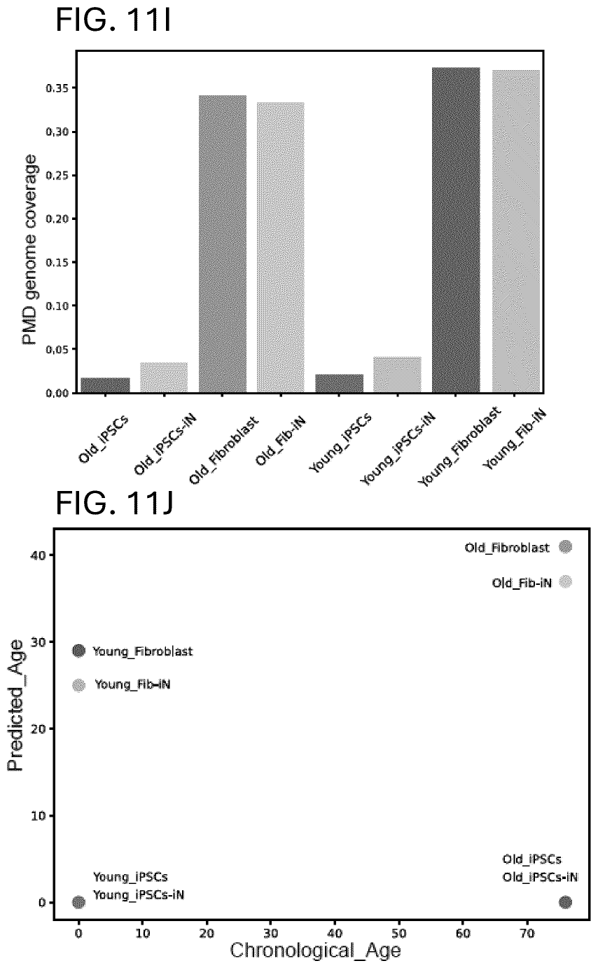

Human iPSCs were obtained from the Salk Stem Cell Core. Fibroblasts were reprogrammed via CytoTune™-iPS 2.0 Sendai Reprogramming Kit (ThermoFisher Cat #A165167) per manufacturer recommendations. All iPSCs were karyotypically validated via g-banded karyotyping (WiCell) and were regularly screened for mycoplasma via MycoAlert™ PLUS Mycoplasma Detection Kit (Lonza Cat #75860-362). Two major approaches to generating neurons in a dish are based on overexpression of proneural factors combined with chemicals from iPSCs differentiation or directly converted from fibroblasts. Age-dependent transcriptional signatures are more likely to be retained in directly converted neurons from fibroblasts rather than in differentiated iPSCs (Mertens et al., Anuu. Rev. Genet. 52, 2018). To assess different differentiation strategies to generate iNs and characterize their epigenome modality, a single nucleus methylome sequencing (snmC-seq) was conducted to profile the methylome of iN cells differentiated from either human pluripotent stem cells or human fibroblast cells via the overexpression of proneuronal factor NGN2 approach. Using young (1 yr. old, male) and aged (76 yrs. old, male) fibroblasts as cell resources, it was found that iNs generated from fibroblasts retained aging methylation features and individual differentially methylated region (DMR) signatures, whereas the iPSC-iN method did not. DMRs were erased during iPSC reprogramming and reconfigured during NPC differentiation, and iPSC-iN cells from young and aged samples became indistinguishable (FIGS. 11A-11J). Direct conversion of iNs was performed via doxycycline-inducible NGN2-P2A-ASCL1 (N2A) as previously described (Mertens et al., Anuu. Rev. Genet. 52, 2018). Briefly, stable N2A fibroblast lines were generated with lentivirus. Fibroblasts were maintained in dense cultures and passaged three times under puromycin selection before induction. Upon confluence, media was replaced with neural conversion media containing doxycycline for 21 days. For single-nuclei experiments, iNs were washed with PBS, incubated for 20 minutes at 37° C. with TrypLE (Gibco cat #12604039), diluted in PBS up to 15 mL, pelleted at 100×g for 5 minutes, aspirated, and snap frozen.

Nuclei Purification from iN Vivo Fibroblasts and iNs Cells for snmCT-Seq

Cultured fibroblast and induced iN cells in the dish were dissociated in TrpLE medium. Cells were counted and aliquoted at 1 million per experimental sample and then pelleted by centrifugation at 100×g for 5 min. The supernatant medium was aspirated, and cell pellets were resuspended in 600 μl NIBT [250 mM Sucrose, 10 mM Tris-Cl pH=8, 25 mM KCl, 5 mM MgCl2, 0.1% Triton X-100, 1 mM DTT, 1:100 Proteinase inhibitor (Sigma-Aldrich P830), 1:1000 SUPERaseIn RNase Inhibitor (ThermoFisher Scientific AM2694), and 1:1000 RNaseOUT RNase Inhibitor (ThermoFisher Scientific 10777019). After gently pipetting up and down 40 times, the lysate was mixed with 400 ml of 50% Iodixanol (Sigma-Aldrich D1556) and loaded on top of a 500 ml 25% Iodixanol cushion. Nuclei were pelleted by centrifugation at 10,000×g at 4° C. for 20 minutes using a swing rotor. The pellet was resuspended in 2 mL of DPBS supplemented with 1:1000 SUPERaseIn RNase Inhibitor and 1:1000 RNaseOUT RNase Inhibitor. Hoechst 33342 was added to the sample to a final concentration of 1.25 nM and incubated on ice for 5 minutes for nuclei staining. Nuclei were pelleted by 1,000×g at 4° C. for 10 minutes and resuspended in 1 mL of DPBS supplemented with RNase inhibitors.

snmCT-Seq Library Preparation

The optimized snmCT-seq library preparation is based on the snmCAT-seq published previously (Luo et al., Cell Genom 2.10.1016/j.xgen.2022.100107, 2022). A detailed bench protocol can be found online at protocols.io/view/snmcat-v2-x54v9jbylg3e/v2. In general, the purified nuclei were sorted into a 384-well plate (ThermoFisher 4483285) containing 1 μl mCT reverse transcription reaction per well. The mCT reverse transcription reaction contained 1× Superscript II First-Strand Buffer, 5 mM DTT, 0.1% Triton X-100, 2.5 mM MgCl2, 30 mM NaCl, 500 mM each of 50-methyl-dCTP (NEB N0356S), dATP, dTTP and dGTP, 1.2 mM dT30VN_5 oligo-dT primer, 2.4 mM TSO_4 template switching oligo, 2 mM N6_3 random primer, 1 U RNaseOUT RNase inhibitor, 0.5 U SUPERaseIn RNase inhibitor, and 10 U Superscript II Reverse Transcriptase (ThermoFisher 18064-071). The plates were placed in a thermocycler and incubated using the following program: 25° C. for 5 minutes, 42° C. for 90 minutes, 10 cycles of 50° C. for 2 minutes and 42° C. for 2 minutes, 85° C. 5 minutes followed by 4° C. Three μl of cDNA amplification mix was added into each snmCT-seq reverse transcription reaction. Each cDNA amplification reaction contained 1×KAPA 2G Buffer A, 600 nM ISPCR23_3 PCR primer, and 0.08 U KAPA2G Robust HotStart DNA Polymerase (5 U/mL, Roche KK5517). PCR reactions were performed using a thermocycler with the following conditions: 95° C. 3 minutes->[95° C. 15 seconds->60° C. 30 seconds->72° C. 2 minutes]->72° C. 5 minutes->4° C. The cycling steps were repeated for 12 cycles. One μl uracil cleavage mix was added into cDNA amplification reaction. Each 1 μl uracil cleavage mix contained 0.5 μl Uracil DNA Glycosylase (Enzymatics G5010) and 0.5 μl Elution Buffer (QIAGEN 19086). Unincorporated DNA oligos were digested at 37° C. for 30 minutes using a thermocycler. After addition of 25 μl of conversion reagent (Zymo Research) was added to each well of a 384-well plate, the following bisulfite conversion and library preparation was based on snmC-seq2 (described previously, Luo et al., BioRxiv 294355.10.1101/294355, 2018) and on an updated version snmC-seq3 used in BICCN (Liu et al., bioRxiv. 10.1101/2023.04.16.536509, 2023).

Nuclei Purification from Human Postmortem Tissues for snm3C-Seq

Brain blocks were ground in liquid nitrogen with cold mortar and pestle and then aliquoted and stored at −80° C. Approximately 100 mg of ground tissue was resuspended in 3 mL NIBT as above. The lysate was transferred to a pre-chilled 7 mL Dounce homogenizer (Sigma-Aldrich D9063) and Dounced using loose and tight pestles for 40 times each. The lysate was then mixed with 2 mL of 50% Iodixanol (Sigma-Aldrich D1556) to generate a nuclei suspension with 20% Iodixanol. One ml of the nuclei suspension was gently transferred on top of a 500 ml 25% Iodixanol cushion in each of the 5 freshly prepared 2-ml microcentrifuge tubes. Nuclei were pelleted by centrifugation at 10,000×g at 4° C. for 20 minutes using a swing rotor. The pellet was resuspended in 1 ml of DPBS supplemented with 1:1000 SUPERaseIn RNase Inhibitor and 1:1000 RNaseOUT RNase Inhibitor. A 10-μl aliquot of the suspension was taken for nuclei counting using a Biorad TC20 Automated Cell Counter. One million nuclei aliquots were pelleted by 1,000×g at 4° C. for 10 minutes and resuspended in 800 μl of ice-cold DPBS.

snm3C-Seq Library Preparation

The purified nuclei for snm3C-seq were cross-linked with additional digestion and ligation to capture in situ long-range DNA interaction following a modified protocol of Arima-3C kit (Arima Genomics). A detailed bench protocol can be found in the BICCN atlas paper (Liu et al., bioRxiv. 10.1101/2023.04.16.536509, 2023).

### Automation and Illumina Sequencing

The prepared nuclei from either snmCT-seq or snm3C-seq were sorted into a 384-well plate by Influx (BD) on a one-drop single mode. Then the automation handling of plates and library preparation for both snmCT-seq and snm3C-seq libraries followed the same bisulfite conversion-based methylation sequencing pipelines described previously (Luo et al., Science 357, 600-604, 2017; Luo et al., BioRxiv 294355.10.1101/294355, 2018) and an updated version snmC-seq3 used in BICCN (Liu et al., bioRxiv. 10.1101/2023.04.16.536509, 2023). To facilitate large-scale profiling, Beckman Biomek i7 instrument was used and running scripts were shared (Liu et al., bioRxiv. 10.1101/2023.04.16.536509, 2023). The snm3C-seq, snmCT-seq and snmC-seq libraries were sequenced on an Illumina Novae 6000 instrument using one S4 flow cell per 16 384-well plates on 150-bp paired-end mode.

### Quantification and Statistical Analysis

### Single-Cell Methylation and Multi-Omics Data Mapping (Alignment, Quality Control (QC))

The snmC-seq3, snmCT-seq and snm3C-seq mapping was calculated using the YAP pipeline (semba-data package, v1.6.8, hq-1.gitbook.io/mc/) as previously described (Luo et al. Cell Geno 2.10.1016/j.xgen.2022.100107, 2022; Liu et al., Nature 598, 120-128, 2021). The major steps of the processing steps include:
1) Demultiplexing FASTQ files into single cells (cutadapt (Martin et al., EMBnet.journal 17, 10-12, 2011), v2.10);
2) reads level QC;
   For snmCT-Seq (Methylome Part):
3a) Reads from step 2 were mapped onto human hg38 genome (one-pass mapping for snmCT-seq, two-pass mapping for snm3C) (bismark v0.20 (Krueger et al., Bioinformatics 27, 1571-1572, 2011), bowtie2 v2.3 (Langmead et al., Nat. Methods 9, 357-359, 2012));
4a) PCR duplicates were removed using Picard MarkDuplicates, the non-redundant reads were filtered by MAPQ >10. To select genomic reads from the filtered BAM, the “XM-tag” was generated by Bismark to calculate reads methylation level and keep reads with mCH ratio <0.5 and the number of cytosines ≥3.
5a) Tab-delimited (ALLC) files containing methylation level for every cytosine position were generated using allcools (Liu et al., BioRxiv. 10.1101/2023.04.16.536509, 2023) (v1.0.8) bam-to-allc function on the BAM file from step 4a.
   For snmCT-Seq (RNA Part):
3b) To map transcriptome reads, reads from step 2 were mapped to GENCODE human v30 indexed hg38 genome using STAR (v2.7.3a; Dobin et al., Bioinformatics 29, 15-21, 2013) with the following parameters: --alignEndsType Local --outSAMstrandField intronMotif --outSAMtype BAM Unsorted --outSAMunmapped None --outSAMattributes NH HI AS NM MD --sjdbOverhang 100 --outFilterType BySJout --outFilterMultimapNmax 20 --alignSJoverhangMin 8 --alignSJDBoverhangMin 1 --outFilterMismatchNmax 999′ #ENCODE standard options --outFilterMismatchNoverLmax 0.04 --alignIntronMin 20 --alignIntronMax 1000000 --alignMatesGapMax 1000000 --outFileNamePrefix rna_bam/TotalRNA
4b) The STAR mapped reads were first filtered by MAPQ >10. To select RNA reads from the filtered BAM, the “MD” tag was used to calculate reads methylation level and kept reads with mCH ratio >0.9 and the number of cytosines ≥3. The stringency of read partitioning was tested previously (Luo et al., Cell Genom 2.10.1016/j.xgen.2022.100107, 2022).
5b) BAM files from step 4b were counted across gene annotations using featureCount (1.6.4; Liao et al., Bioinformatics 30, 923-930, 2014) with the default parameters. Gene expression was quantified using either only exonic reads with “-t exon” or both exonic and intronic reads with “-t gene.”
   For snm3C-Seq (3C Modality Part):
4b) After the initial mC reads alignment as above, unmapped reads were retained and split into 3 pieces by 40 bp, 42 bp, and 40 bp resulting in six subreads (read1 and read2). The subreads derived from unmapped reads were mapped separately using HISAT-3N (Zhang et al., Genome Res. 10.1101/gr.275193.120, 2021) adapted in YAP pipeline (cemba-data package). All aligned reads were merged into BAM using Picard SortSam tool with query names sorted. For each fragment, the outermost aligned reads were chosen for the chromatin conformation map generation. The chromatin contacts and following analysis were processed using the scHiCluster described previously (Zhou et al., PNAS 116, 14011-14018, 2019) (online at zhoujt1994.github.io/scHiCluster/intro.html).
   Preprocessing of snmC-Seq, snmCT-Seq and snm3C-Seq Data

Primary QC for DNA methylome cells was (1) overall mCCC level <0.05; (2) overall mCH level <0.2; (3) overall mCG level <0.5; (4) total final DNA reads >100,000 and <10,000,000; and (5) Bismarck mapping rate >0.5. Note that the mCCC level estimates the upper bound of the cell-level bisulfite non-conversion rate. Additionally, lambda DNA spike-in methylation levels was calculated to estimate each sample's non-conversion rate. For the transcriptome modality in snmCT-seq, only the cells containing <5% mitochondrial reads, total RNA reads >5,000 were kept. For snm3C-seq cells, cis-long-range (two anchors >2500 bp apart) >50,000 was also required.

Clustering Analysis of snmCT-Seq and snm3C-Seq Data

For snmCT-seq (RNA part): The whole gene RNA read count matrix was used for snmCT-seq transcriptome analysis. Cells were filtered by the number of genes expressed >1,000 and genes were filtered by the number of cells expressed >10. The count matrix X was then normalized per cell and transformed by ln(X+1). After log transformation, the scanpy.pp.highly_variable_genes was used to select the top genes based on normalized dispersion. The selected feature matrix was scaled to unit variance and zero mean per feature followed by PCA calculation. To correct batch effects across individuals, a highly efficient framework based on the Seurat R integration algorithm was established (Hao et al., Cell 184, 3573-3587.e29, 2021). The integration framework consisted of 3 major steps to align snmCT-seq datasets on fibroblasts and iNs from different donors onto the same space: (1) using dimension reduction to derive embedding of the multiple datasets separated by donors in the same space; (2) using canonical correlation analysis (CCA) to capture the shared variance across cells between datasets and find anchors as 5 mutual nearest neighbors (MNN) between each two paired datasets; and (3) aligning the low-dimensional representation of the paired data sets together with the anchors.

To consensus clustering based on fixed resolution parameters (range from 0.2 to 0.6), Leiden clustering (Tragg et al., Sci. Rep. 9, 5233, 2019) was performed 200 times, using different random seeds. These result labels were then combined to establish preliminary cluster labels. Following this, predictive models were trained in the principal component (PC) space to predict labels and compute the confusion matrix. Finally, clusters with high similarity were merged to minimize confusion. The cluster selection was guided by the R1 and R2 normalization applied to the confusion matrix, as outlined in the SCCAF package (Miao et al., Nat. Methods 17, 621-628, 2020). This framework was incorporated in “ALLCools.clustering.ConsensusClustering” function.

For snmCT-seq (methylome part): The clustering analysis was performed with the mCH and mCG fractions of chrom100 k matrices described previously (Luo et al., Cell Genom 2.10.1016/j.xgen.2022.100107, 2022). Most functions were derived from allcools (Liu et al., BioRxiv. 10.1101/2023.04.16.536509, 2023), scanpy (Wolf et al., Genome Biol. 19, 15, 2018) and scikit-learn packages (Pedregosa et al., arXiv[sc.LG], 2825-2830, 2012). In general, the major steps in the clustering included: (1) feature filtering based on coverage, exclude ENCODE blacklist and located in autosomes; (2) Highly Variable Feature (HVF) selection; (3) generation of posterior chrom100 k mCH and mCG fraction matrices; (4) clustering with HVF and calculating Cluster Enriched Features (CEF) of the HVF clusters with “ALLCools.clustering.cluster_enriched_features” function; (5) calculating PC in the selected cell-by-CEF matrices and generating the t-SNE (Gmail and Hinton, jmlr.org/papers/volume9/vandermaatenO8a/vandermaatenO8a.pdf?fbcl, 2018) and UMAP (McInnes et al., arXiv [stat.ML], 2018) embeddings for visualization; and (6) consensus clustering process using “ALLCools.clustering.ConsensusClustering” function.

Identification of DEGs, DMRs and aDMRs Enriched Hotspots

After finalizing clustering in in vitro fibroblast-iN snmCT-seq data analysis, the paired strategy was used to calculate RNA DEGs within a specific cluster for AD-specific (AD versus CTRL) or within a specific individual line for conversional DEGs (fibroblast versus iNs). All the protein-coding and long non-coding RNA genes from hg38 gencode v30 were used with the scanpy.tl.rank_genes_group function with the Wilcoxon test and filtered the resulting marker gene by adjusted P value <0.01 and log 2 (fold-change) >1.

For DMRs identification, the single-cell ALLC files were merged into pseudo-bulk level using the “allcools merge-allc” command. Next, DMR calling was performed with methylpy (Schultz et al., Nature 523, 212-216, 2015) on a grouped pseudo-bulk allc files. For example, to identify AD-specific methylation signatures in fibroblasts and iN clusters, the samples from all individuals in AD and CTRL groups were merged separately and then DMRs were called between these two groups. After getting the primary set of DMRs, the methylation level at these DMRs was counted from all individuals using the “methylpy add-methylation-level” function. Additional filtering on the DMRs was performed by comparing the methylation levels among different individuals within groups using Student's t-test. Only DMRs with a minimum p-value less than 0.05 between any two groups were retained. The same processes were used to identify aDMRs in each specific brain cell type of snm3C-seq datasets. aDMRs enriched hotspots of the in vivo entorhinal cortex were identified by a sliding window of 5 kb bin across the autosomes, with normalized GC content. PyComplexHeatmap (Ding et al., Imeta. 10.1002/imt2.115, 2023) was used to visualize methylation level at these DMRs in the complex heatmaps.

Hypomethylated DMRs in the corresponding sample groups and cell types were labeled for better visualization. The heatmap rows were split according to sample groups, and the columns were split based on DMR groups and cell types. Within each subgroup, rows and columns were clustered using ward linkage and the Jaccard metric. The aDMRs-enriched hotspots were visualized by tagore package (Rishishwar et al., Sci. Rep. 5, 12376, 2015).

### Gene Set Enrichment Test, Motif Enrichment, Chromatin States and Functional Enrichment of DMRs

To validate the DEGs found in snmCT-seq dataset in vitro fibroblast/iN models, GO enrichment test was performed using GSEApy (Fang et al., Bioinformatic 39.10.1093/bioinformatics/btac757, 2023) and Enrichr (Kuleshov et al., Nucleic Acids Res. 44, W90-W97, 2016) open source. The -log(adjusted P value) of KEGG pathway enrichment in each selected gene set was color-coded on the enrichr combined score with KEGG terms. For motif enrichment analysis, the hypomethylated and hypermethylated DMRs reported by methylpy from the columns ‘hypermethylated_samples’ and ‘hypomethylated_samples’ was obtained. HOMER was used to identify enriched motifs within these different sets of DMRs for each comparison. The results from HOMER's ‘knownResults.txt’ output files were used for downstream analysis. Only motif enrichments with a p-value <0.01 were retained. The motif enrichment results were visualized using scatterplots in seaborn. To perform functional enrichment analysis of DMRs, GREAT (http://great.stanford.edu/public/html/index.php) was utilized. The genome feature annotation of aDMRs enriched hotspots and ML identified DMSs in the entorhinal cortex was conducted using “annotatePeaks.pl” functions in HOMER. The chromHMM states enrichment analysis of aDMRs were quantified by “bedtools intersect” the overlapping of aDMRs with the corresponding ChromHMM states based on histone ChiP-Seq peaks from the Roadmap Epigenomics project derived from frontal cortex (67 and 80 years old female donors), the accession number is ENCSR867UKF in the ENCODE database. Enrichment tests were performed using Fisher's tests with the significance of FDR adjusted p-value calculated by multiple tests.

Integration and Annotation Between snm3C-Seq Datasets and Human Brain Atlas

To integrate the snm3C-seq dataset to the reference human brain methylation atlas (HBA) (Tian et al., bioRxiv 2022.11.30.518285. 10.1101/2022.11.30.518285, 2022), methylation information from both CHN and CGN sites was used. Log scaled cell-by-100 kb-bin methylation fraction matrices were derived for CGN and CHN separately. After removing all low quality bins (hg38 genome blacklist, coverage<500, or coverage>3000), features that were both highly variable and cluster enriched in HBA were selected for PCA. The first 100 PCs of mCG and mCH matrices were normalized by their standard deviations and then concatenated horizontally for integration. Canonical correlation analysis (CCA) was used to capture the shared variance across cells between datasets and then selected 5 mutual nearest neighbors (MNNs) as anchors between the datasets. Next, HBA was used as a reference dataset to pull the dataset into the same space. More details on the integration algorithms are described in Tian et al (Tian et al., bioRxiv 2022.11.30.518285. 10.1101/2022.11.30.518285, 2022). Lastly, Harmony was used on the CCA integrated matrix for better integration between individuals. After integration, major cell types were annotated by the most numerous HBA cell type within each leiden cluster in the joint embedding.

Chromatin Contact Matrix and Preprocessing Imputation of snm3C-Seq Datasets Imputation was performed using scHiCluster (zhoujtl994.github.io/scHiCluster/intro.html) to the contact matrices at 100 kb, 25 kb and 10 kb resolution for single cell contacts within 10.05 Mb (100 kb and 25 kb), and 5.05 Mb (10 kb). For imputation at 10 kb resolution specifically, convolution and random walk were performed to speed up the imputation. For pseudo-bulk analysis, cells from each donor were merged by major cell type (ASC, MGC, ODC, Inh, Ex) with cell number across individuals as closely as possible to reduce bias created by different sequencing depth. Most cell types across individuals had at least 150 cells for pseudo-bulk analysis. For pseudo-bulk analysis that compared AD and CTRL cell types, the same number of cells (n=400) were randomly selected and merged among AD and CTRL individuals.

### Contacts, Loop, Domain, and Compartment Analysis

As described above, pseudo-bulk cell type groups were merged by individual and disease status. Imputed contact matrices were used for both single-cell and pseudo-bulk domain calling at 25 kb resolution and loop calling at 10 kb resolution. Raw contact matrices were used instead to infer A/B compartments for pseudo-bulk groups at 100 k resolution to better capture detailed genome interaction. Differential loops, domains, and compartments were derived as described previously (Tian et al., bioRxiv 2022.11.30.518285. 10.1101/2022.11.30.518285, 2022), so as saddle plots, compartment strengths, and loop summits. The cis (intra-chromosomal) contact probability normalized by CG counts for each cell was calculated. DNA contacts were binned by an exponent step of 0.125 with a base of 2, ranging from contact distance between 2500 bp to 249 Mb. The start and end of the bin were calculated by 2500×2\hat{ }0.125i and 2500×2\hat{ }0.125(i+1).

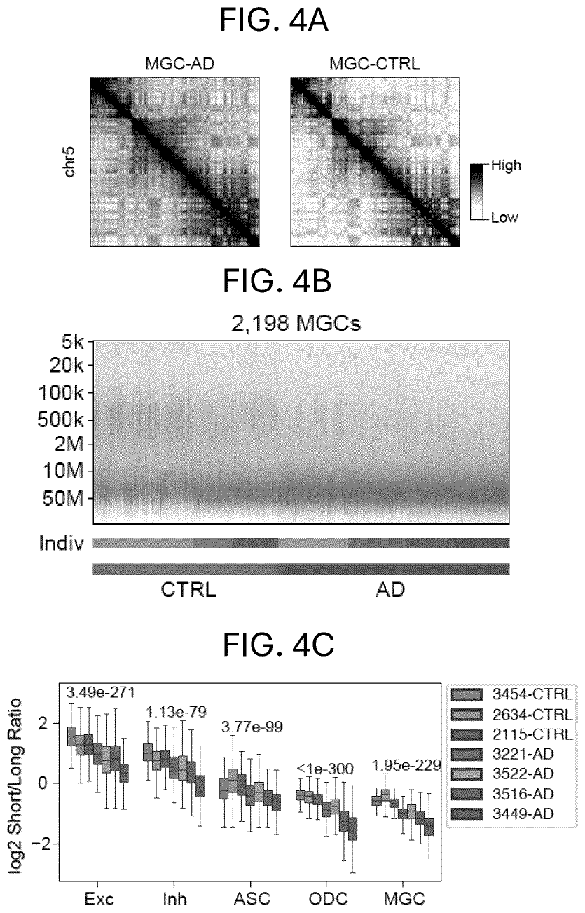

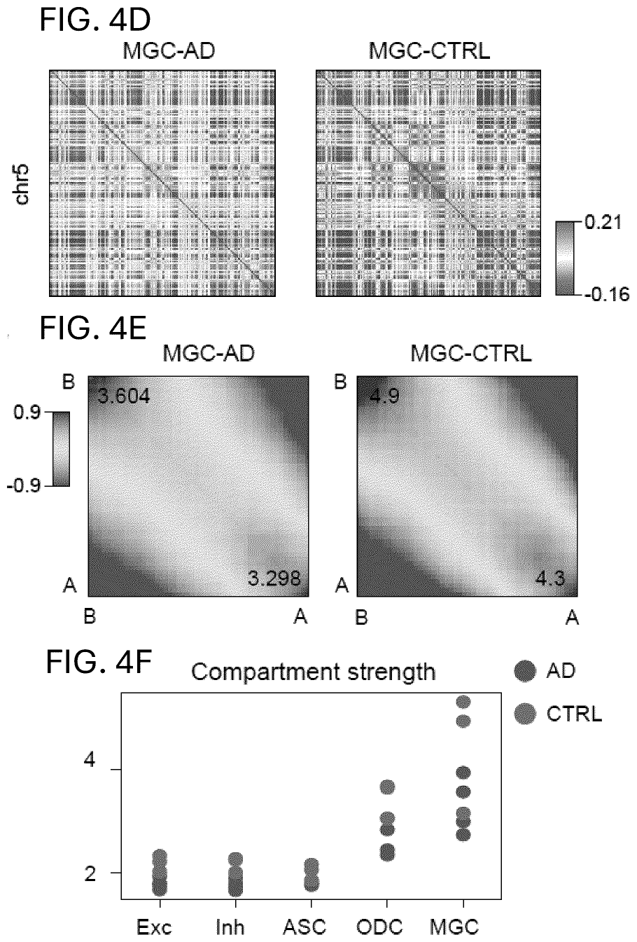

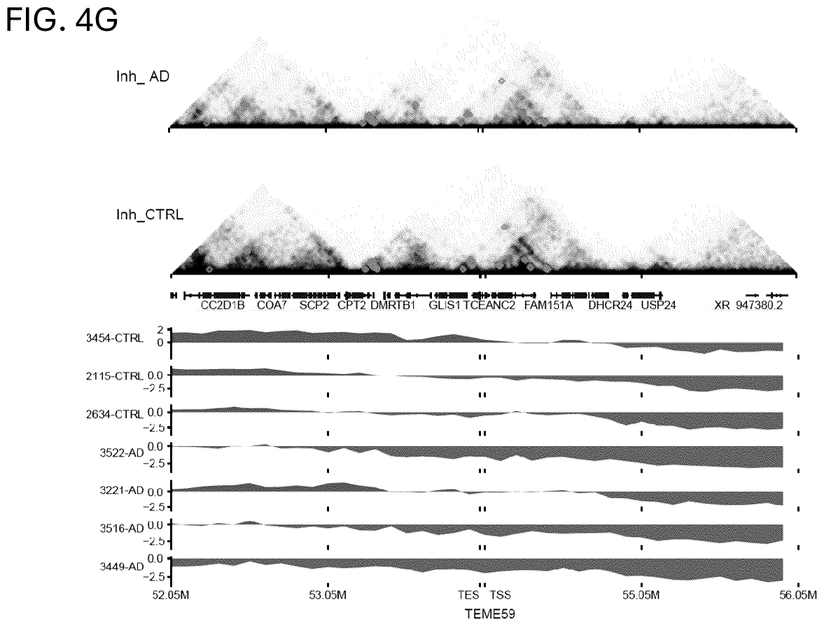

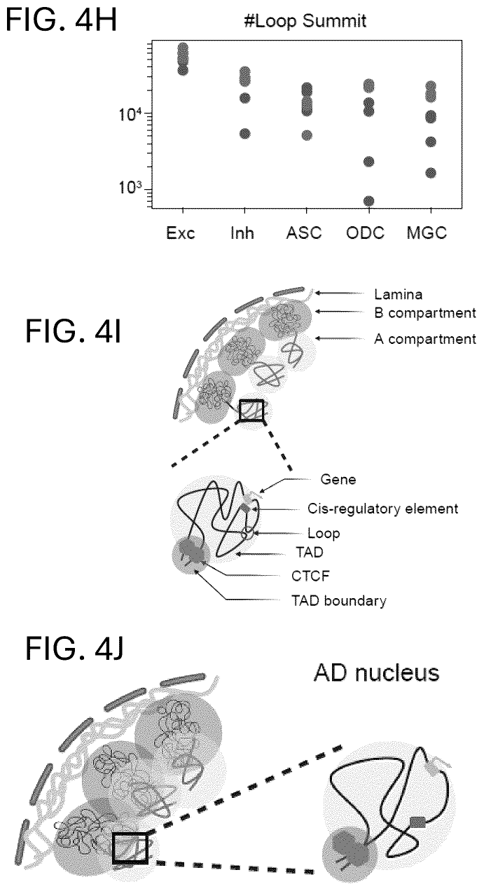

The short-long ratio in FIG. 4C was defined as the mean probability of contact in 51st (200 k) to 76th (2M) binds divided by the probability of contact in 103rd (20M) to 114th (50M) bins. Based on the loop interactions mapped in 3C contacts, aDMRs were assigned to a gene if the transcription start site (TSS) located in one 10 kb-bin had interactions with the bin where aDMRs were located. Then, aDRMs were considered as putative CREs of DEGs if the aDMRs paired with genes found differential expressed in published sn-RNA datasets between AD and CTRL (Morabito et al., Nature 53, 1143-1155, 2021)

### Determination of Reliable CpG Sites for AD Prediction

Identifying aDMRs to predict AD is multifaceted, involving various steps from preprocessing and feature selection to validation.

Data preprocessing: The initial step was to merge DMR sites between AD and CTRL groups for every cell type. The methylation fraction was then extracted for all these sites for every sample. To maintain data reliability, sites where the change in the methylation fraction across samples was less than 0.4 or the standard deviation was less than 0.1 were filtered out. Given the inherent biases in sample data, these data were further normalized within each sample using the z-score. Following this preprocessing, the resultant data served as the primary candidate set considered for subsequent feature selection.

Feature selection: An iterative feature selection approach was employed to ensure a comprehensive feature selection that captured as much reliable and informative data as possible. This was done over 30 rounds. Stratified 3-fold cross-validation (CV) was used in every round to train Random Forest classifiers (RFCs). The importance of the remaining features was gauged by the average feature importance derived from the RFCs. The top 500 features were chosen in every round, and the rest were reserved for the next round. The parameters set for the RFCs included utilizing 500 trees with a max_depth of 3 for each RFC.

Method evaluation: To ascertain the predictive capability of the selected features, a stratified 4-fold CV was performed, ensuring that the stratification was based on the combined label of AD versus CTRL and in vivo versus in vitro conditions. In each fold, the 3 training subsets underwent the feature selection process mentioned earlier. Following this, an RFC was trained based on the chosen features, which was then used on the remaining fold to determine its accuracy. After completing the 4-fold CV, the overall prediction accuracy was 97.1%.

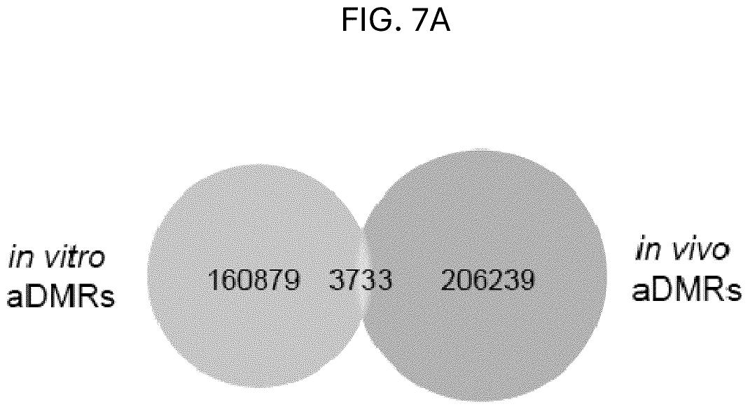

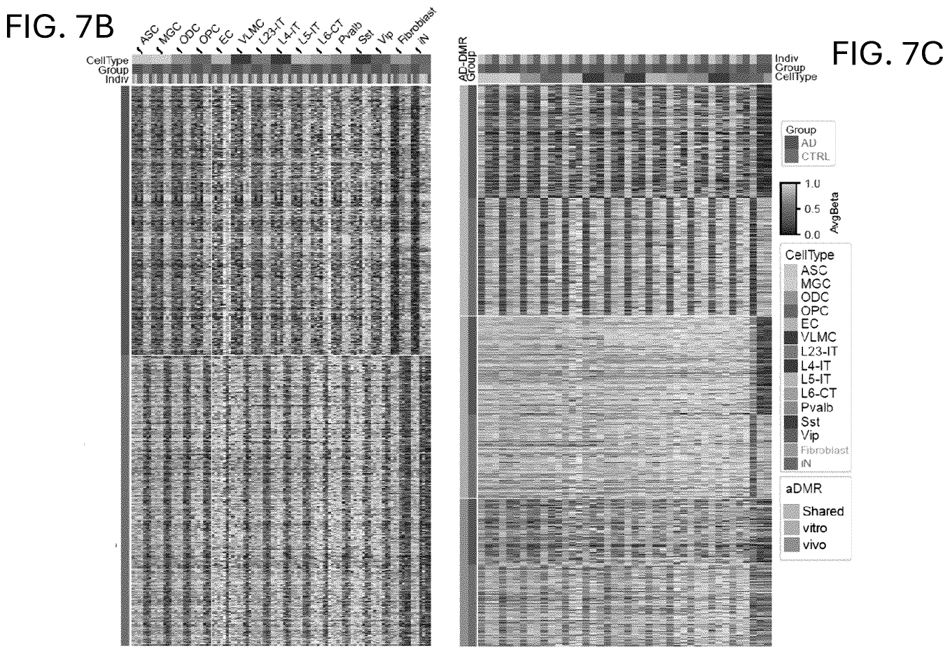

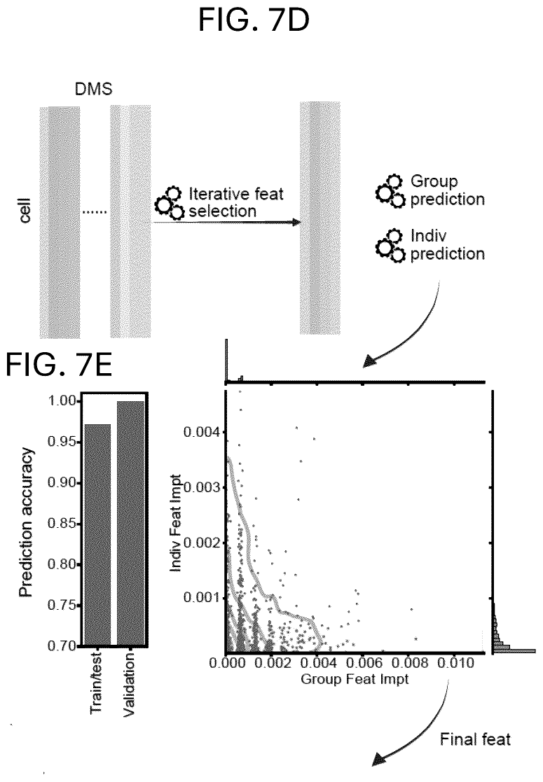

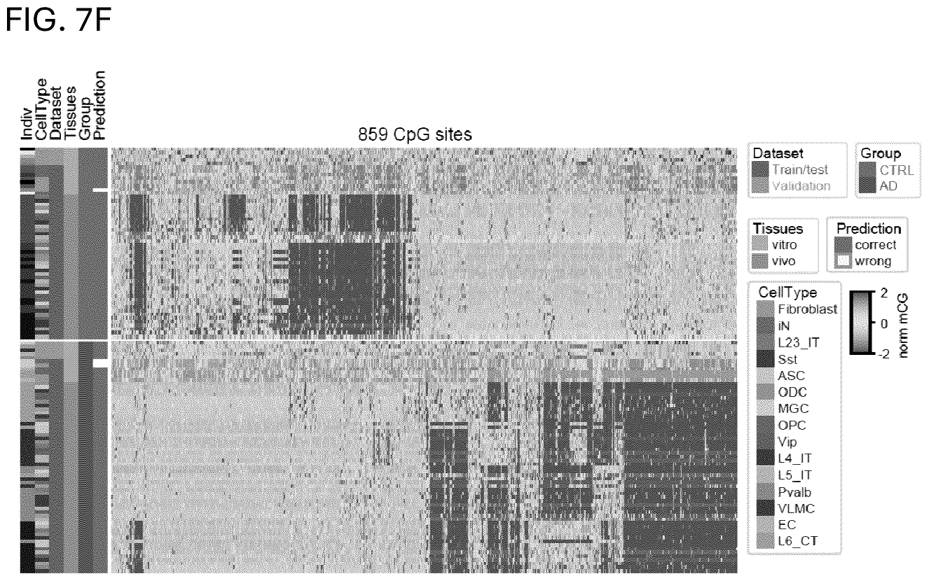

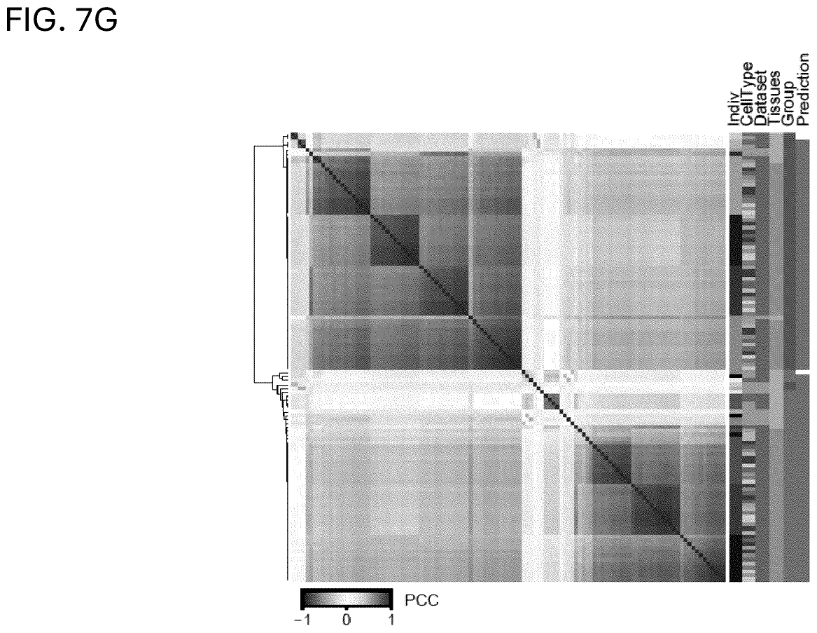

Mitigating donor effects: It is essential to account for donor variability. To do this, shared features from the prior 4-fold CV were selected as candidates. The importance in predicting AD vs. CTRL or determining the donor was then calculated. FIG. 7E demonstrates how each feature played a role in these 2 prediction tasks. Those features that held a positive importance for AD vs. CTRL predictions and an importance of less than 5e-4 for donor predictions were finalized. This meticulous process resulted in 859 CpG sites.

Final predictor and validation: To validate this method, an RFC was trained using the 859 selected sites and then applied it to a separate snmC dataset comprising individual repeats and 3 unseen donors. This resulted in an accuracy of 100%.

### Example 2

### Identification of aDMRs and aDMR Interactions

Identification of aDMRs in Primary Entorhinal Cortex

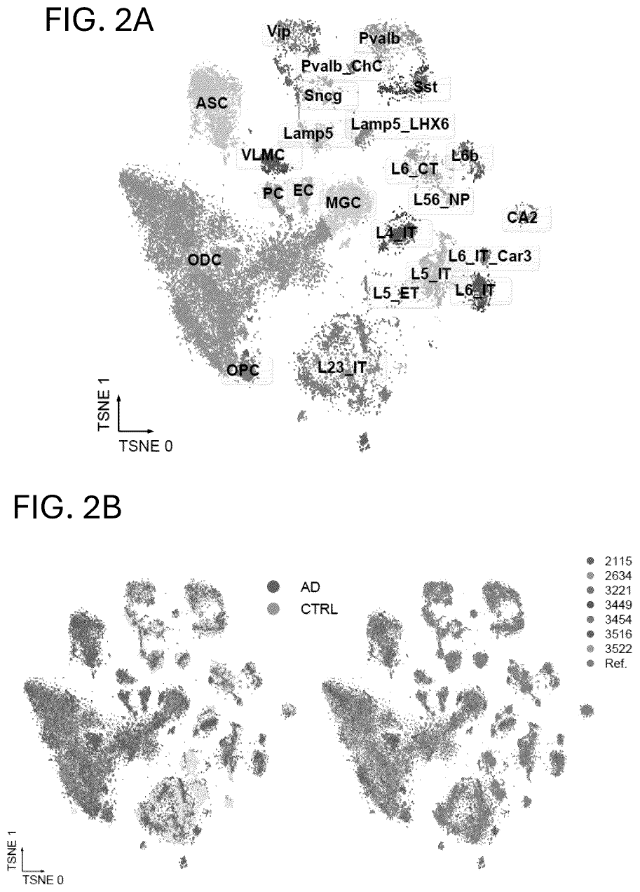

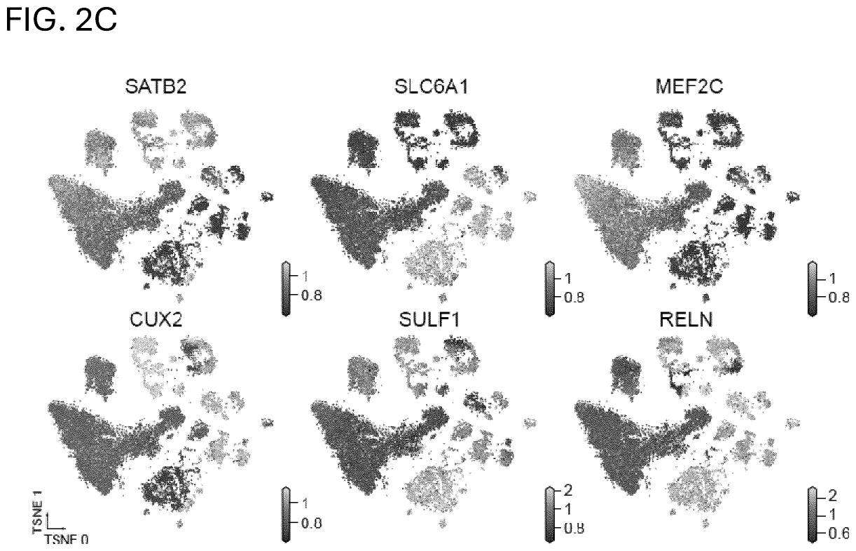

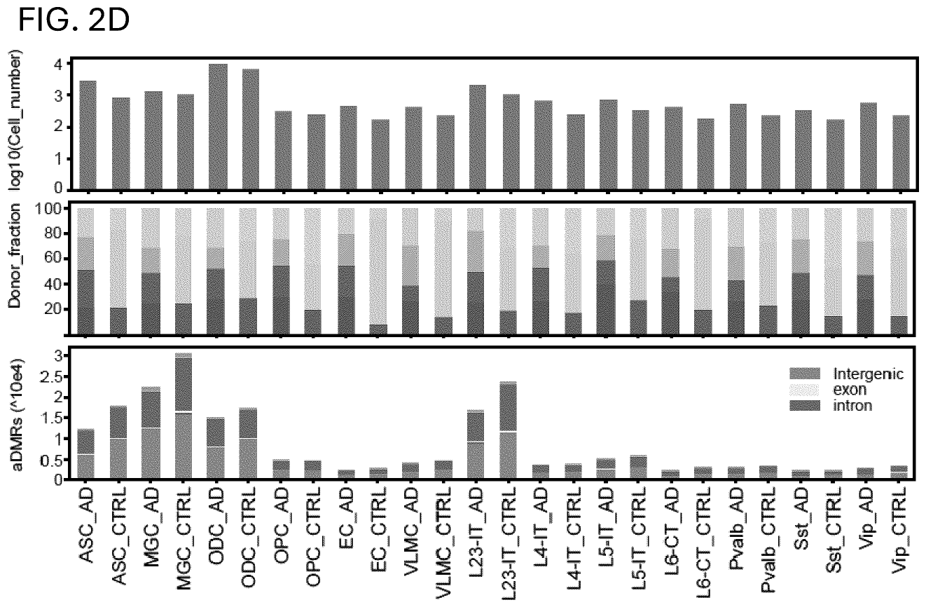

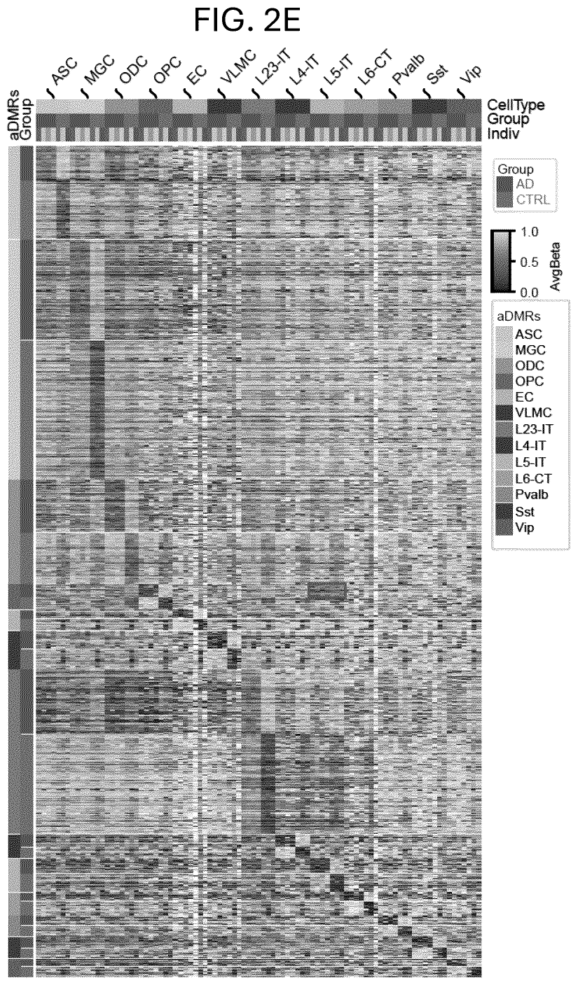

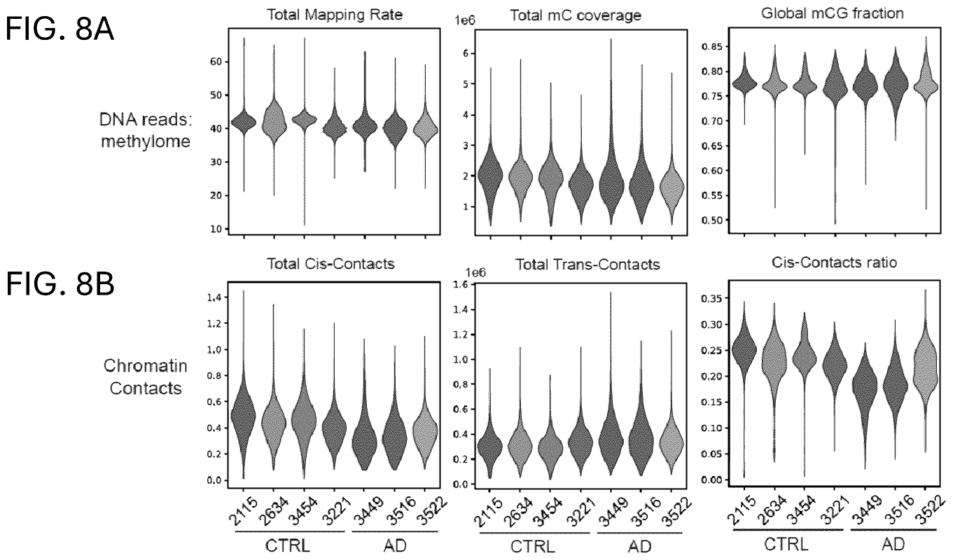

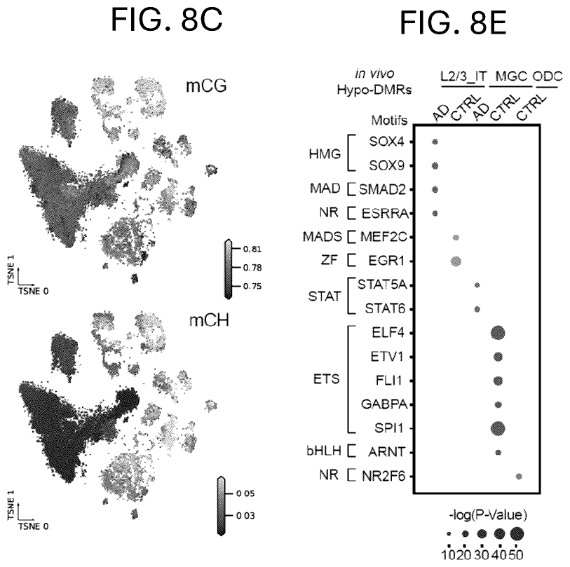

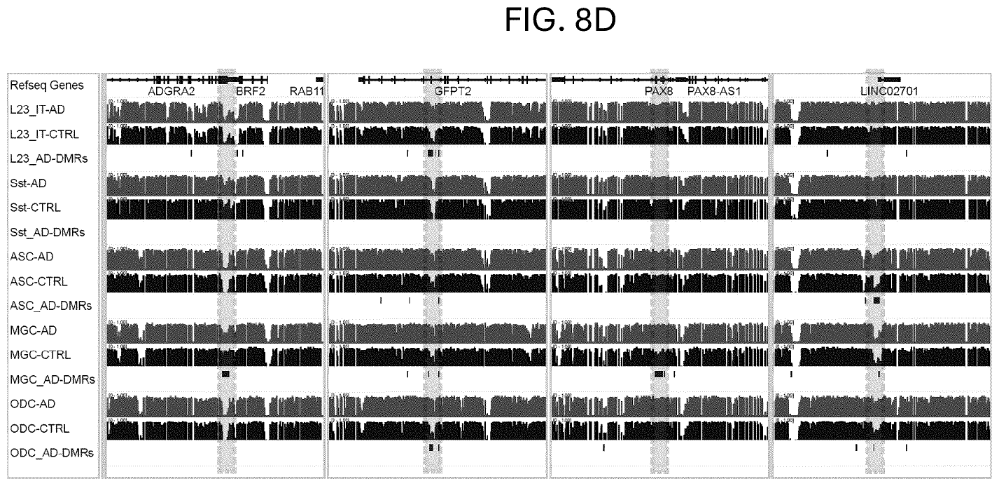

The single nucleus RNA sequencing (snRNA-seq) and ATAC sequencing (snATAC-seq) of AD brain tissues have demonstrated that AD-specific transcriptome changes strongly depend on cellular identity (Mathys et al., Nature 570, 332-337, 2019; Morabito et al., Nature Genetics 53, 1143-1155, 2021; Gabitto et al., Res Sq. 10.21203/rs.3.rs-2921860/v1, 2023; Anderson et al., Cell Genome 3, 200263, 2023). However, the alterations of DNA methylation and 3D chromatin architecture in LOAD brain cell types are still unclear. Single nucleus multi-omics technologies, snm3C-seq (Lee et al., Nat. Methods 16, 999-1006), was applied to capture the methylome and 3D chromatin conformation to 4 AD and 3 age-matched controls' (CTRL) post-mortem human entorhinal cortex, a region critical in the development of AD (Braak et al., Brain Pathol. 1, 213-216, 1991; de Calignon et al., Neuron 73, 685-697, 2012). Collectively, 34,090 nuclei passed rigorous quality control, with 2.3±0.7 million unique mapped reads and 4.3±1.4×e5 chromatin contacts detected per cell (FIGS. 8A-8B). These provided reliable quantification of methylome and detection of active or repressive chromatin compartments, topologically associating domains (TADs), and chromatin loops in distinct cell types in the entorhinal cortex. After integration with human brain methylome datasets (Tian et al., bioRxiv 2022.11.30.518285. 10.1101/2022.11.30.518285, 2022) based on mCH and mCG across 100-kb genomic bins, six cell classes were annotated, including excitatory neurons (Ex), inhibitory neurons (Inh), astrocytes (ASC), microglia (MGC), oligodendrocyte progenitor cells (OPC), and oligodendrocytes (ODC). They were further separated into 24 major cell types (FIG. 2A and Methods). Excitatory neuron clusters were separated into cortical layers (L) (L2/3, L4, L5, L5/6, and L6) and projection types (IT=intratelencephalic, CT=corticothalamic, ET=extra telencephalic, and NP=near-projecting). Inhibitory neuron types expressing different neuropeptides could be separated, including Vip, Sst, and Pvalb, as well as rarer types like Lamp5_Lhx6 and Pvalb_ChC. A small fraction of CA2 neurons might be attributable to dissection contamination of the adjacent region between the hippocampus and entorhinal cortex. No significant (p-value=0.469, t-test) cell type proportion differences were observed between 3 CTRL and 4 AD donors, with slightly increased ASC (p-value=0.021) and decreased MGC proportions (p-value=0.024) in AD individuals (FIGS. 1B and 1D). Consistent with findings in other vertebrate brain systems and young human donors (Tian et al., bioRxiv 2022.11.30.518285. 10.1101/2022.11.30.518285, 2022), 5mCs were also observed in abundance in non-CG (or CH, H=A, C, or T) contexts in aged human individuals, especially in neurons rather than glial cell types (FIG. 8C). Global methylation levels across major cell types ranged from 74.2% to 81.9% for CG-methylation and 0.8% to 8.5% for CH-methylation without obvious differences across individuals. Consistent with the previous finding, gene activation negatively correlated with gene-body mCH levels at cell-type marker genes (Tian et al., bioRxiv 2022.11.30.518285. 10.1101/2022.11.30.518285, 2022; Luo et al., Science 357, 600-604; Liu et al., Nature 598, 120-128, 2021). For example, SATB2 is a marker gene for excitatory neurons and showed reduced mCH in excitatory neuron clusters (FIG. 2C).

To identify the AD-specific putative cis-regulatory elements in a brain cell type-specific manner, individuals were grouped into AD and CTRL and paired 209,972 aDMRs in the 13 major cell types were identified. MGC, ASC, ODC, and L2/3 IT neurons had the largest numbers of aDMRs located mostly at the intergenic and intronic regions (FIG. 2D). The consistent methylation patterns of aDMRs across individuals showed robust AD/CTRL differences, most of which were cell-type specific (FIG. 2E). Meanwhile, shared aDMRs across multiple cell types were observed, like aDMRs located in gene GFPT2 introns, which were hypermethylated in AD L2/3 IT neurons and AD ODC (FIG. 8D). GFPT2 (glutamine-fructose-6-phosphate transaminase 2) is involved in the glutamate metabolism pathway and controls the flux of glucose, which is becoming increasingly recognized as a hypometabolism phenotype in cancer and AD brain (Kim et al., Nat Metab 2, 1401-1412, 2020; Kyrtata et al., Neurosci. 15, 626636, 2021). In addition, the TF motif enrichments (−log(p-value) >10) in aDMRs across cell types was analyzed (FIG. 8E). For example, the TF SPIl enriched in aDMRs hypomethylated in CTRL microglia was identified. The hypermethylated state of these aDMRs in AD microglia reflects the transcriptional repression in SPIl target genes and is consistent with AD snATAC-seq analysis (Morabito et al., Nature 53, 1143-1155, 2021). Furthermore, the aDMRs distribution was quantified across the genome and 1,795 hotspots enriched for aDMRs across the autosomes were identified with 26% of these hotspots shared between at least 2 major cell types (FIG. 2A). The genome feature annotations of these hotspots showed a significant enrichment of CpG island and SINE-VNTR-Alu (SVA) class of retrotransposons (FIG. 2B). Intriguingly, SVA as the evolutionarily young and hominid-specific retrotransposons and LINE1 are mobilized active in the human genome and are involved in human neurodegenerative diseases (Ostertag et al., Am. J. Hum. Genet. 73, 1444-1451, 2003; Ravel-Godreuil et al., FEBS Lett. 595, 2733-2755).

aDMRs Interact with Bivalent Promoters of AD Differential Expression Genes

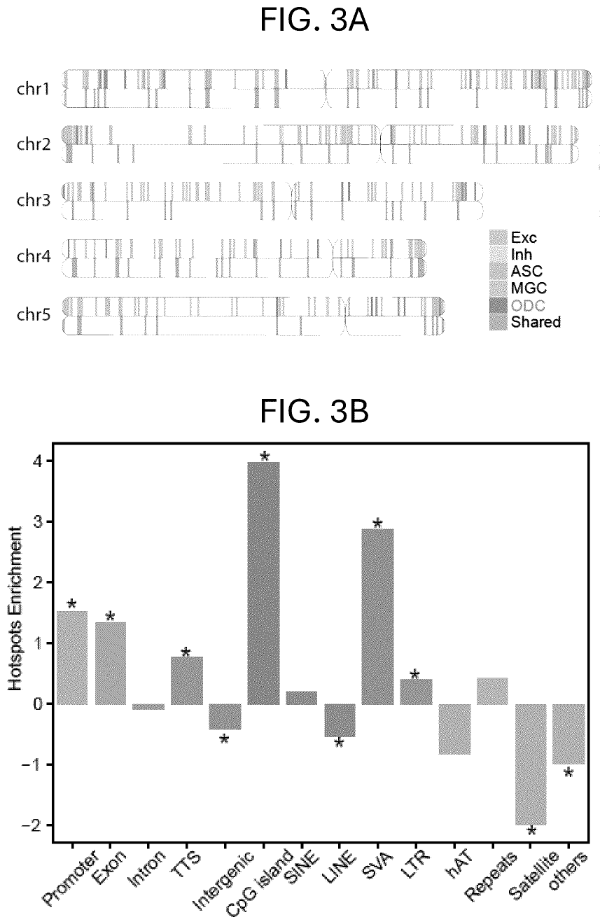

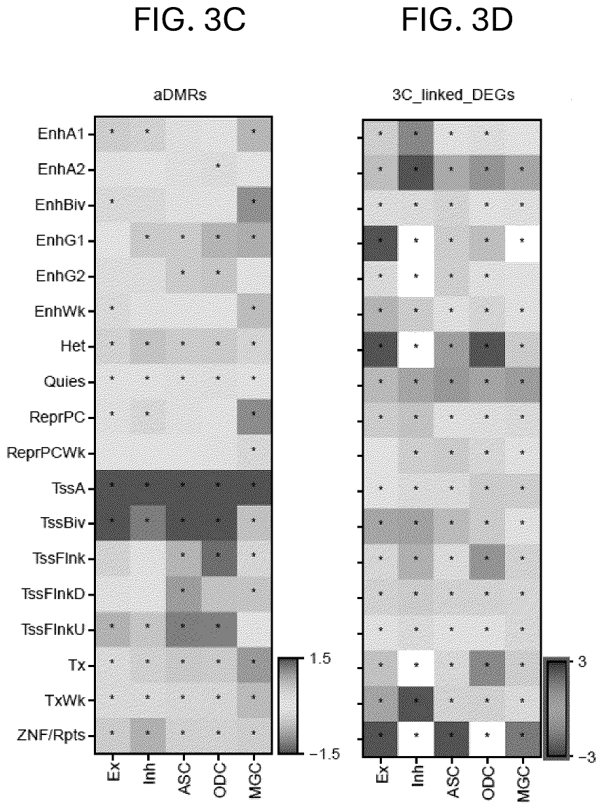

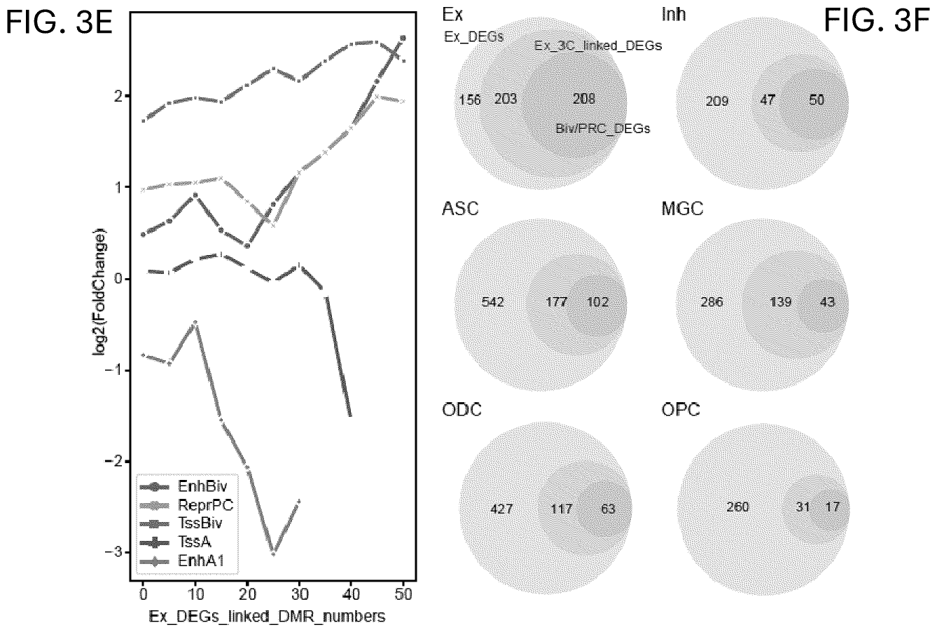

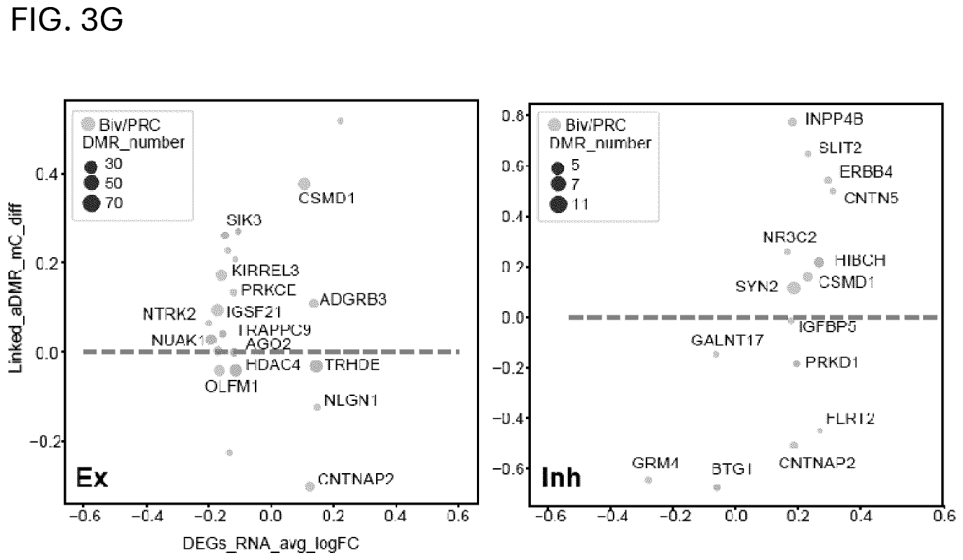

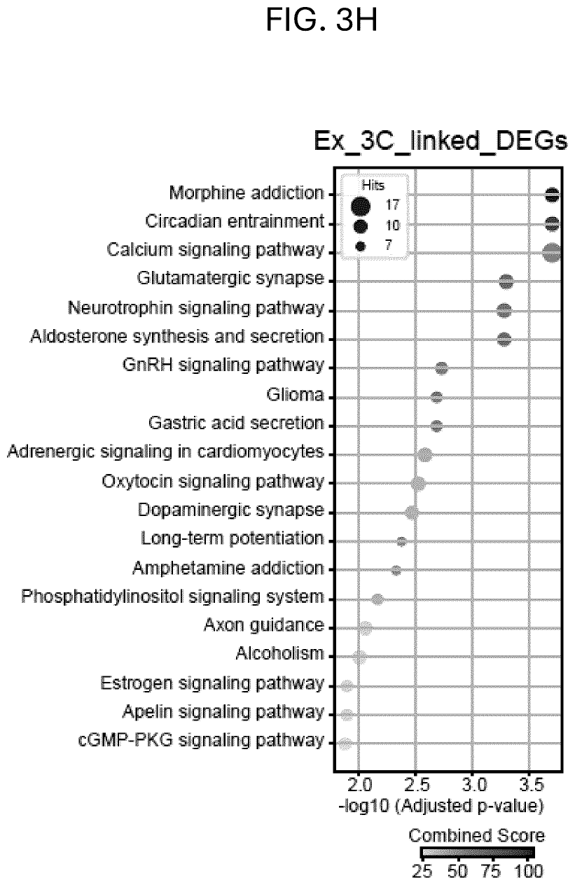

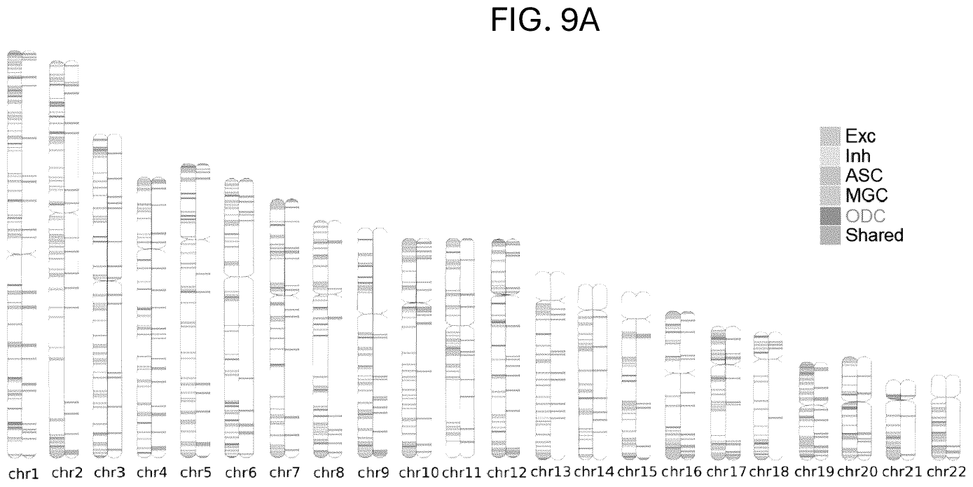

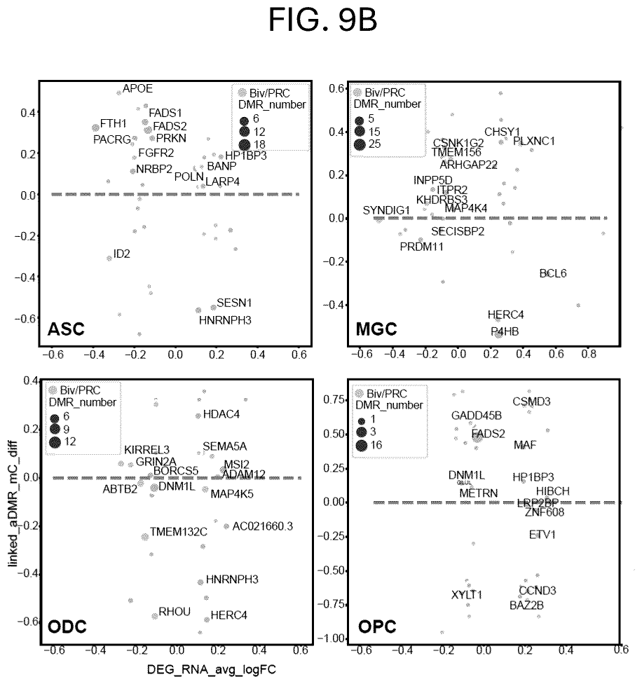

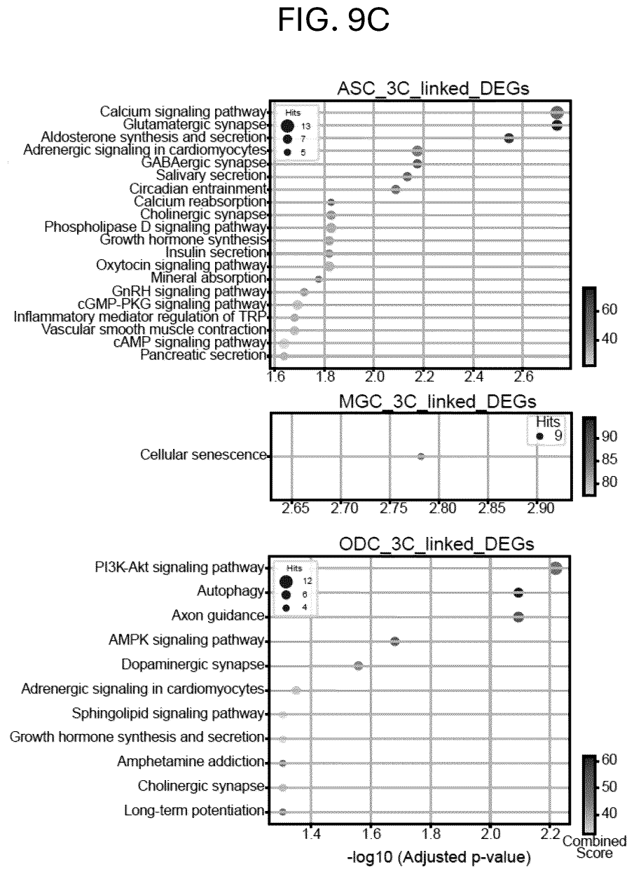

To interrogate the multivalent interactions regulated by DNA methylation and 3D genome structures on transcriptional activity, putative cis-regulatory elements (CREs) of differential expression genes (DEGs) identified in distinct cell types of snRNA-seq dataset (Morabito et al., Nature 53, 1143-1155, 2021) by assigning the aDMRs to genes based on the loop interaction were identified. In total, 6,214 aDMRs/DEGs pairs, between 1,197 DEGs (across six major cell types) were assigned with 5,345 aDMRs in corresponding cell types. A significant enrichment of aDMRs was found at heterochromatin (Het) and zinc finger protein genes associated with chromatin states (Znf/Rpts) (Roadmap Epigenomics Consortium et al., Nature 518, 317-330, 2015; Ernst et al., Nat. Methods 9, 215-216, 2012) (FIG. 3C). These repressive states were usually marked by H3K9me3, associated with lamin-associated domains (LADs) and B compartments (Pickersgill et al., Nat. Genet. 38, 1005-1014, 2006; Leibermann-Aiden et al., Science 326, 289-293). However, the chromatin states on the promoters of DEGs linked with aDMRs across cell types show significant enrichment of repressed Polycomb states (ReprPC) and bivalent regulatory states (TssBiv and EnhBiv) (FIG. 3D). The increased number of aDMRs linked to DEGs in excitatory neurons amplified the enrichment of these repressive states, like TssBiv, with depletion of active promoters (TssA1 and EnhAl) (FIG. 3E). Bivalent promoters are usually marked both with active (H3K4me3) and repressive (H3K27me3) histone modifications (Bernstein et al., Cell 125, 315-326, 2006). Approximately 15% of the promoters of total coding genes exhibit repressive bivalent states or are bound by the PRC complex in the human brain. This proportion surged to 50% for the promoters of DEGs linked with aDMRs in both excitatory and inhibitory neurons, as well as modest increments in glial cells (36% in ASC, 35% in ODC, and 24% in MGC) (FIGS. 3F-3G). Examination of the combinatorial interactions between RNA expression of DEGs and average methylation alterations of linked aDMRs showed no clear correlation, suggesting a more complex relationship between epigenetic regulation and gene expression (FIG. 3G). aDMRs linked DEGs in excitatory neurons are enriched in KEGG pathways related to glutamatergic synapse and axon guidance processes (FIG. 3H). For example, one of the top genes linked with aDMRs in neurons, CSMD1 (CUB and Sushi Multiple Domains 1) was associated with 68 aDMRs in excitatory neurons and 10 aDMRs in inhibitory neurons and had bivalent promoter and upregulated RNA expression in AD samples (FIG. 3G). A genetic variant of CSMD1 was previously found to be associated with schizophrenia and may be involved in the complement cascade system, including synaptic pruning and neuroinflammation (Lam et al., Nat. Genet. 51, 1970-1678, 2019; Baum et al., BIoRxiv, 2020.09.11.291427. 10.1101/2020.09.11.291427, 2020). For certain DEGs in other cell types, like APOE in ASC, eight aDMRs associated with the gene were identified, and seven of them were hypermethylated in AD samples, consistent with the downregulation of APOE RNA expression. An upregulated AD DEGs example in microglia and astrocytes, BCL6, had 12 aDMRs (MGC) and 5 aDMRs (ASC) linked via loop interactions. Eight of the twelve aDMRs interacting with the BCL6 gene in microglia were located in gene TPRG1 (Tumor Protein P63 Regulated 1), which is known to contain differentially methylated loci in epigenome-wide association studies (EWAS) of AD (Li et al., Clin. Epigenetics 12, 149, 2020; Smith et al., Nat. Commun. 12, 3517, 2021). One upregulated AD DEGs found in neurodegenerative disease and AD mouse models (Wu et al., Front. Mol. Neuroscie. 9, 114, 2016), HDAC4 (Histone Deacetylases 4) was upregulated specifically in ODC and interacted with 6 aDMRs spanning from 354 to 826 kbps downstream of the transcription start site (TSS), with three of them being hypomethylated in AD and three hypermethylated (FIGS. 9A-9C). In summary, this comprehensive datasets provide valuable resources to analyze the interplay between cis-regulatory elements and genes involved in AD pathogenesis, which enables the characterization of the associations of aDMRs with bivalent promoters and PRC repressive elements within DEGs.

### Example 3

### Chromosomal Epigenome Erosion in AD Brain Cell Types

Chromatin is organized into structures at different scales. The subchromosomal-level compartment brings together regions that are tens to hundreds of megabases (Mb) away, whereas TADs and chromatin loops are driven by interactions within several Mb. Chromatin organization and related dysfunctional nuclear lamina (LMNA) in Hutchinson-Gliford Progeria have demonstrated the critical role of chromatin architecture in senescent cells, normal aging, and age-dependent disorders (Yang et al., Cell 186, 305-326, 2023; Liu et al., Nature 472, 221-225, 2011; Chandra et al., Cell Rep. 10, 471-483, 2015; López-Otin et al., Cell 186, 243-278, 2023). The initial studies in neurodegeneration mouse models (Parkinson's disease and AD) suggested abnormal dysfunctional histone modifiers such as SIRT and HDAC family (Bhatt et al., Int. J. Neurosci., 1-26, 2022; Graff et al., Nature 483, 222-226, 2012). Further studies on heterochromatin protein 1α (HP1α), Polycomb group proteins, and ATP-dependent chromatin remodeler-like CHD5 indicated that the disrupted chromatin structures and organization contributed to aging and age-related neurodegenerative disorders (Larson et al., PLoS Genet. 8, e1002473, 2012; El Hajjar et al., Sci. Rep. 9, 594, 2019; Esposito et al., Front. Neurosci. 13, 476, 2019). Chromatin accessibility assays in bulk (ATAC-seq) showed that the AD-associated cis-regulatory domains were enriched in A compartments (Bendl et al., Nat. Neurosci. 25, 1366-1378, 2022). However, the 3D genome architecture and DNA loop contact maps in the LOAD brain, especially in distinct cell types, are still unknown.

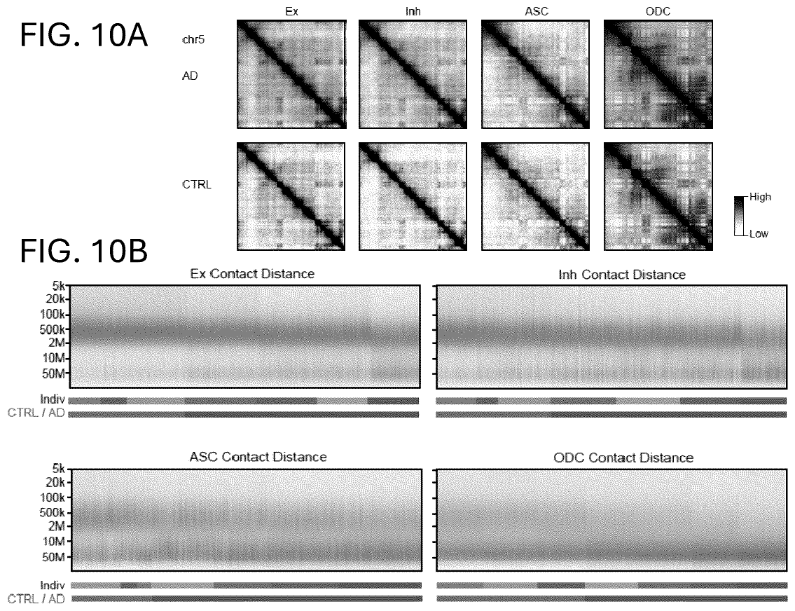

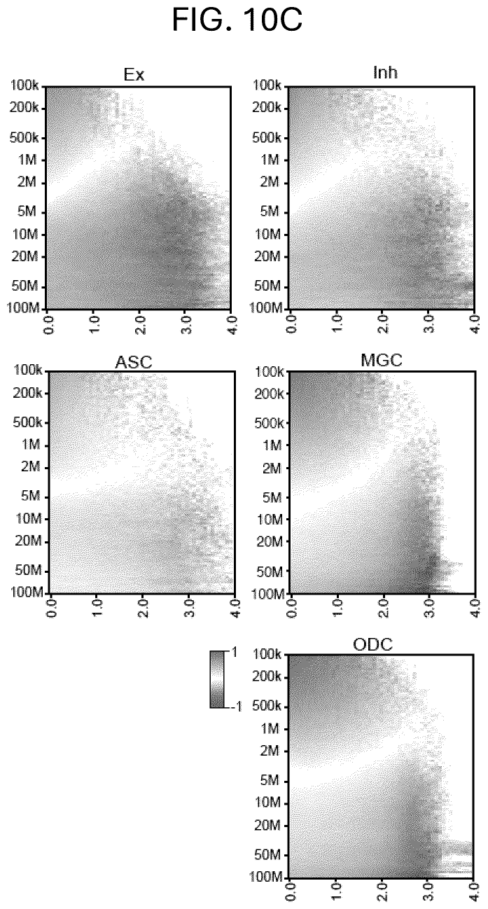

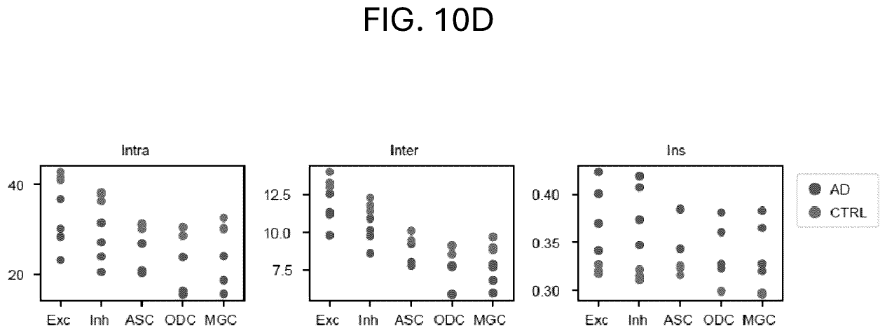

The proportion of contacts detected at different genome distances within each single cell was first determined to examine the cell-type specificity of genome folding at different length scales. Within the same cell type, AD samples have significantly more longer-range interactions (20-50 Mb) and fewer shorter-range interactions (200 kb-2 Mb) compared to CTRL samples (FIGS. 4A-4C, 10A-10B). Given that megabase-level interactions are usually associated with compartment organization, the relationship between enriched, longer-range chromatin contacts and chromatin compartments was investigated. The chromosome compartments in each cell type at 100 kb resolution was identified and it was observed that enriched longer-range interactions in AD were predominantly inter-compartmental (FIG. 10C). This finding differed from the enrichment of longer-range contacts seen in non-neural cells compared to neurons, where intra-compartment interactions dominated (Tian et al., bioRxiv 2022.11.30.518285. 10.1101/2022.11.30.518285, 2022). Consistently, the compartment strength was weakened in AD, as quantified by the decrease in contact correlation between the intracompartment regions and the ratio between intracompartment and intercompartment interactions (FIGS. 4D-4F, 10D). The domains at 25 kb resolution and loops at 10 kb resolution were also identified. The number of identified domains decreased in AD cells of all cell types (FIG. 4G), whereas the number of loops significantly was reduced in AD samples only in ODC and MGC (FIG. 4H). Generally, the insulation score at domain boundaries and the interaction strength at chromatin loops were weaker in AD (FIGS. 10D-10E), consistent with the decreased shorter-range interactions. To associate the chromatin structure with gene expression, 43,620 differential loops (DL) between AD and CTRLs were identified, with nearly all DL being lost in AD (99.8% of total DL). For example, TMEM59 is a transmembrane protein inhibiting APP transportation to the cell surface and downregulated in AD inhibitory neurons. This data suggested that the chromatin loops surrounding the genes were also impaired during AD, and the DMRs associated with TMEM59 by chromatin loops also showed increased mCG (FIG. 4G), suggesting the potential epigenetic dysfunction that led to the misregulation of its expression. Together, this data indicated that an erosion process of chromatin occurs in AD, which leads to the erroneous expression of bivalent repressive and PRC binding genes (FIGS. 4I-4J).

### Example 4

### Epigenetic Signatures can be Detected in Cellular Models

snmCT-Seq Characterization of Distinct Cell States in Human Neurons Directly Converted from AD Fibroblasts

Modeling age-dependent neurodegenerative diseases is by far one of the biggest challenges for researchers seeking to find cellular model systems or animals that can recapitulate temporal dynamics of up to years in duration. Reprogramming patient tissues to induced pluripotent stem cells (iPSCs) is a powerful approach for genetic-based disease modeling; iNs can be generated through differentiation from reprogrammed iPSCs or by direct conversion from patient somatic fibroblasts (Zhang et al., Nat. Biotechnol. 19, 1129-1133, 2001; Vierbuchen et al., Nature 463, 1035-1041, 2010). However, the transition through the stem cell intermediate phase leads to a youthful rejuvenation of the epigenome (FIGS. 11A-11J), gene expression, long-lived proteins, mitochondria function, and telomere length in the resulting neuron (Maherali et al., Cell Stem Cell 1, 55-70, 2007; Miessner et al., Nature 454, 766-770, 2008; Huh et al., Elife 5. 10.7554, 2016; Mertens et al., Annu. Rev. Genet. 52, 271-293, 2018; Scafer et al., Nat. Neurosci. 22, 243-255, 2019). The directly reprogrammed neurons from fibroblasts retain biological aspects of age and disease (Mertens et al., Annu. Rev. Genet. 52, 271-293, 2018; Mertens et al., Cell Stem Cell 17, 705-718, 2015; Mertens et al., Cell Stem Cell. 10.1016/j.stem.2021.04.004, 2021; Herdy et al., Cell Stem Cell 29, 1637-1652, 2022; Traxler et al., Nat. Rev. Neruol. 10.1038/s41582-023-00815-0, 2023). Single-cell RNA-seq studies in mouse iNs directly converted from fibroblasts have suggested there is cell state diversity during transdifferentiation (Treutlein et al., Nature 534, 391-395, 2016). Genome-wide methylation and open chromatin dynamics revealed epigenome and chromatin reconfiguration during mouse direct reprogramming (Wapinski in et al., Cell Rep. 20, 3236-3247, 2017; Luo et al., Elife 8. 10.7554/eLife.40197, 2019). However, the heterogeneity and epigenome dynamics of iN conversion of human fibroblasts, especially from aged patients and AD donors, have yet to be investigated.

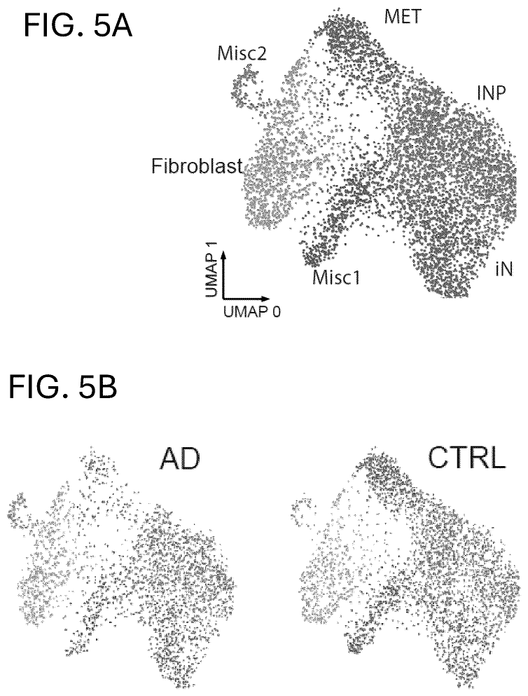

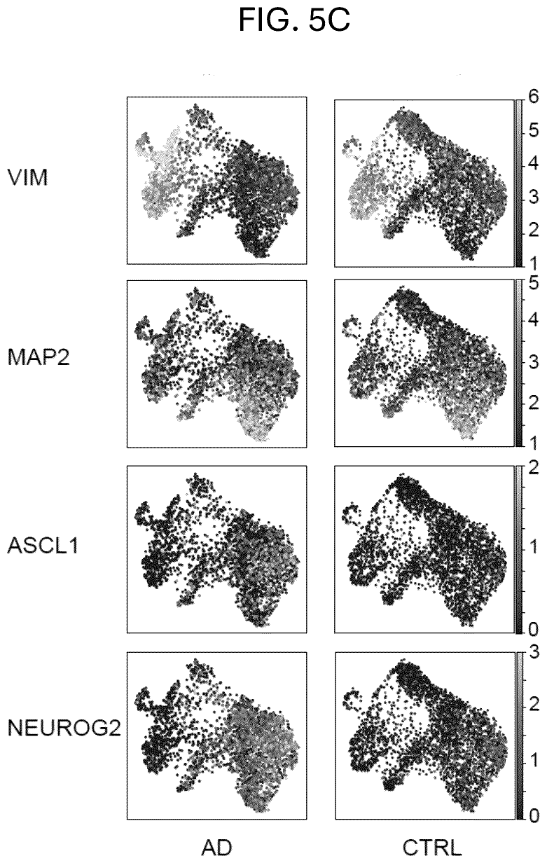

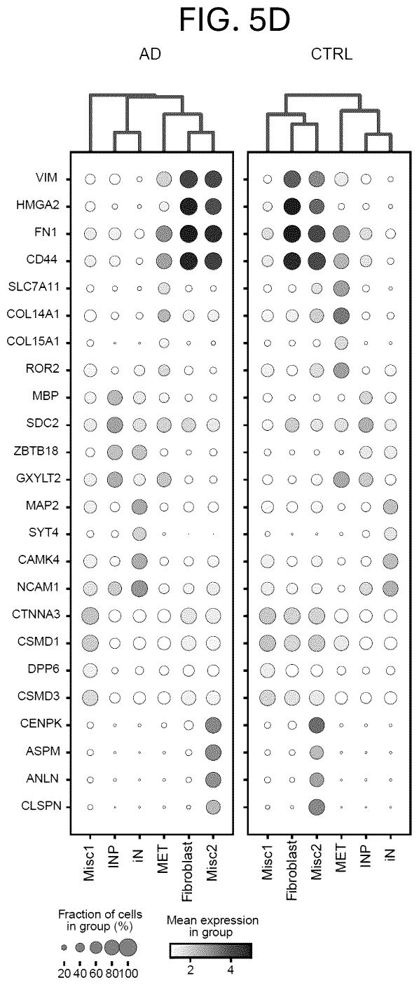

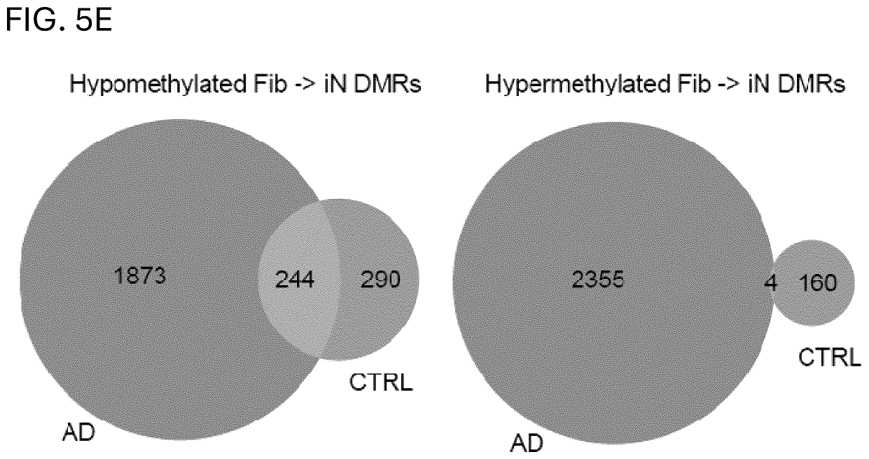

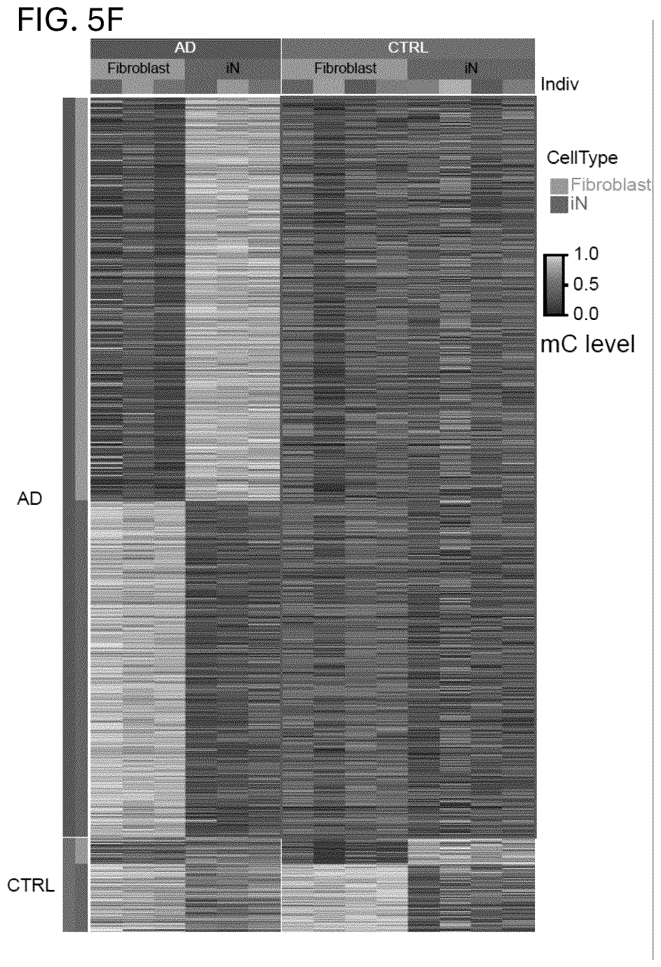

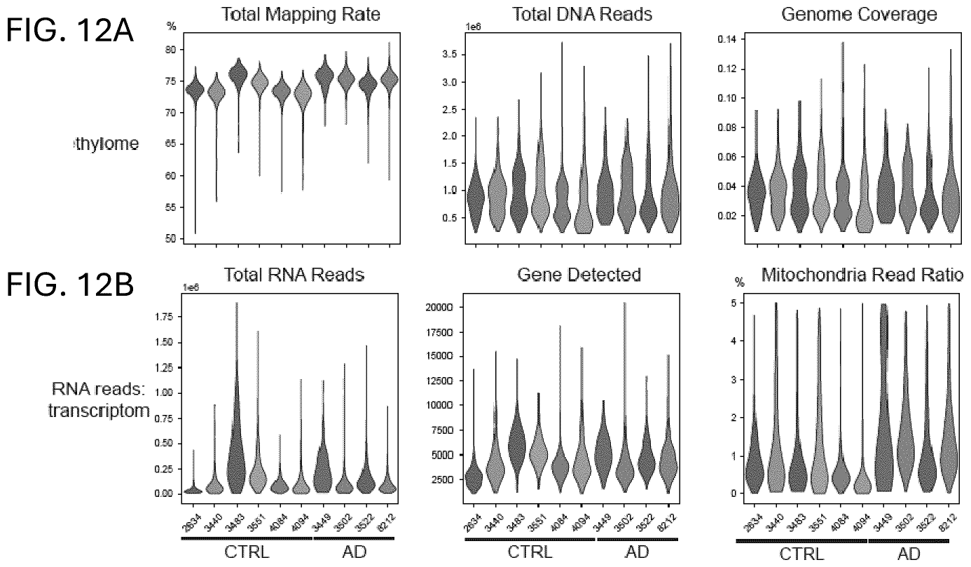

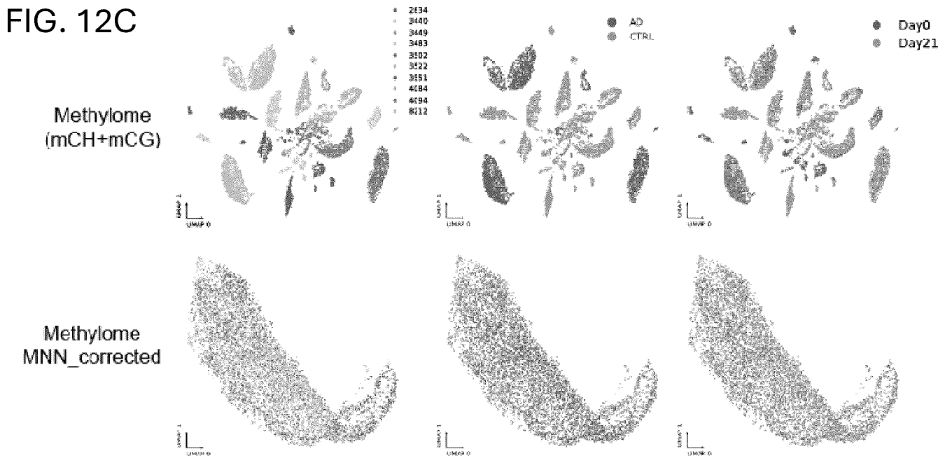

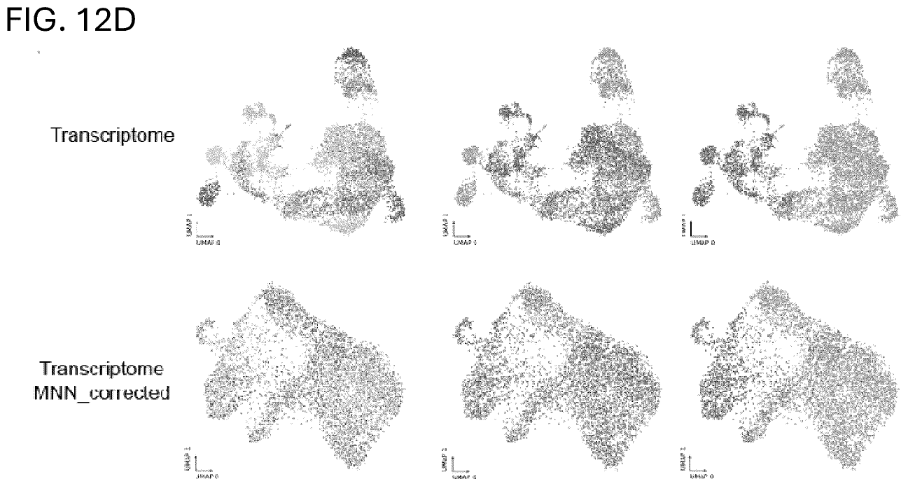

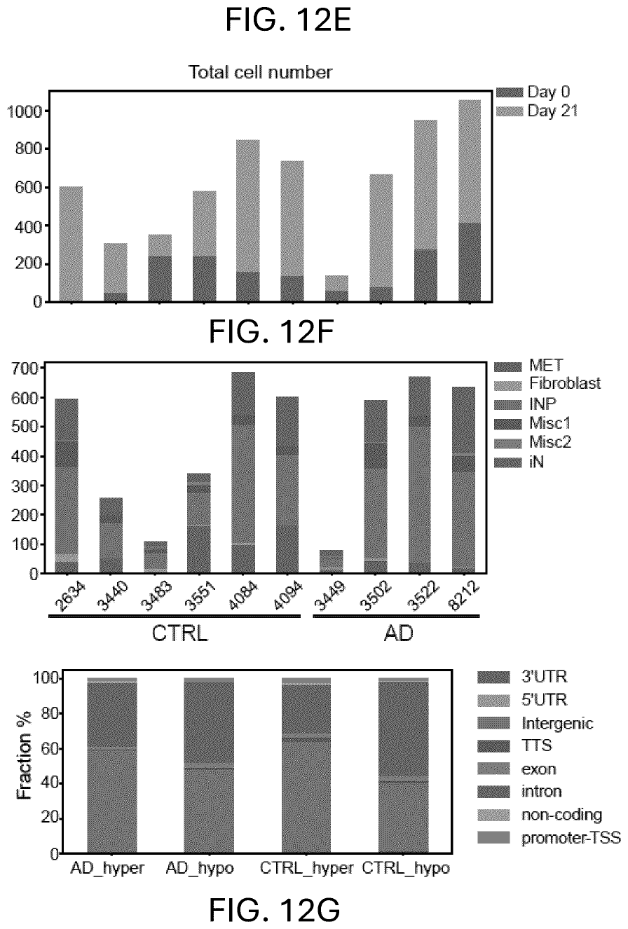

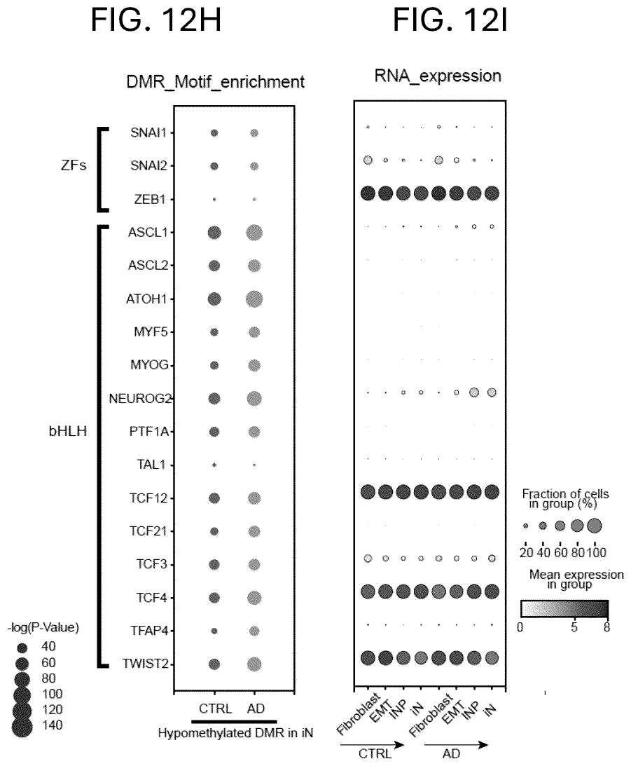

To characterize the cell state transitions along the human fibroblast-to-iN conversion process and evaluate whether direct trans-differentiation iN models can mimic aging and AD signatures in primary brain tissues, iNs from 6 LOAD and 4 age-matched were profiled, cognitively normal control individuals, generating a snmCT-seq dataset of 6,242 cells as well as a snmC-seq dataset of 11,402 cells. The cells were not sorted with PSA-NCAM because snmCT-seq was used and can identify cells during analysis. The data quality is comparable to previous work (Luo et al., Cell Genom 2.10.1016/j.xgen.2022.100107, 2022) (FIGS. 12A-12B). The methylome modality in the snmCT-seq dataset covered 3.47%±1.7% (mean±s.d.) of the genome, whereas the transcriptome detected 4,273±1726 genes from each single nucleus. Based on transcriptome clustering, 6 distinct cell states were identified during the iN-induction process: fibroblast, mesenchymal to epithelial transition (MET), intermediate neuronal progenitor (INP), iN, Misc1 and Misc2 (FIGS. 5A-5B). The downregulated expression of fibroblast marker genes (VIM, FN1, and CD44) and upregulated expression of neuron marker genes (MAP2, NCAM1, and CAMK4) from fibroblast to iN state confirmed the successful iN conversion in both AD and CTRL individuals (FIGS. 5C-5D). The diversity of conversion efficiency across individuals can be quantified by the proportion of iN states after 3 weeks of the iN-induction process, varying from 22%±3% in CTRL and 23%±11% in AD (FIGS. 12A-12F). Misc1 and Misc2 clusters have mixed marker genes and are labeled as uncharacterized cell states. Misc1 may be due to transition failure during the transdifferentiation since several genes within heterochromatin are active, like CSMD gene families. In contrast, cell cycle-related genes like CENP family genes and DNA replication genes are highly expressed in the Misc2 cluster, suggesting it may represent cells in oncogenesis or senescence.

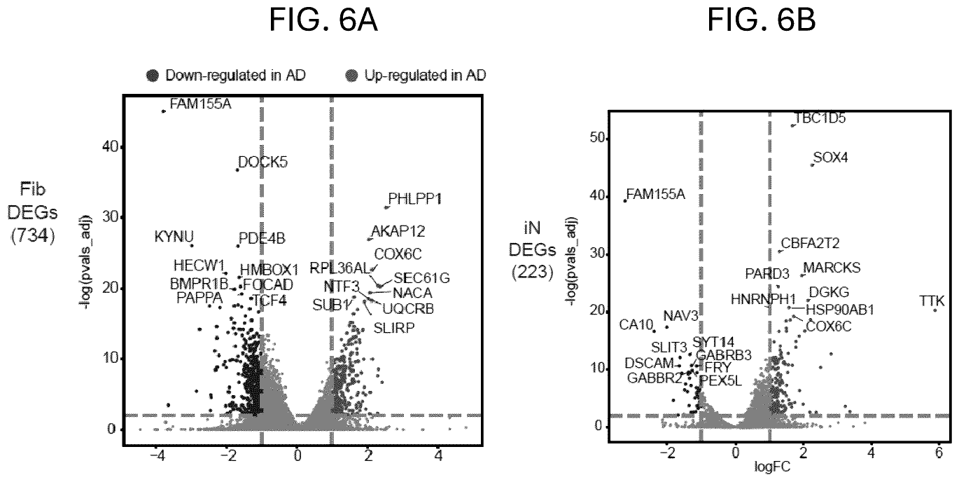

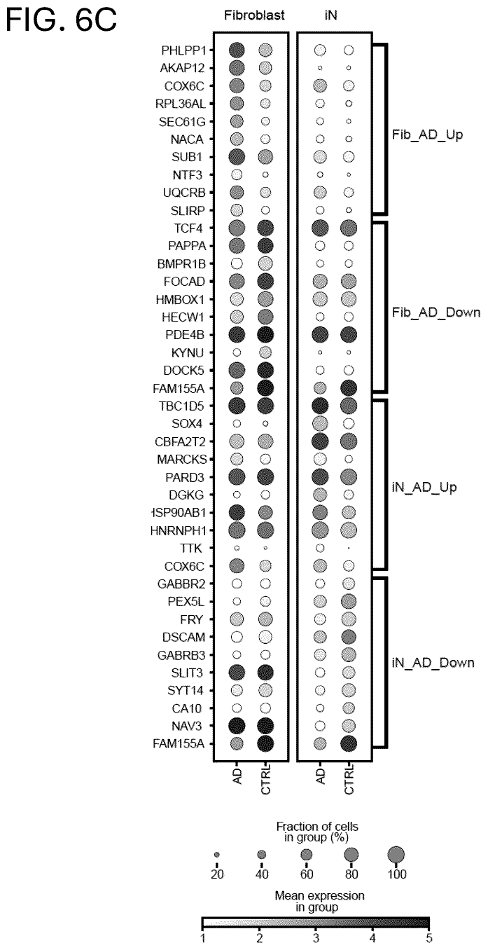

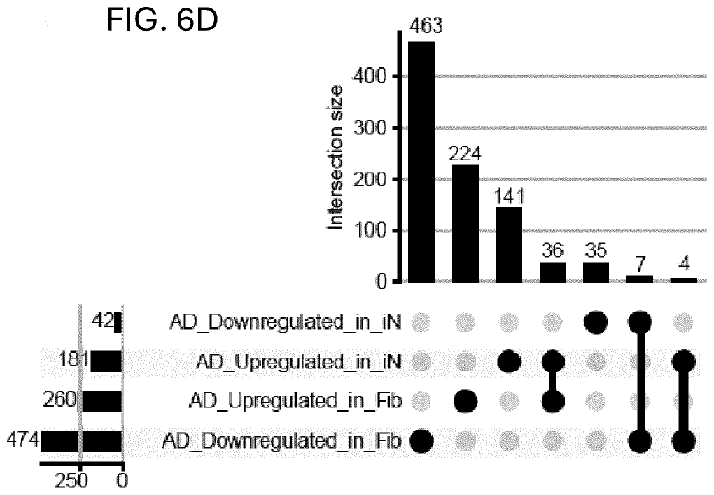

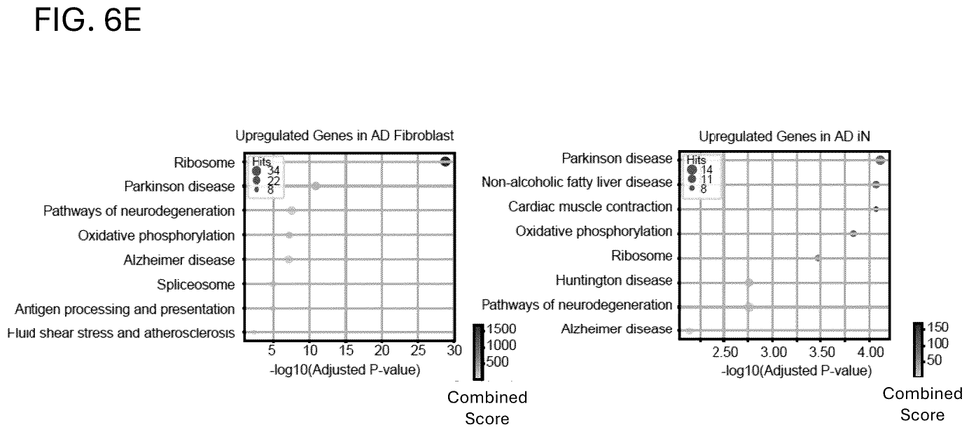

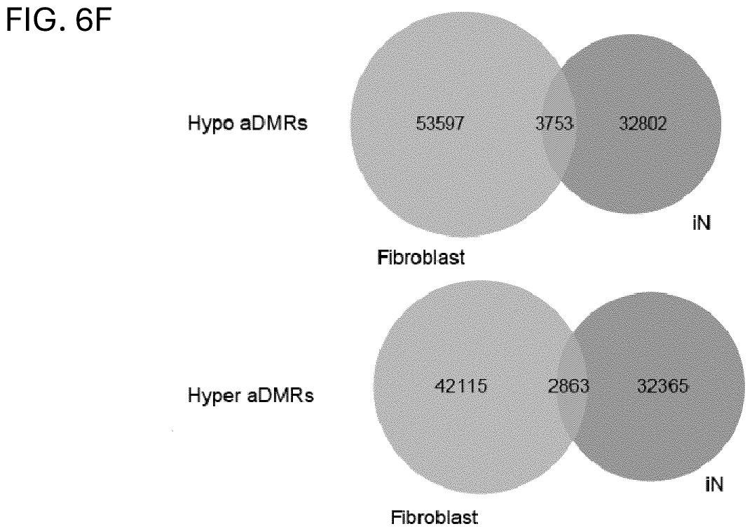

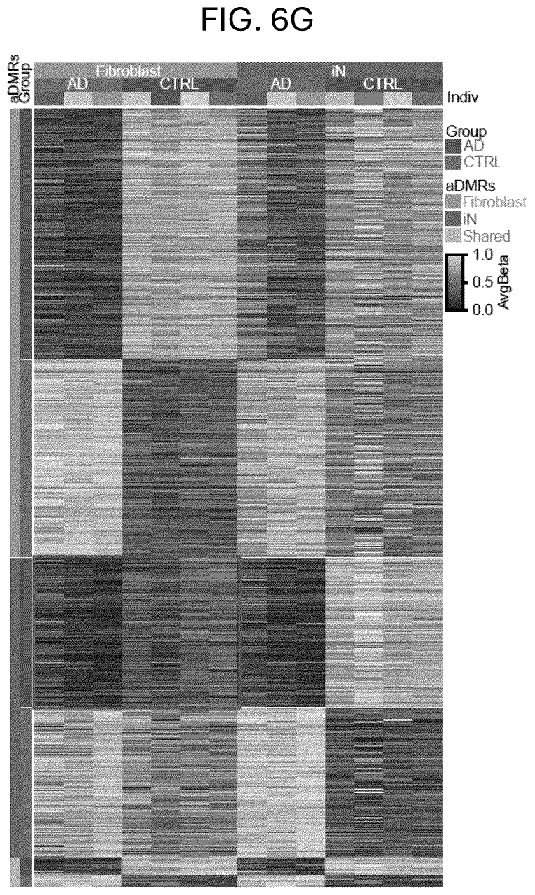

To examine methylome reconfiguration during iN induction, fibroblasts were compared within populations from the same individuals, i.e., CTRL and AD cells were compared separately. In total, 4,476 (AD) and 698 (CTRL) fibroblast ->iN conversion-related DMRs (cDMRs) were identified and were consistent across individuals within the AD/CTRL groups (FIG. 6E). 244 cDMRs were observed that were shared between AD and CTRL groups, and almost all of them had methylation levels decreased from fibroblast to iN, suggesting that the overexpression of ASCL1/NGN2 neuronal factors initiated a shared demethylation program at downstream targets regardless of disease status. Conversely, only 4 cDMRs that gained methylation during iN induction were shared between AD and CTRL. Overall, cDMRs were in intergenic (51.67%±11.08%) and intronic (41.19%±11.38%) genome features (FIG. 12G). The motif enrichment analysis of cDMRs by HOMER (Heinz et al., Mol. Cell 38, 576-589, 2010) revealed a shared demethylation pattern surrounding the binding sites of Zinc fingers (ZFs) and basic Helix-Loop-Helix (bHLH) TFs; there was no significant (p-value <0.01) motif enrichment at hypermethylated regions along the iN conversion trajectory (FIG. 12H). Intriguingly, neuronal differentiation genes like TCF4 coupling with induction factors ASCL1 and NGN2 showed increased RNA expression and their binding motifs are enriched at the cDMRs demethylated during the iN conversion process (FIG. 12I). Additionally, a higher expression level of ASCL1 and NGN2 and more cDMRs found during AD iN induction was observed, suggesting that the epigenetic landscape of AD fibroblasts is more permissible for iN transdifferentiation. In summary, both AD and CTRL fibroblast-derived iNs can be generated successfully with a shared ASCL1/NGN2-initiated demethylation process, and 6 cell states can be observed both in AD and CTRL during transdifferentiation.

### AD-Specific Methylation and Transcriptome Signatures in Fibroblast-Induced Neurons

Next, AD versus CTRL groups were compared within fibroblast and iN states to identify AD-specific transcriptomic and methylation signatures. To this end, 734 DEGs (p-value <0.01, log 2 foldchange >1) were identified between AD and CTRL in fibroblasts and 223 in iNs. Thirty-six DEGs were upregulated in AD in both fibroblast and iN states (shared DEGs; FIGS. 6A-6D). It was observed that 9 of the 36 shared DEGs upregulated in AD belonged to the ATP synthesis pathway in mitochondria, for example, UQCRB and COX6C. One of 7 AD-downregulated shared DEGs, FAM155A, was also downregulated in AD in endothelial cells (Sun et al., bioRxiv, 2022.02.09.479797. 10.1101/2022.02.09.479797, 2022). Strikingly, GABA receptors (e.g., GABBR2 and GABRB3) together with synaptic transmission-related genes, KCND2 and SYT14, were only observed to be downregulated in AD in iNs rather than fibroblast (FIG. 6C). KEGG pathway enrichment analysis revealed that the pathways related to neurodegeneration were enriched in AD-upregulated genes both in fibroblasts and iNs. In contrast, genes involved in synaptic transmission and neuron development were significantly downregulated only in AD iNs (FIG. 6E).

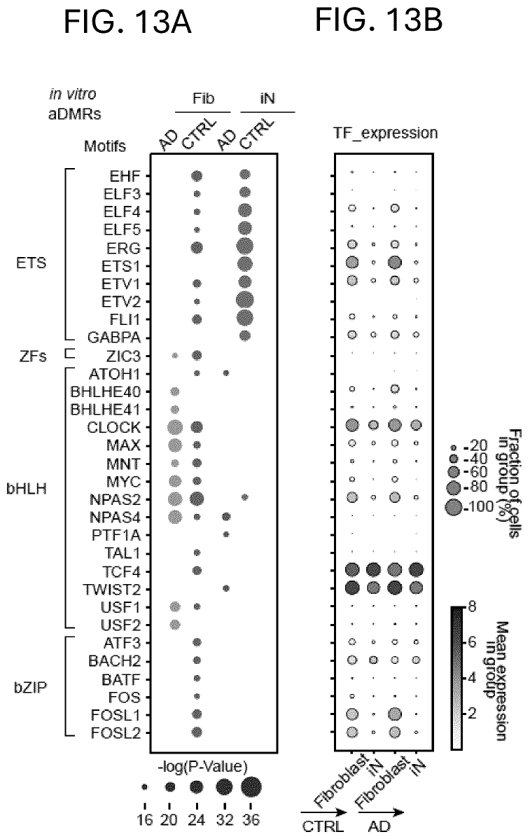

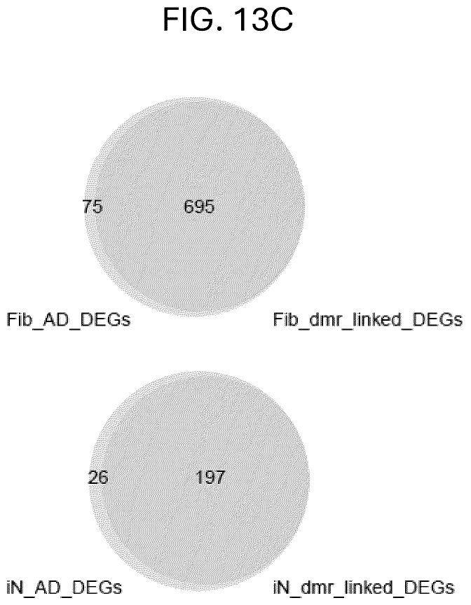

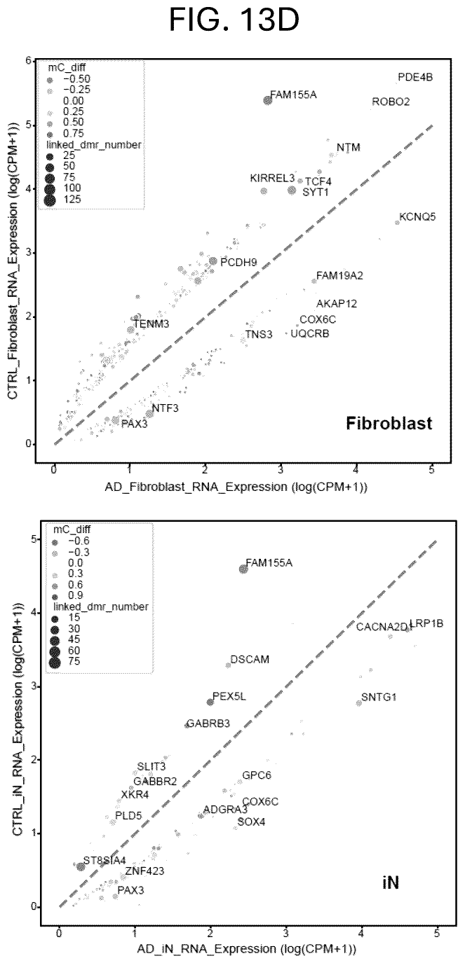

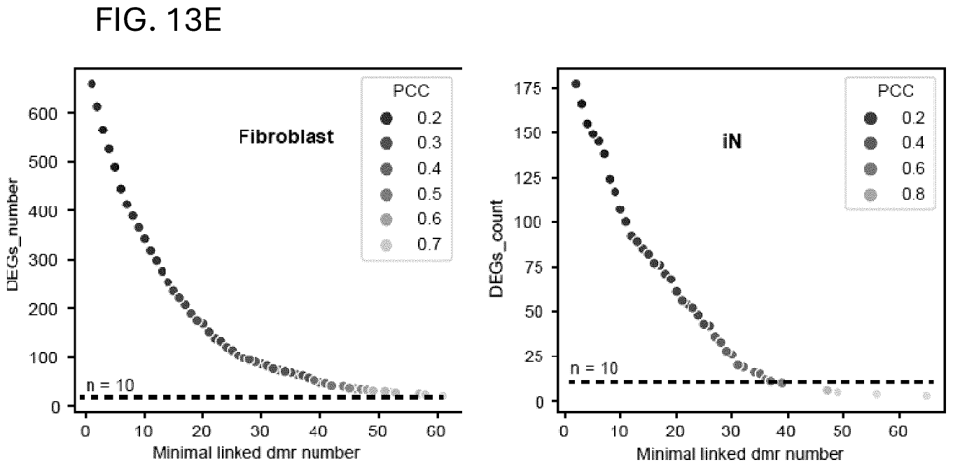

Regarding the methylome, 160,879 aDMRs were identified, most distinct in either fibroblast or iN. Overall, 3,753 aDMRs hypomethylated (2,863 hypermethylated) were identified in AD shared between fibroblast and iN states (FIG. 6F). These aDMR patterns depicted distinct DNA methylation signatures between AD and CTRL for fibroblast and iN identities (FIG. 6G). To gain insights into the potential impact of DNA methylation on the binding of TFs, a motif analysis on these aDMRs was conducted, revealing 4 top TF families (ETS, ZFs, bHLH, and bZIP) with a significant score (−log(p-value) >15). Most ETS TFs were assigned to hypomethylated DMRs in CTRL fibroblasts and iNs. In contrast, bHLH TF enrichments were specific to fibroblast cell states, and bZIP TF enrichments were specific to aDMRs hypomethylated in CTRL fibroblasts (FIG. 13A). By examining the RNA expression of these TFs from the same cells, the putative TF candidates were narrowed within each TF family and dissected the interactions between expressing TF and methylation states of their binding motifs (FIG. 13B). For instance, the AD upregulated TFs, BHLHE40 and its close homolog BHLHE41, have specific enrichments at hypomethylated aDMRs in AD fibroblast, consistent with their binding preference of unmethylated CpG sequences (Yin et al., Science 356, 10.1126/science.aaj2239, 2017). Both TFs are crucial in the regulation of cholesterol clearance and lysosomal processing in microglia and may be associated with AD pathogenesis (Hou et al., Mol Neurodegener. 17, 84, 2022; Podlesny-Drabiniok et al., bioRxiv, 2023.02.13.528372. 10.1101/2023.02.13.528372, 2023)

Furthermore, information on DEGs and DMRs was integrated to identify putative CREs in fibroblasts and iNs. DMRs were associated with genes by GREAT algorithms (McLean et al., Nat. Biotechnol. 28, 495-501, 2010). In total, 10,070 aDMRs were paired with 659 DEGs in fibroblasts, and 2,963 aDMRs were identified associated with 197 DEGs in iNs. The RNA expression changes of these DEGs and methylation alteration of the associated aDMRs revealed an orchestrated gene regulation program between AD and CTRL in vitro cellular models (FIGS. 13C-13D). For example, the downregulated shared DEG, FAM155A, was associated with 130 aDMRs in fibroblast and 83 aDMRs in iN that were hypomethylated in AD on average. In contrast, 63(35) aDMRs in fibroblast (iN) assigned to the up-regulated PAX3 gene were hypermethylated in AD samples. Concordantly, EWAS of AD has reported PAX3 to be a hotspot, showing hypermethylation in AD hippocampus (Altuna et al., Clin. Epigenetics 11, 91, 2019). Systematic examination of RNA fold changes of DEGs and methylation difference of associated aDMRs revealed a positive correlation and the correlation coefficient increased with the number of associated aDMRs (FIG. 13E). This positive correlation between gene expression and mCG had been observed previously in cultured cell lines as a depletion of methylation in repressive genes located in partially methylated domains (PMDs), resulting in the reactivation of gene expression (Lister et al., Nature 462, 315-322, 2009).

### Example 5

### Shared aDMRs Between the In Vitro Fibroblast/iN Model and Isogenic Entorhinal Cortex Cell Types

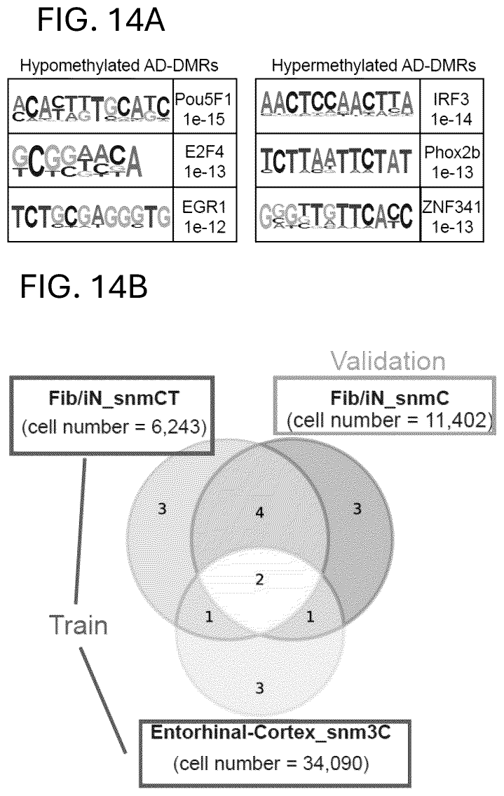

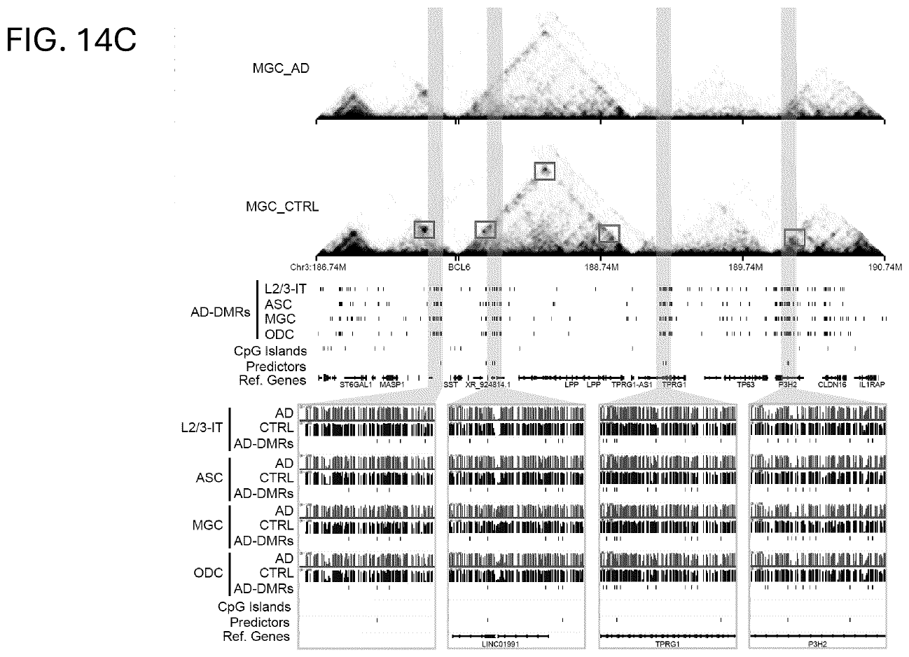

There has been no systematic comparison on genome-wide epigenome between in vitro iN modeling and in vivo primary brain tissues from isogenic patients. Both the entorhinal cortical tissue and cultured fibroblasts and iNs of 2 AD and 1 CTRL donors has been profiled. Overall, there is a small fraction (3,733) of aDMRs overlapping between in vitro fibroblast/iNs and in vivo entorhinal cortex; 2,274 of them have the same direction of methylation changes and are consistent across individuals and in vitro/vivo cell types (FIGS. 7A-7B). To validate the reliability of these aDMRs and investigate individual differences, a random sampling of aDMRs from in vitro or in vivo aDMR pools was done. These were compared to the 2,274 aDMRs between the 2 AD patients and 1 CTRL donor. Distinguishable methylation patterns were evident in the shared aDMRs, whereas those identified from either in vitro or in vivo methods lacked consistency (FIG. 7C). The motif enrichment analysis on these shared DMRs revealed putative TFs candidates (FIG. 14A), like pluripotency associated TF, Pou5F1 (OCT4), as well as early growth response-1 (EGR1) which have already been found to play crucial roles in early stages of AD (Kodamova et al., Neurobiol. Dis. 63, 107-114, 2014; Sun et al., Nat. Commun. 10, 3892, 2019), are specifically enriched in the hypomethylated aDMRs in AD samples.

### Example 6

### A Reliable Set of Differentially Methylated Sites for AD Prediction Identified with a Machine Learning Model

Both cell type variations and individual differences influence the methylation fractions of differentially methylated sites (DMSs) between AD and CTRL groups. To identify DMSs consistent across cell types and resilient to individual variability, a machine learning (ML) method was devised (FIG. 7D). A meticulous iterative feature selection was employed to hone in on candidate CpG sites, resulting in a prediction accuracy of 97.1% to distinguish AD vs., CTRL donors (FIG. 7E). Sites influenced by individual effects were filtered out to enhance the prediction robustness, ultimately pinpointing 859 CpG sites. Although samples exhibited variances between in vivo and in vitro conditions, the methylation patterns of these sites consistently differentiated AD from the CTRL group, remaining stable across diverse cell types and individuals (FIG. 7F-7G). Remarkably, when trained on in vivo snm3C datasets combined with in vitro snmCT datasets and applied to a separate validation dataset (FIG. 14B) using different technologies (snmC-seq, only methylome modality) comprising shared individuals and three unseen donors, the prediction model, grounded on these sites, achieved a flawless accuracy of 100%. Given the robustness of these sites and the predictive model across cell types and donors, they provide valuable insights. The ML-selected CpG sites also have biological importance. For example, 8 ML-selected CpG sites around the BCL6 gene overlapped with aDMRs-enriched TPRG1 and P3H2 genes (FIG. 14C). BCL6 is one of the DEGs in microglia and astrocytes, whereas the TPRG1 locus comprises aDMRs that could work as CREs of the BCL6 gene via loop interactions.

### Discussion

The intricacy of age-related dependencies in human brain neurodegeneration is challenging to recapitulate in cell models, significantly constraining investigation of the molecular mechanisms underlying LOAD pathogenesis. Direct reprogrammed neurons were confirmed to retain aging and AD transcriptomic signatures, providing a novel approach to dissect the biological events occurring in AD brains. For in vitro cellular iN models, six distinct cell states along the iN-induction process were characterized. It was found that AD fibroblast nuclei had a more permissive epigenetic state for ectopic expression of induction factors. The DEGs and DMRs between AD and CTRL in a cell state-specific manner were also identified.

In addition, the first single-cell multi-omics datasets capturing methylome and 3D chromatin conformation on the entorhinal cortex from LOAD patients has been generated in this study. Moreover, brain cell types with isogenic in vitro cultured fibroblasts and iNs derived from the same brain donors were compared. Based on the comparison between the in vitro cellular model and in vivo primary brain tissues, robust aDMR candidates were identified that demonstrated consistency across cell types and individuals. Utilizing ML algorithms using these datasets, a minimum and reliable prediction model to conduct LOAD diagnosis on in vitro cellular fibroblast/iN models and primary brain tissues was developed. Moreover, global chromosomal epigenome erosion in brain cell types from LOAD donors was found, consisting of disrupted active/repressive chromatin compartments, weakened chromatin domain boundaries, and decreased short DNA loop interactions. Furthermore, by integrating this data with published snRNA-seq datasets on LOAD patients, potential CREs interacting with the DEGs involved in the AD process at a cell type-specific level were identified. These findings suggest that an age-dependent dysfunctional genome architecture in brain cell types plays a fundamental role in neurodegeneration. In addition to the current molecular brain cell atlas efforts in the BICCN consortium (Tian et al., bioRxiv 2022.11.30.518285. 10.1101/2022.11.30.518285, 2022), these datasets provide a comprehensive landscape to phenotype the epigenome of the aging brain and AD cognitive disorders.

It will be apparent that the precise details of the methods or compositions described may be varied or modified without departing from the spirit of the described aspects of the disclosure. We claim all such modifications and variations that fall within the scope and spirit of the claims below.

1. A method of identifying a subject as having or at risk of developing Alzheimer's disease (AD), comprising:
obtaining sequence reads of a methylation sequencing assay covering genomic segments of a biological sample from the subject, wherein the genomic segments contain one or more of the genomic positions listed in Table 1 and/or Table 2; and
identifying the subject as having or at risk of developing AD if at least one of the genomic positions has a different methylation status compared to a normal control; or
identifying the subject as not having or at risk of developing AD if none of the genomic positions has a different methylation status compared to a normal control.

2. The method of claim 1, wherein the one or more of the genomic positions listed in Table 1 and/or Table 2 are at least 5, at least 10, at least 15, at least 20, at least 25, at least 30, at least 35, at least 40, at least 45, or at least 50 of the genomic positions listed in Table 1 and/or Table 2, and the method comprises:
identifying the subject as having or at risk of developing AD if all of the genomic positions have a different methylation status compared to a normal control; or
identifying the subject as not having or at risk of developing AD if none of the genomic positions has a different methylation status compared to a normal control.

3. The method of claim 1, wherein the one or more genomic positions are selected from:
chr3:107351515-107351516;
chr1:169668153-169668154;
chr9:114150865-114150866;
chr10:77298787-77298788;
chr1:218669424-218669425;
chr18: 7393790-7393791;
chr16:85241293-85241294;
chr2:78006878-78006879;
chr19: 49468339-49468340;
chr2:171599550-171599551;
chr2: 38079793-38079794;
chr13:29714764-29714765;
chr2:223827178-223827179;
chr13:29697391-29697392;
chr2:223823348-223823349;
chr13:66231576-66231577;
chr4:112701336-112701337;
chr2:54341024-54341025;
chr2: 223389403-223389404; and
chr2: 54323871-54323872.

4. The method of claim 3, wherein the one or more genomic positions consist of:
chr3:107351515-107351516;
chr1:169668153-169668154;
chr9:114150865-114150866;
chr10:77298787-77298788;
chr1:218669424-218669425;
chr18: 7393790-7393791;
chr16:85241293-85241294;
chr2: 78006878-78006879;
chr19: 49468339-49468340;
chr2:171599550-171599551;
chr2: 38079793-38079794;
chr13:29714764-29714765;
chr2:223827178-223827179;
chr13: 29697391-29697392;
chr2: 223823348-223823349;
chr13:66231576-66231577;
chr4:112701336-112701337;
chr2:54341024-54341025;
chr2: 223389403-223389404; and
chr2: 54323871-54323872.

5. The method of claim 1, wherein the biological sample is a single cell or a plurality of cells.

6. The method of claim 5, wherein:
the single cell is a single fibroblast cell;
the single cell is a single induced neuronal (iN) cell;
the plurality of cells is a plurality of fibroblast cells; or
the plurality of cells is a plurality of iN cells.

7. The method of claim 6, wherein the iN cell or iN cells are directly converted from a fibroblast cell or fibroblast cells without going through a stem cell intermediate phase.

8. The method of claim 1, further comprising obtaining the biological sample from the subject.

9. The method of claim 8, wherein the biological sample is obtained by skin biopsy.

10. The method of claim 9, wherein a fibroblast cell or fibroblast cells are obtained from the skin biopsy and are converted into an iN cell or iN cells.

11. The method of claim 1, wherein the genomic segments are up to 300 bases upstream or up to 300 bases downstream of the genomic positions.

12. The method of claim 1, wherein the methylation sequencing assay is a bisulfite sequencing assay.

13. The method of claim 1, further comprising calculating a methylation fraction for each of the genomic positions, wherein the genomic position of the subject has a different methylation status compared to the normal control, if the methylation fraction of the subject is different from the normal control by at least 5%, 10%, 15%, 20%, 25%, 30%, 35%, 40%, 45%, 50%, 55%, 60%, 65%, 70%, 75%, or 80%.

14. The method of claim 1, wherein the AD is late-onset AD.

15. The method of claim 1, further comprising administering a therapeutically effective amount of an AD therapy to the subject if the subject is identified as having or at risk of developing AD.

16. The method of claim 15, wherein the AD therapy comprises administration of a cholinesterase inhibitor, administration of an immunotherapy, administration of an N-methyl-D-aspartate (NMDA) antagonist, or administration of brexpiprazole.

17. A method of identifying a therapeutic agent for the treatment of Alzheimer's disease (AD), comprising:
(i) incubating, in vitro, fibroblast cells or induced neuronal (iN) cells originating from a subject with AD under tissue culture conditions;
(ii) contacting the fibroblast cells or iN cells with a test agent;
(iii) performing a methylation sequencing assay on genomic DNA isolated from the cells following contact with the test agent to identify a methylation status of one or more of the genomic positions listed in Table 1 and/or Table 2; and
(v) identifying the test agent as a therapeutic agent for the treatment of AD if at least one of the genomic positions has a different methylation status compared to control cells not contacted with the test agent; or identifying the test agent as not a therapeutic agent for the treatment of AD if the genomic positions do not have a different methylation status compared to control cells not contacted with the test agent.

18. The method of claim 17, wherein the one or more of the genomic positions listed in Table 1 and/or Table 2 are at least 5, at least 10, at least 15, at least 20, at least 25, at least 30, at least 35, at least 40, at least 45, or at least 50 of the genomic positions listed in Table 1 and/or Table 2.

19. The method of claim 17, wherein the one or more genomic positions are selected from:
chr3:107351515-107351516;
chr1:169668153-169668154;
chr9:114150865-114150866;
chr10:77298787-77298788;
chr1:218669424-218669425;
chr18: 7393790-7393791;
chr16:85241293-85241294;
chr2: 78006878-78006879;
chr19: 49468339-49468340;
chr2:171599550-171599551;
chr2: 38079793-38079794;
chr13:29714764-29714765;
chr2:223827178-223827179;
chr13: 29697391-29697392;
chr2:223823348-223823349;
chr13:66231576-66231577;
chr4:112701336-112701337;
chr2:54341024-54341025;
chr2: 223389403-223389404; and
chr2:54323871-54323872.

20. The method of claim 17, wherein the one or more genomic positions consist of:
chr3:107351515-107351516;
chr1:169668153-169668154;
chr9:114150865-114150866;
chr10:77298787-77298788;
chr1:218669424-218669425;
chr18: 7393790-7393791;
chr16:85241293-85241294;
chr2: 78006878-78006879;
chr19:49468339-49468340;
chr2:171599550-171599551;
chr2: 38079793-38079794;
chr13:29714764-29714765;
chr2:223827178-223827179;
chr13:29697391-29697392;
chr2:223823348-223823349;
chr13:66231576-66231577;
chr4:112701336-112701337;
chr2:54341024-54341025;
chr2: 223389403-223389404; and
chr2: 54323871-54323872.

21. The method of claim 17, wherein the fibroblast cells are obtained from a skin biopsy from a subject with AD.

22. The method of claim 17, wherein the iN cells are directly converted from fibroblast cells obtained from a subject with AD without going through a stem cell intermediate phase.

23. The method of claim 17, wherein the genomic segments are up to 300 bases upstream or up to 300 bases downstream of the genomic positions.

24. The method of claim 17, wherein the AD is late-onset AD.

25. The method of claim 17, further comprising calculating a methylation fraction for each of the genomic positions, wherein the genomic position has a different methylation status compared to the control, if the methylation fraction is different from the control by at least 5%, 10%, 15%, 20%, 25%, 30%, 35%, 40%, 45%, 50%, 55%, 60%, 65%, 70%, 75%, or 80%.
